(19) United States
(12) Patent Application Publication
(10) Pub. No.: US 2006/0015951 A1
(43) Pub. Date: Jan. 19, 2006

(54) Proteins involved in the regulation of energy homeostasis

(76) Inventors: Karsten Eulenberg, Bovenden (DE); Martin Meise, Gottingen (DE); Andreas Molitor, Gottingen (DE); Arnd Steuernagel, Gottingen (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/531,036

(22) PCT Filed: Oct. 14, 2003
(86) PCT No.: PCT/EP03/11352

§ 371 Date: Apr. 12, 2005

(30) Foreign Application Priority Data
Oct. 14, 2002 (EP) 02022880.5
Oct. 22, 2002 (EP) 02023560.2
Nov. 6, 2002 (EP) 02024747.4

(51) Int. Cl. A01K 67/00; A61K 48/00; C07K 14/435; A61K 38/17; C07H 21/04; C12N 5/06

(52) U.S. Cl. 800/8; 514/12; 514/44; 435/69.1; 435/320.1; 435/348; 536/23.5; 530/350

(57) ABSTRACT

The present invention discloses novel uses for energy homeostasis regulating proteins and polynucleotides encoding these in the diagnosis, study, prevention, and treatment of metabolic diseases and disorders.

This invention relates to the use of gut feeling (referred to as guf), GadFly Accession Number CG3811, GadFly Accession Number CG30346 (also referred to as GadFly Accession Number CG8080), retinal degeneration B (referred to as rdgB), Mekk1, Ady43A, EG63B12.4 (referred to as GadFly Accession Number CG14816), twins (referred to as tws), or PP2A-B′ homologous proteins, to the use of polynucleotides encoding these, and to the use of modulators/effectors of the proteins and polynucleotides in the diagnosis, study, prevention, and treatment of obesity and/or diabetes mellitus and/or metabolic syndrome.

There are several metabolic diseases of human and animal metabolism, eg., obesity and severe weight loss, that relate to energy imbalance where caloric intake versus energy expenditure is imbalanced. Obesity is one of the most prevalent metabolic disorders in the world. It is still a poorly understood human disease that becomes as a major health problem more and more relevant for western society. Obesity is defined as a body weight more than 20% in excess of the ideal body weight, frequently resulting in a significant impairment of health. It is associated with an increased risk for cardiovascular disease, hypertension, diabetes, hyperlipidaemia and an increased mortality rate. Besides severe risks of illness, individuals suffering from obesity are often isolated socially.

Obesity is influenced by genetic, metabolic, biochemical, psychological, and behavioral factors, and can be caused by different reasons such as non-insulin dependent diabetes, increase in triglycerides, increase in carbohydrate bound energy and low energy expenditure. As such, it is a complex disorder that must be addressed on several fronts to achieve lasting positive clinical outcome. Since obesity is not to be considered as a single disorder but as a heterogeneous group of conditions with (potential) multiple causes, it is also characterized by elevated fasting plasma insulin and an exaggerated insulin response to oral glucose intake (Koltermann O. G. et al., (1980) J. Clin. Invest 65: 1272-1284). A clear involvement of obesity in type 2 diabetes mellitus can be confirmed (Kopelman P. G., (2000) Nature 404: 635-643).

Hyperlipidemia and elevation of free fatty acids correlate clearly with the metabolic syndrome, which is defined as the linkage between several diseases, including obesity and insulin resistance. This often occurs in the same patients and are major risk factors for development of type 2 diabetes and cardiovascular disease. It was suggested that the control of lipid levels and glucose levels is required to treat type 2 diabetes, heart disease, and other occurances of metabolic syndrome (see, for example, Santomauro A. T. et al., (1999) Diabetes, 48: 1836-1841 and Lakka H. M. et al., (2002) JAMA 288: 2709-2716).

Diabetes is a very disabling disease, because medications do not control blood sugar levels well enough to prevent swinging between high and low blood sugar levels. Patients with diabetes are at risk for major complications, including diabetic ketoacidosis, end-stage renal disease, diabetic retinopathy and amputation. There are also a host of related conditons, such as metabolic syndrome, obesity, hypertension, heart disease, peripheral vascular disease, and infections, for which persons with diabetes are at substantially increased risk. The treatment of these complications contributes to a considerable degree to the enormous cost which is imposed by diabetes on health care systems world wide.

The concept of ‘metabolic syndrome’ (syndrome x, insulin-resistance syndrome, deadly quartet) was first described 1966 by Camus and reintroduced 1988 by Reaven (Camus J. P., (1966) Rev Rhum Mal Osteoartic 33: 10-14; Reaven G. M. et al., (1988) Diabetes, 37: 1595-1607). Today metabolic syndrome is commonly defined as clustering of cardiovascular risk factors like hypertension, abdominal obesity, high blood levels of triglycerides and fasting glucose as well as low blood levels of HDL cholesterol. Insulin resistance greatly increases the risk of developing the metabolic syndrome (Reaven G., (2002) Circulation 106: 286-288). The metabolic syndrome often precedes the development of type II diabetes and cardiovascular disease (Lakka H. M. et al., 2002, supra).

The molecular factors regulating food intake and body weight balance are incompletely understood. Even if several candidate genes have been described which are supposed to influence the homeostatic system(s) that regulate body mass/weight, like leptin or the peroxisome proliferator-activated receptor-gamma co-activator, the distinct molecular mechanisms and/or molecules influencing obesity or body weight/body mass regulations are not known. In addition, several single-gene mutations resulting in obesity have been described in mice, implicating genetic factors in the etiology of obesity (Friedman J. M. and Leibel R. L., (1990) Cell 69: 217-220). In the ob mouse a single gene mutation (obese) results in profound obesity, which is accompanied by diabetes (Friedman J. M. et. al., (1991) Genomics 11: 1054-1062).

Therefore, the technical problem underlying the present invention was to provide for means and methods for modulating (pathological) metabolic conditions influencing body-weight regulation and/or energy homeostatic circuits. The solution to said technical problem is achieved by providing the embodiments characterized in the claims.

Accordingly, the present invention relates to novel functions of proteins and nucleic acids encoding these in body-weight regulation, energy homeostasis, metabolism, and obesity. Further new compositions are provided that are useful in diagnosis, treatment, and prognosis of metabolic diseases and disorders as described.

So far, it has not been described that a protein of the invention or a homologous protein is involved in the regulation of energy homeostasis and body-weight regulation and related disorders, and thus, no functions in metabolic diseases and dysfunctions and other diseases as listed above have been discussed.

Before the present proteins, nucleotide sequences, and methods are described, it is understood that this invention is not limited to the particular methodology, protocols, cell lines, vectors, and reagents described as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies that are reported in the publications which might be used in connection with the invention. Nothing herein is to be construed as an admission that the invention is not entitled to antedate such disclosure.

The present invention discloses that guf (GadFly Accession Number 16747), GadFly Accession Number CG3811, GadFly Accession Number CG30346 (also referred to as GadFly Accession Number CG8080), rdgB (GadFly Accession Number CG11111), Mekk1(GadFly Accession Number CG7717), Ady43A (GadFly Accession Number CG1851), EG63B12.4 (GadFly Accession Number CG14816), tws (GadFly Accession Number CG6235), or PP2A-B′ (GadFly Accession Number CG7913) homologous proteins (herein referred to as “proteins of the invention” or “a protein of the invention”) are regulating the energy homeostasis and fat metabolism, especially the metabolism and storage of triglycerides and glycogen, and polynucleotides, which identify and encode the proteins disclosed in this invention. The invention also relates to vectors, host cells, and recombinant methods for producing the polypeptides and polynucleotides of the invention. The invention also relates to the use of these compounds and effectors/modulators thereof, e.g. antibodies, biologically active nucleic acids, such as antisense molecules, RNAi molecules or ribozymes, aptamers, peptides or low-molecular weight organic compounds recognizing said polynulceotides or polypeptides, in the diagnosis, study, prevention, and treatment of metabolic diseases or dysfunctions, including obesity, diabetes mellitus and/or metabolic syndrome, as well as related disorders such as eating disorder, cachexia, hypertension, coronary heart disease, hypercholesterolemia, dyslipidemia, osteoarthritis, gallstones, or liver fibrosis.

Mammalian polyamines such as spermidine and spermine and their diamine precursor putrescine are known to be involved in the control of the cell cycle and cell growth and are thus essential for mammalian cell growth and viability. The synthesis of polyamines is regulated by a unique feedback mechanism. When cellular concentrations of polyamines increase, expression of ornithine decarboxylase antizyme (ODC-AZ), an ornithine decarboxylase (ODC) inhibitory protein, is induced by polyamine-dependent translational frameshifting. ODC-AZ is the mammalian homolog of the Drosophila gene guf. The antizyme binds to and thereby destabilizes omithine decarboxylase (ODC), which is the first enzyme in polyamine synthesis. After binding of the antizyme, ODC is then rapidly degraded. Antizyme not only inhibits ODC, it also targets the enzyme degradation by the 26S proteasome. Furthermore, it suppresses cellular uptake of polyamines.

Cellular uptake of organic solutes is mediated in large part by a gene family of transporters called organic anion transporting polypeptides (OATPs) (Cal S. Y. et al., (2002) Am J Physiol Gastrointest Liver Physiol 282: G702-710). Organic anion transporting polypeptide 4 (Oatp4; Slc21 a 10; OAT4) is known as a liver-specific and sodium-independent transporter that mediates transport of a variety of compounds. Oatp4 was suggested to be responsible for the homeostasis of endogenous rather than exogenous chemicals, including pharmaceuticals (Li N. et al., (2002) J Pharmacol Exp Ther 301: 551-560). The organic anion transport system is involved in the excretion and reabsorption of various drugs and substances. For example, he multispecific human OAT4 (hOAT4) mediates the high-affinity transport of the nephrotoxic mycotoxin ochratoxin A (Babu E. et al. (2002) Biochim Biophys Acta 1590: 64-75). The uptake of organic anions (such as estrone sulfate) mediated by hOAT4 can be inhibited by the organic anion transport inhibitor probenecid (Enomoto A. et al., (2002) J Pharmacol Exp Ther 301: 797-802). Human OAT4 as well as other anioic transporters mediate the uptake of nonsteroidal anti-inflammatory drugs such as ibuprofen, indomethacin, ketoprofen, or salicylate.

NADP is essential for biosynthetic pathways, energy, and signal transduction. Its synthesis is catalyzed by NAD kinase. The catalytically active homotetramer is highly selective for its substrates, NAD and ATP. ATP-NAD kinase catalyzes the phosphorylation of NAD to NADP utilizing ATP and other nucleoside triphosphates as well as inorganic polyphosphate as a source of phosphorus (Lerner F. et al., (2001) Biochem Biophys Res Commun 288: 69-74). Enzyme activities related to fatty acid synthesis were determined in liver extracts of rats treated with thioacetamide. It was found that the enzymatic activities of ATP-citrate lyase, acetyl CoA carboxylase, fatty acid synthetase, glycerol kinase and NAD-kinase decrease progressively when thioacetamide was chronically administered (Martin-Sanz P. et al. (1989) Carcinogenesis 10:477-481).

The protein tyrosine kinase PYK2 has been implicated in signaling pathways activated by G-protein-coupled receptors, intracellular calcium, and stress signals. PYK2 binding proteins are designated Nirs (PYK2 N-terminal domain-interacting receptors; Nir1, Nir2, and Nir3). Nir proteins bind to the amino-terminal domain of PYK2 via a conserved sequence motif located in the carboxy terminus. The Nir proteins are the human homologues of the Drosophila retinal degeneration B protein (rdgB), a protein implicated in the visual transduction pathway in flies. In addition to the binding of PYK2, Nir proteins are calcium-binding proteins with exhibit phosphoinositol (PI) transfer activity in vivo. Thus, Nir proteins may play a role in the control of calcium and phosphoinositide metabolism downstream of G-protein-coupled receptors (Lev S. et al., (1999) Mol Cell Biol 19: 2278-2288). Human Nir2 is essential for cytokinesis (Litvak V. et al., (2002) Mol Cell Biol 22: 5064-5075). A protein similar to Nir2, the phosphatidylinositol-transfer protein PITP, catalyzes the transfer of phosphatidylinositol (PI) between membranes (Cockcroft. S., (2001) Semin Cell Dev Biol 12:183-191).

A Drosophila mitogen-activated protein kinase kinase (D-MEKK1, MAPK/ERK kinase kinase 1) mediates stress responses through activation of p38 mitogen-activated protein kinase (MAPK). D-MEKK1 kinase activity was activated in animals under conditions of high osmolarity. Drosophila mutants lacking D-MEKK1 were hypersensitive to environmental stresses, including elevated temperature and increased osmolarity. In these D-MEKK1 mutants, activation of Drosophila p38 MAPK in response to stress was poor compared with activation in wild-type animals. D-MEKK1 is structurally similar to the mammalian MAP kinase kinase kinase, MTK1 (also known as MEKK4) (Inoue H. et al. (2001) EMBO J 20: 5421-5430). MTK1 mediates activation of both stress-responsive mitogen-activated protein (MAP) kinases p38 and c-Jun N-terminal kinase (JNK). The MAPK kinase MKK6 is the substrate of MTK1. Growth arrest and DNA damage-inducible 45 (GADD45) proteins were identified as MTK1 activators. GADD45 proteins bind a site in MTK1 near the inhibitory domain and relieve auto-inhibition (Mita H. et al. (2002) Mol Cell Biol 22: 4544-4555). Interleukin-12 and interleukin-18 might activate interferon gamma through GADD45 beta, which activates MEKK4 (Yang J. et al. (2001) Nat Immunol 2:157-164).

Adenosine kinase (ADK) is an abundant enzyme in mammalian tissues that catalyzes the transfer of the gamma-phosphate from ATP to adenosine, thereby serving as a regulator of concentrations of both extracellular adenosine and intracellular adenine nucleotides. Adenosine is a hormone that regulates blood pressure and flow, e.g. in the nephron and other tissues. It causes constriction of blood vessels thereby increasing blood pressure similar to the hormone vasopressin (ADH). Adenosine has widespread effects on the cardiovascular, nervous, respiratory, and immune systems and inhibitors of ADK could play an important pharmacological role in increasing intravascular adenosine concentrations and acting as antiinflammatory agents.

ADK regulates actively the hormone action through phosphorylation. The activity of ADK is significantly impaired in tissues of diabetic rat. It is suggested that insulin increases the rate of ADK gene transcription. Decreased activity of ADK was associated with elevated level of adenosine in diabetic tissues (Sakowicz M. and Pawelczyk T., (2002) Mol Cell Biochem. 236: 163-171). 5′-deoxy-5-iodotubercidin is a potent and selective ADK inhibitor (Lynch J. J, 3rd et al., (1999) Eur J Pharmacol. 364: 141-146.). ADK activity was unchanged in white and brown fat of male diabetic rats or hypothyroidism, but increased with age (Jamal Z. and Saggerson E. D., (1987) Biochem J 245: 881-886). Changes in adenosine concentration may be important in changing insulin sensitivity in adipose tissue whereas changes in adenosine receptor number may be more important in muscle (Newsholme E. A. et al., (1985) FEBS Lett 181: 189-192). However, although a change in activity of ADK by insulin was described in the prior art, it has not been described in the prior art that ADK is directly involved in the regulation of energy homoestasis and thus involved in the storage of triglycerides.

The Drosophila CG 14816 and its homologous human and mouse gene products encode a phosphoglycerate mutase (PGAM) enzyme that catalyzes reactions involving the transfer of phosphogroups between the three carbon atoms of phosphoglycerate. In mammals. PGAM comprises M-, B- and MB-type isozymes composed of the combination of the muscle-specific (M) and nonmuscle-specWic (B) subunits (Uchida K. et al., (1995) Nippon Rinsho 53: 1247-1252). The catalytic mechanism of PGAM involves the formation of a phosphohistidine intermediate (Fothergill-Gilmore L. A. and Watson H. C., (1989) Adv Enzymol Relat Areas Mol Biol 62: 227-313). Phosphoglycerate mutase is an enzyme of the glycolysis and gluconeogenesis that catalyzes the reaction 3-phosphoglycerate into 2-phosphoglycerate and vice versa. The active catalytic core consists of a phosphorylated histidine residue. The enzyme needs a certain amount of 2,3-diphosphoglycerate as a primer for the reaction thereby transfering the 2- or 3-phosphoryl group on the histidine in the active centre.

The Drosophila tws gene product is a regulatory subunit of protein phosphatase 2 (PP2A), one of the major classes of serine/threonine phosphatases (Uemura T. et al., (1993) Genes Dev. 7: 429-440). In the fruit fly, tws is involved in the control of cell division (Omel'ianchuk L. V. et al., (1997) Genetika. 33: 1494-1501). The beta-catalytic subunit of protein phosphatase 2A (PP2A) is a member of the bestrophin complex. Bestrophin is in the signal transduction pathway that it is regulated by phosphorylation, and that phosphorylation of bestrophin is regulated by PP2A (Marmorstein L. Y. et al., (2002) J Biol Chem 277: 30591-30597). Type 2A protein phosphatase (PP2A) comprises a diverse family of phosphoserine- and phosphothreonine-specific enzymes ubiquitously expressed in eukaryotic cells. Common to all forms of PP2A is a catalytic subunit (PP2Ac) which can form two distinct complexes, one with a structural subunit termed PR65/A and another with an alpha4 protein. The PR65/A-PP2Ac dimer may further associate with a regulatory subunit and form a trimeric holoenzyme. To date, three distinct families of regulatory subunits, which control substrate selectivity and phosphatase activity and target PP2A holoenzymes to their substrates, have been identified. Other molecular mechanisms that regulate PP2Ac function include phosphorylation, carboxyl methylation, inhibition by intracellular protein inhibitors (I(1)(PP2A) and I(2) (PP2A)), and stimulation by ceramide. PP2A dephosphorylates many proteins in vitro, but in vivo protein kinases and transcription factors appear to represent two major sets of substrates. (Zolnierowicz S., (2000) Biochem Pharmacol. 60: 1225-1235). Protein phosphatase 2A (PP2A) is a multimeric enzyme, containing a catalytic subunit complexed with two regulatory subunits. PP2A exerts a range of cellular functions including cell cycle regulation and cell fate determination. (Gotz J. et al., (1998) Proc Natl Acad Sci USA 95: 12370-12375).

PP2A has been implicated in a variety of regulatory processes including cell growth and division, muscle contraction, and gene transcription (Janssens V. and Goris J., (2001) Biochem J. 353: 417-439). PP2A is composed of a 36-kD catalytic subunit C (PP2AC), a highly homologous 65-kD structural subunit (PR65), and any of several different regulatory subunits (B, B′, B″B′″) that control its specificity. The B56 family (PR61; B′) of regulatory subunits is encoded by at least 5 homologous but distinct genes, termed B56-alpha, -beta, -gamma, -delta, and epsilon (McCright B. et al., (1996) J Biol Chem 271: 22081-22089). At least three different splice variants exist of the human regulatory subunits B56-gamma (gamma1, gamma2, gamma3). B56-gamma1, -gamma2, -gamma3 are localized in the nucleus, B56-delta is found in both the nucleus and the cytoplasm. B56-gamma, gamma2, -gamma3 are widely expressed (abundant in heart and muscle), B56-delta is predominantly expressed in brain (McCright B. and Virshup D. M., (1995) J Biol Chem. 270: 26123-26128; Tehrani M. A. et al., (1996) J Biol Chem. 271: 5164-5170). PP2AC expression level changes during adipocyte differentiation induced by PPARgamma (Altiok S. et al., (1997) Genes Dev. 11: 1987-1998). PP2AC and all B′(PR61; B56) isoforms interact with APC (scaffolding protein for assembly of beta-catenin axin and GSK-3beta). B56 may direct PP2A to dephosphorylate specific components of the APC-dependent signaling complex and thereby inhibit Wnt signaling (Seeling J. M. et al., (1999) Science. 283: 2089-2091).

guf, CG381 1, CG30346 (CG8080), rdgB, Mekk1, Ady43A, CG14816, tws, or PP2A-B′ homologous proteins and nucleic acid molecules coding therefore are obtainable from insect or vertebrate species, e.g. mammals or birds. Particularly preferred are homologous nucleic acids, particularly nucleic acids encoding a human homologous protein as described in Table 1.

The invention particularly relates to a nucleic acid molecule encoding a polypeptide contributing to regulating the energy homeostasis and the metabolism of triglycerides and/or glycogen, wherein said nucleic acid molecule comprises
(a) the nucleotide sequence of Drosophila guf, CG3811, CG30346 (CG8080), rdgB, Mekk1, Ady43A, CG14816, tws, or PP2A-B′ homologous nucleic acids, particularly nucleic acids as described in Table 1, and/or a sequence complementary thereto,
(b) a nucleotide sequence which hybridizes at 50° C. in a solution containing 1×SSC and 0.1% SDS to a sequence of (a),
(c) a sequence corresponding to the sequences of (a) or (b) within the degeneration of the genetic code,
(d) a sequence which encodes a polypeptide which is at least 85%, preferably at least 90%, more preferably at least 95%, more preferably at least 98% and up to 99.6% identical to the amino acid sequences of the guf, CG3811, CG30346 (CG8080), rdgB, Mekk1, Ady43A, CG14816, tws, or PP2A-B′ homologous protein, particularly to a human homologous protein as described in Table 1,
(e) a sequence which differs from the nucleic acid molecule of (a) to (d) by mutation and wherein said mutation causes an alteration, deletion, duplication and/or premature stop in the encoded polypeptide or
(f) a partial sequence of any of the nucleotide sequences of (a) to (e) having a length of 15-25 bases, preferably 25-35 bases, more preferably 35-50 bases, and most preferably at least 50 bases.

The invention is based on the finding that guf, CG3811, CG30346 (CG8080), rdgB, Mekk1, Ady43A, CG14816, tws, or PP2A-B′ and the polynucleotides encoding these, are involved in the regulation of triglyceride and/or glycogen storage and therefore energy homeostasis. The invention describes the use of these compositions comprising the polynucleotides, polypeptides and modulators/effectors thereof for the diagnosis, study, prevention, or treatment of metabolic diseases or dysfunctions, including obesity, diabetes mellitus and/or metabolic syndrome, as well as related disorders such as eating disorder, cachexia, hypertension, coronary heart disease, hypercholesterolemia, dyslipidemia, osteoarthritis, gallstones or liver fibrosis.

Accordingly, the present invention relates to genes with novel functions in body-weight regulation, energy homeostasis, metabolism, and obesity, functional fragments of said genes, polypeptides encoded by said genes or functional fragments thereof, and modulators/effectors thereof, e.g. antibodies, biologically active nucleic acids, such as antisense molecules, RNAi molecules, or ribozymes, aptamers, peptides or low-molecular weight organic compounds recognizing said polynucleotides or polypeptides.

The ability to manipulate and screen the genomes of model organisms such as the fly Drosophila melanogaster provides a powerful tool to analyze biological and biochemical processes that have direct relevance to more complex vertebrate organisms due to significant evolutionary conservation of genes, cellular processes, and pathways (see, for example, Adams M. D. et al., (2000) Science 287: 2185-2195). Identification of novel gene functions in model organisms can directly contribute to the elucidation of correlative pathways in mammals (humans) and of methods of modulating them. A correlation between a pathology model (such as changes in triglyceride levels as indication for metabolic syndrome including obesity) and the modified expression of a fly gene can identify the association of the human ortholog with the particular human disease.

A forward genetic screen was performed in fly displaying a mutant phenotype due to misexpression of a known gene (see, St Johnston D., (2002) Nat Rev Genet 3: 176-188; Rorth P., (1996) Proc Natl Acad Sci USA 93: 12418-12422). In this invention, we have used a genetic screen to identify mutations that cause changes in the body weight, which are reflected by a significant change of triglyceride and/or glycogen levels.

Obese people mainly show a significant increase in the content of triglycerides. Triglycerides are the most efficient storage for energy in cells. In order to isolate genes with a function in energy homeostasis, several thousand EP-lines were tested for their triglyceride and/or glycogen content after a prolonged feeding period (see Examples for more detail). Lines with significantly changed triglyceride and/or glycogen content were selected as positive candidates for further analysis. The change of triglyceride and/or glycogen content due to the loss of a gene function suggests gene activities in energy homeostasis in a dose dependent manner that controls the amount of energy stored as triglycerides and/or glycogen.

In this invention, the content of triglycerides of a pool of flies with the same genotype after feeding for six days was analyzed using a triglyceride and glycogen assay. Male flies homozygous, hemizygous, or heterozygous for the integration of vectors in Drosophila lines were analyzed in an assay measuring the triglyceride contents of these flies and in an assay measuring the glycogen contents of these flies, illustrated in more detail in the Examples section. The results of the triglyceride and/or glycogen content analysis are shown in FIGS. 1, 4, 7, 10, 14, 17, 20, 23, and 25, respectively.

An additional screen using Drosophila mutants with modifications of the eye phenotype identified a modification of UCP activity by PP2A-B′, thereby leading to an altered mitochondrial activity.

These findings suggest the presence of similar activities of these described homologous proteins in humans that provides insight into diagnosis, treatment, and prognosis of metabolic disorders. Genomic DNA sequences were isolated that are localized adjacent to the EP vector integration. Using those isolated genomic sequences public databases like Berkeley Drosophila Genome Project (GadFly; see also FlyBase (1999) Nucleic Acids Research 27: 85-88) were screened thereby identifying the integration sites of the vectors, and the corresponding genes, described in more detail in the Examples section. The molecular organization of the gene is shown in FIGS. 2, 5, 8,11, 15, 18, 21, 24, and 26, respectively.

The Drosophila guf, CG3811, CG30346 (CG8080), rdgB, Mekk1, Ady43A, CG14816, tws, and PP24-B′ genes and proteins encoded thereby with functions in the regulation of triglyceride and/or glycogen metabolism were further analysed in publicly available sequence databases (see Examples for more detail) and mammalian homologs were identified.

The function of the mammalian homologs in energy homeostasis was further validated in this invention by analyzing the expression of the transcripts in different tissues and by analyzing the role in adipocyte differentiation. Expression profiling studies (see Examples for more detail) confirm the particular relevance of the proteins of the invention regulators of energy metabolism in mammals. Further, we show that the proteins of the invention are regulated by fasting and by genetically induced obesity. In this invention, we used mouse models of insulin resistance and/or diabetes, such as mice carrying gene knockouts in the leptin pathway (for example, ob (leptin) or db (leptin receptor) mice) to study the expression of the proteins of the invention. Such mice develop typical symptoms of diabetes, show hepatic lipid accumulation and frequently have increased plasma lipid levels (see Bruning J. C. et al., (1998) Mol. Cell. 2:559-569).

Microarrays are analytical tools routinely used in bioanalysis. A microarray has molecules distributed over, and stably associated with, the surface of a solid support. The term “microarray” refers to an arrangement of a plurality of polynucleotides, polypeptides, antibodies, or other chemical compounds on a substrate. Microarrays of polypeptides, polynucleotides, and/or antibodies have been developed and find use in a variety of applications, such as monitoring gene expression, drug discovery, gene sequencing, gene mapping, bacterial identification, and combinatorial chemistry. One area in particular in which microarrays find use is in gene expression analysis (see Example 6). Array technology can be used to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for identifying genes that are tissue specific, are affected by a substance being tested in a toxicology assay, are part of a signaling cascade, carry out housekeeping functions, or are specifically related to a particular genetic predisposition, condition, disease, or disorder.

Microarrays may be prepared, used, and analyzed using methods known in the art (see for example, Brennan T. M., (1995) U.S. Pat. No. 5,474,796; Schena M. et al., (1996) Proc. Natl. Acad. Sci. USA 93: 10614-10619; Baldeschwieler et al., (1995) PCT application WO9525116; Shalon T. D. and Brown P. O., (1995) PCT application WO9535505; Heller R. A. et al., (1997) Proc. Natl. Acad. Sci. USA 94: 2150-2155; Heller M. J. and Tu E., (1997) U.S. Pat. No. 5,605,662). Various types of microarrays are well known and thoroughly described in Schena M., ed. (1999); DNA Microarrays: A Practical Approach, Oxford University Press, London.

Oligonucleotides or longer fragments derived from any of the polynucleotides described herein may be used as elements on a microarray. The microarray can be used in transcript imaging techniques, which monitor the relative expression levels of large numbers of genes simultaneously as described below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patent. For example, therapeutic agents, which are highly effective and display the fewest side effects may be selected for a patient based on his/her pharnacogenomic profile.

As determined by microarray analysis, ornithine decarboxylase antizyme 1 (OAZ1) and ornithine decarboxylase antizyme 2 (OAZ2) are strong candidates for the manufacture of a pharmaceutical composition and a medicament for the treatment of conditions related to human metabolism, such as obesity, diabetes, and/or metabolic syndrome.

The invention also encompasses polynucleotides that encode the proteins of the invention or homologous proteins. Accordingly, any nucleic acid sequence, which encodes an amino acid sequence of the proteins of the invention or homologous proteins, can be used to generate recombinant molecules that express the proteins of the invention or homologous proteins. In a particular embodiment, the invention encompasses a nucleic acid encoding Drosophila guf, CG3811, CG30346 (CG8080), rdgB, Mekk1, Ady43A, CG14816, tws, or PP2A-B′, or human guf, CG3811, CG30346 (CG8080), rdgB, Mekk1, Ady43A, CG14816, tws, or PP2A-B′ homologs, preferably human homologous proteins as described in Table 1; referred to herein as the proteins of the invention. It will be appreciated by those skilled in the art that as a result of the degeneracy of the genetic code, a multitude of nucleotide sequences encoding the proteins, some bearing minimal homology to the nucleotide sequences of any known and naturally occurring gene, may be produced. The invention contemplates each and every possible variation of nucleotide sequence that can be made by selecting combinations based on possible codon choices.

Also encompassed by the invention are polynucleotide sequences that are capable of hybridizing to the claimed nucleotide sequences, and in particular, those of the polynucleotides encoding the proteins of the invention, under various conditions of stringency. Hybridization conditions are based on the melting temperature (Tm) of the nucleic acid binding complex or probe, as taught in Wahl, G. M. et al. (1987: Methods Enzymol. 152: 399-407) and Kimmel, A. R. (1987; Methods Enzymol. 152: 507-511), and may be used at a defined stringency. Preferably, hybridization under stringent conditions means that after washing for 1 h with 1×SSC and 0.1% SDS at 50° C., preferably at 55° C., more preferably at 62° C. and most preferably at 68° C., particularly for 1 h in 0.2×SSC and 0.1% SDS at 50° C., preferably at 55° C., more preferably at 62° C. and most preferably at 68° C., a positive hybridization signal is observed. Altered nucleic acid sequences encoding the proteins which are encompassed by the invention include deletions, insertions or substitutions of different nucleotides resulting in a polynucleotide that encodes the same or a functionally equivalent protein.

The encoded proteins may also contain deletions, insertions or substitutions of amino acid residues, which produce a silent change and result in functionally equivalent proteins. Deliberate amino acid substitutions may be made on the basis of similaity in polarity, charge, solubility, hydrophobicity, hydrophilicity, and/or the amphipathic nature of the residues as long as the biological activity of the protein is retained. Furthermore, the invention relates to peptide fragments of the proteins or derivatives thereof such as cyclic peptides, retro-inverso peptides of peptide mimetics having a length of at least 4, preferably at least 6 and up to 50 amino acids.

Also included within the scope of the present invention are alleles of the genes encoding the proteins of the invention and homologous proteins. As used herein, an ‘allele’ or ‘allelic sequence’ is an alternative form of the gene, which may result from at least one mutation in the nucleic acid sequence. Alleles may result in altered mRNAs or polypeptides whose structures or function may or may not be altered. Any given gene may have none, one or many allelic forms. Common mutational changes, which give rise to alleles, are generally ascribed to natural deletions, additions or substitutions of nucleotides. Each of these types of changes may occur alone or in combination with the others, one or more times in a given sequence.

The nucleic acid sequences encoding the proteins of the invention or homologous proteins may be extended utilizing a partial nucleotide sequence and employing various methods known in the art to detect upstream sequences such as promoters and regulatory elements.

In order to express a biologically active protein, the nucleotide sequences encoding the proteins or functional equivalents, may be inserted into appropriate expression vectors, i.e., a vector which contains the necessary elements for the transcription and translation of the inserted coding sequence. Methods, which are well known to those skilled in the art, may be used to construct expression vectors containing sequences encoding the proteins and the appropriate transcriptional and translational control elements. Regulatory elements include for example an promoter, an initiation codons, a stop codon, a mRNA stability regulatory element, and a polyadenylation signal. Expression of a polynucleotide can be assured by (i) consetutive promoters such as the Cytomegalovirus (CMV) promoter/enhancer region, (ii) tissue specific promoters such as the insulin promoter (see, Soria B. et al., (2000) Diabetes 49: 157-162), SOX2 gene promoter (see Li M. et al., (1998) Curr. Biol. 8: 971-974), Msi-1 promoter (see Sakakibara S. and Okano H., (1997) J. Neuroscience 17: 8300-8312), alpha-cardia myosin heavy chain promoter or human atrial natriuretic factor promoter (Klug M. G.et al., (1996) J. Clin. Invest. 98: 216-224; Wu J. et al., (1989) J. Biol. Chem. 264: 6472-6479)or (iii) inducible promoters such as the tetracycline inducible system. Expression vectors can also contain a selection agent or marker gene that confers antibiotic resistance such as the neomycin, hygromycin or puromycin resistance genes. These methods include in vitro recombinant DNA techniques, synthetic techniques, and in vivo genetic recombination. Such techniques are described in Sambrook J. et al. (1989) Molecular Cloning, A Laboratory Manual, Cold Spring Harbor Press, Plainview, N.Y. and Ausubel F. M. et al. (1989) Current Protocols in Molecular Biology, John Wiley & Sons, New York, N.Y.

In a further embodiment of the invention, natural, modified or recombinant nucleic acid sequences encoding the proteins of the invention or homologous proteins may be ligated to a heterologous sequence to encode a fusion protein.

A variety of expression vector/host systems may be utilized to contain and express sequences encoding the proteins or fusion proteins. These include, but are not limited to, micro-organisms such as bacteria transformed with recombinant bacteriophage, plasmid or cosmid DNA expression vectors; yeast transformed with yeast expression vectors; insect cell systems infected with virus expression vectors (e.g., baculovirus, adenovirus, adeno-associated virus, lentiverus, retrovirus); plant cell systems transformed with virus expression vectors (e.g., cauliflower mosaic virus, CaMV; tobacco mosaic virus, TMV) or with bacterial expression vectors (e.g., Ti or PBR322 plasmids); or animal cell systems.

The presence of polynucleotide sequences of the invention in a sample can be detected by DNA-DNA or DNA-RNA hybridization or amplification using probes or portions or fragments of said polynucleotides. Nucleic acid amplification based assays involve the use of oligonucleotides or oligomers based on the sequences specific for the gene to detect transformants containing DNA or RNA encoding the corresponding protein. As used herein ‘oligonucleotides’ or ‘oligomers’ refer to a nucleic acid sequence of at least about 10 nucleotides and as many as about 60 nucleotides, preferably about 15 to 30 nucleotides, and more preferably about 20-25 nucleotides, which can be used as a probe or amplimer.

A wide variety of labels and conjugation techniques are known by those skilled in the art and may be used in various nucleic acid and amino acid assays. Means for producing labeled hybridization or PCR probes for detecting polynucleotide sequences include oligo-labeling, nick translation, end-labeling of RNA probes, PCR amplification using a labeled nucleotide, or enzymatic synthesis. These procedures may be conducted using a variety of commercially available kits (Pharmacia & Upjohn, (Kalamazoo, Mich.); Promega (Madison Wis.); and U.S. Biochemical Corp., (Cleveland, Ohio).

The presence of proteins of the invention in a sample can be determined by immunological methods or activity measurement. A variety of protocols for detecting and measuring the expression of proteins, using either polyclonal or monoclonal antibodies specific for the protein or reagents for determining protein activity are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on the protein is preferred, but a competitive binding assay may be employed. These and other assays are described, among other places, in Hampton R. et al. (1990; Serological Methods, a Laboratory Manual, APS Press, St Paul, Minn.) and Maddox D. E. et al. (1983; J. Exp. Med. 158:1211-1226).

Suitable reporter molecules or labels, which may be used, include radionuclides, enzymes, fluorescent, chemiluminescent or chromogenic agents as well as substrates, co-factors, inhibitors, magnetic particles, and the like. Host cells transformed with nucleotide sequences encoding a protein of the invention may be cultured under conditions suitable for the expression and recovery of said protein from cell culture. The protein produced by a recombinant cell may be secreted or contained intracellularly depending on the sequence or/and the vector used. As will be understood by those of skill in the art, expression vectors containing polynucleotides, which encode the protein may be designed to contain signal sequences, which direct secretion of the protein through a prokaryotic or eukaryotic cell membrane. Other recombinant constructions may be used to join sequences encoding the protein to nucleotide sequence encoding a polypeptide domain, which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAG extension/affinity purification system (Immunex Corp., Seattle, Wash.) The inclusion of cleavable linker sequences such as those specific for Factor XA or Enterokinase (Invitrogen, San Diego, Calif.) between the purification domain and the desired protein may be used to facilitate purification.

Diagnostics and Therapeutics

The data disclosed in this invention show that the nucleic acids and proteins of the invention and effectors/modulators thereof are useful in diagnostic and therapeutic applications implicated, for example but not limited to, in metabolic diseases or dysfunctions, including obesity, diabetes and/or metabolic syndrome, as well as related disorders such as eating disorder, cachexia, hypertension, coronary heart disease, hypercholesterolemia, dyslipidemia, osteoarthritis, gallstones, or liver fibrosis. Hence, diagnostic and therapeutic uses for the proteins of the invention nucleic acids and proteins of the invention are, for example but not limited to, the following: (i) protein therapy, (ii) small molecule drug target, (iii) antibody target (therapeutic, diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues).

The nucleic acids and proteins of the invention and modularors/effectors thereof are useful in diagnostic and therapeutic applications implicated in various applications as described below. For example, but not limited to, cDNAs encoding the proteins of the invention and particularly their human homologues may be useful in gene therapy, and the proteins of the invention and particularly their human homologues may be useful when administered to a subject in need thereof. By way of non-limiting example, the compositions of the present invention will have efficacy for treatment of patients suffering from, for example, but not limited to, in metabolic disorders as described above.

The nucleic acids of the invention or fragments thereof, may further be useful in diagnostic applications, wherein the presence or amount of the nucleic acids or the proteins are to be assessed. Further antibodies that bind immunospecifically to the novel substances of the invention may be used in therapeutic or diagnostic methods.

For example, in one aspect, antibodies, which are specific for a protein of the invention or a homologous protein, may be used directly as a modulator/effector, e.g. an antagonist or an antagonist or indirectly as a targeting or delivery mechanism for bringing a pharmaceutical agent to cells or tissue which express the protein. The antibodies may be generated using methods that are well known in the art. Such antibodies may include, but are not limited to, polyclonal, monoclonal, chimeric single chain, Fab fragments, and fragments produced by a Fab expression library. Neutralising antibodies, (i.e., those which inhibit dimer formation) are especially preferred for therapeutic use.

For the production of antibodies, various hosts including goats, rabbits, rats, mice, humans, and others, may be immunized by injection with the protein or any fragment or oligopeptide thereof which has immunogenic properties. Depending on the host species, various adjuvants may be used to increase immunological response. It is preferred that the peptides, fragments or oligopeptides used to induce antibodies to the protein have an amino acid sequence consisting of at least five amino acids, and more preferably at least 10 amino acids.

Monoclonal antibodies to the proteins may be prepared using any technique that provides for the production of antibody molecules by continuous cell lines in culture. These include, but are not limited to, the hybridoma technique, the human B-cell hybridoma technique, and the EBV-hybridoma technique (Kohler G. et al., (1975) Nature 256: 495-497; Kozbor D. et al., (1985) J. Immunol. Methods 81: 31-42; Cote R. J. et al., (1983) Proc. Nati. Acad. Sci. 80: 2026-2030; Cole S. P. et al., (1984) Mol. Cell Biol. 62:109-120).

In addition, techniques developed for the production of ‘chimeric antibodies’, the splicing of mouse antibody genes to human antibody genes to obtain a molecule with appropriate antigen specificity and biological activity can be used (Morrison S. L. et al., (1984) Proc. Natl. Acad. Sci. 81: 6851-6855; Neuberger M. S. et al., (1984) Nature 312: 604-608; Takeda S. et al., (1985) Nature 314: 452-454). Alternatively, techniques described for the production of single chain antibodies may be adapted, using methods known in the art, to produce single chain antibodies specific for the proteins of the invention and homologous proteins. Antibodies with related specificity, but of distinct idiotypic composition, may be generated by chain shuffling from random combinatorial immunoglobulin libraries (Kang A. S. et al., (1991) Proc. Natl. Acad. Sci. 88: 11120-11123). Antibodies may also be produced by inducing in vivo production in the lymphocyte population or by screening recombinant immunoglobulin libraries or panels of highly specific binding reagents as disclosed in the literature (Orlandi R. et al., (1989) Proc. Natl. Acad. Sci. 86: 3833-3837; Winter G. and Milstein C., (1991) Nature 349: 293-299).

Antibody fragments which contain specific binding sites for the proteins may also be generated. For example, such fragments include, but are not limited to, the F(ab′)₂fragments which can be produced by Pepsin digestion of the antibody molecule and the Fab fragments which can be generated by reducing the disulfide bridges of F(ab′)₂ fragments. Alternatively, Fab expression libraries may be constructed to allow rapid and easy identification of monoclonal Fab fragments with the desired specificity (Huse W. D. et al., (1989) Science 246: 1275-1281).

Various immunoassays may be used for screening to identify antibodies having the desired specificity. Numerous protocols for competitive binding and immunoradiometric assays using either polyclonal or monoclonal antibodies with established specificities are well known in the art. Such immunoassays typically involve the measurement of complex formation between the protein and its specific antibody. A two-site, monoclonal-based immunoassay utilizing monoclonal antibodies reacive to two non-interfering protein epitopes are preferred, but a competitive binding assay may also be employed (Maddox, supra).

In another embodiment of the invention, the polynucleotides of the invention or fragments thereof or nucleic acid modulator/effector molecules such as aptamers, antisense molecules, RNAi molecules, or ribozymes may be used for therapeutic purposes. In one aspect, aptamers, i.e. nucleic acid molecules, which are capable of binding to a protein of the invention and modulating its activity, may be generated by a screening and selection procedure involving the use of combinatorial nucleic acid libraries.

In a further aspect, antisense molecules may be used in situations in which it would be desirable to block the transcription of the mRNA. In particular, cells may be transformed with sequences complementary to polynucleotides encoding the proteins of the invention or homologous proteins. Thus, antisense molecules may be used to modulate protein activity or to achieve regulation of gene function. Such technology is now well known in the art, and sense or antisense oligomers or larger fragments, can be designed from various locations along the coding or control regions of sequences encoding the proteins. Expression vectors derived from retroviruses, adenovirus, herpes or vaccinia viruses or from various bacterial plasmids may be used for delivery of nucleotide sequences to the targeted organ, tissue or cell population. Methods, which are well known to those skilled in the art, can be used to construct recombinant vectors, which will express antisense molecules complementary to the polynucleotides of the genes encoding the proteins of the invention and homologous proteins. These techniques are described both in Sambrook et al. (supra) and in Ausubel et al. (supra). Genes encoding the proteins of the invention or homologous proteins can be turned off by transforming a cell or tissue with expression vectors, which express high levels of polynucleotides that encode the proteins of the invention or homologous proteins or functional fragments thereof. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector and even longer if appropriate replication elements are part of the vector system.

As mentioned above, modifications of gene expression can be obtained by designing antisense molecules, e.g. DNA, RNA or PNA, to the control regions of the genes encoding the proteins of the invention and homologous proteins, i.e., the promoters, enhancers, and introns. Oligonucleotides derived from the transcription initiation site, e.g., between positions −10 and +10 from the start site, are preferred. Similarly, inhibition can be achieved using “triple helix” base-pairing methodology. Triple helix pairing is useful because it cause inhibition of the ability of the double helix to open sufficiently for the binding of polymerases, transcription factors or regulatory molecules. Recent therapeutic advances using triplex DNA have been described in the literature (Gee J. E. et al., (1994) Gene 149: 109-114; Huber B. E. and Carr B. I., Molecular and Immunologic Approaches, Futura Publishing Co., Mt. Kisco, N.Y.). The antisense molecules may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. Examples, which may be used, include engineered hammerhead motif ribozyme molecules that can be specifically and efficiently catalyze endonucleolytic cleavage of sequences encoding the proteins of the invention and homologous proteins. Specific ribozyme cleavage sites within any potential RNA target are initially identified by scanning the target molecule for ribozyme cleavage sites which include the following sequences: GUA, GUU, and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Antisense molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences. Such DNA sequences may be incorporated into a variety of vectors with suitable RNA polymerase promoters such as T7 or SP6. Alternatively, these cDNA constructs that synthesize antisense RNA constitutively or inducibly can be introduced into cell lines, cells or tissues. RNA molecules may be modified to increase intracellular stability and half-life. Possible modifications include, but are not limited to, the addition of flanking sequences at the 5′ and/or 3′ ends of the molecule or modifications in the nucleobase, sugar and/or phosphate moieties, e.g. the use of phosphorothioate or 2′ O-methyl rather than phosphodiesterase linkages within the backbone of the molecule. This concept is inherent in the production of PNAs and can be extended in all of these molecules by the inclusion of non-traditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection and by liposome injections may be achieved using methods, which are well known in the art. Any of the therapeutic methods described above may be applied to any suitable subject including, for example, mammals such as dogs, cats, cows, horses, rabbits, monkeys, and most preferably, humans.

An additional embodiment of the invention relates to the administration of a pharmaceutical composition, in conjunction with a pharmaceutically acceptable carrier, for any of the therapeutic effects discussed above. Such pharmaceutical compositions may consist of the nucleic acids and the proteins of the invention and homologous nucleic acids or proteins, antibodies to the proteins of the invention and homologous proteins, mimetics, agonists, antagonists or inhibitors of the proteins of the invention and homologous proteins or nucleic acids. The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a patient alone or in combination with other agents, drugs or hormones. The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual or rectal means.

In addition to the active ingredients, these pharmaceutical compositions may contain suitable pharmaceutically-acceptable carriers comprising excipients and auxiliaries, which facilitate processing of the active compounds into preparations, which can be used pharmaceutically. Further details on techniques for formulation and administration may be found in the latest edition of Remington's Pharmaceutical Sciences (Maack Publishing Co., Easton, Pa.).

Pharmaceutical compositions suitable for use in the invention include compositions wherein the active ingredients are contained in an effective amount to achieve the intended purpose. The determination of an effective dose is well within the capability of those skilled in the art. For any compounds, the therapeutically effective dose can be estimated initially either in cell culture assays, e.g., of preadipocyte cell lines or in animal models, usually mice, rabbits, dogs or pigs. The animal model may also be used to determine the appropriate concentration range and route of administration. Such information can then be used to determine useful doses and routes for administration in humans. A therapeutically effective dose refers to that amount of active ingredient, for example a nucleic acid or a protein of the invention or an antibody, which is sufficient for treating a specific condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. Pharmaceutical compositions, which exhibit large therapeutic indices, are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage from employed, sensitivity of the patient, and the route of administration. The exact dosage will be determined by the practitioner, in light of factors related to the subject that requires treatment. Dosage and administration are adjusted to provide sufficient levels of the active moiety or to maintain the desired effect. Factors, which may be taken into account, include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week or once every two weeks depending on half-life and clearance rate of the particular formulation. Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art employ different formulations for nucleotides than for proteins or their inhibitors. Similarly, delivery of polynucleotides or polypeptides will be specific to particular cells, conditions, locations, etc.

In another embodiment, antibodies which specifically bind to the proteins may be used for the diagnosis of conditions or diseases characterized by or associated with over- or underexpression of the proteins of the invention and homologous proteins or in assays to monitor patients being treated with the proteins of the invention and homologous proteins, or effectors thereof, e.g. agonists, antagonists, or inhibitors. Diagnostic assays include methods which utilize the antibody and a label to detect the protein in human body fluids or extracts of cells or tissues. The antibodies may be used with or without modification, and may be labeled by joining them, either covalently or non-covalently, with a reporter molecule. A wide variety of reporter molecules which are known in the art may be used several of which are described above.

A variety of protocols including ELISA, RIA, and FACS for measuring proteins are known in the art and provide a basis for diagnosing altered or abnormal levels of gene expression. Normal or standard values for gene expression are established by combining body fluids or cell extracts taken from normal mammalian subjects, preferably human, with antibodies to the protein under conditions suitable for complex formation. The amount of standard complex formation may be quantified by various methods, but preferably by photometry, means. Quantites of protein expressed in control and disease, samples from biopsied tissues are compared with the standard values. Deviation between standard and subject values establishes the parameters for diagnosing disease.

In another embodiment of the invention, the polynucleotides specific for the proteins of the invention and homologous proteins may be used for diagnostic purposes. The polynucleotides, which may be used, include oligonucleotide sequences, antisense RNA and DNA molecules, and PNAs. The polynucleotides may be used to detect and quantitate gene expression in biopsied tissues in which gene expression may be correlated with disease. The diagnostic assay may be used to distinguish between absence, presence, and excess gene expression, and to monitor regulation of protein levels during therapeutic intervention.

In one aspect, hybridization with probes which are capable of detecting polynucleotide sequences, including genomic sequences, encoding the proteins of the invention and homologous proteins or closely related molecules, may be used to identify nucleic acid sequences which encode the respective protein. The hybridization probes of the subject invention may be DNA or RNA and derived from the nucleotide sequence of the polynucleotide encoding the proteins of the invention or from a genomic sequence including promoter, enhancer elements, and introns of the naturally occurring gene. Hybridization probes may be labeled by a variety of reporter groups, for example, radionuclides such as ³²P or ³⁵S or enzymatic labels, such as alkaline phosphatase coupled to the probe via avidin/biotin coupling systems, and the like.

Polynucleotide sequences specific for the proteins of the invention and homologous nucleic acids may be used for the diagnosis of conditions or diseases, which are associated with the expression of the proteins. Examples of such conditions or diseases include, but are not limited to, pancreatic diseases and disorders, including diabetes. Polynucleotide sequences specific for the proteins of the invention and homologous proteins may also be used to monitor the progress of patients receiving treatment for pancreatic diseases and disorders, including diabetes. The polynucleotide sequences may be used qualitative or quantitative assays, e.g. in Southern or Northern analysis, dot blot or other membrane-based technologies; in PCR technologies; or in dip stick, pin, ELISA or chip assays utilizing fluids or tissues from patient biopsies to detect altered gene expression.

In a particular aspect, the nucleotide sequences specific for the proteins of the invention and homologous nucleic acids may be useful in assays that detect activation or induction of various metabolic diseases or dysfunctions, including obesity, diabetes, and/or metabolic syndrome, as well as related disorders such as eating disorder, cachexia, hypertension, coronary heart disease, hypercholesterolemia, dyslipidemia, osteoarthritis, gallstones, or liver fibrosis. The nucleotide sequences may be labeled by standard methods, and added to a fluid or tissue sample from a patient under conditions suitable for the formation of hybridization complexes. After a suitable incubation period, the sample is washed and the signal is quantitated and compared with a standard value. If the amount of signal in the biopsied or extracted sample is significantly altered from that of a comparable have hybridized with nucleotide sequences in the sample, and the presence of altered levels of nucleotide sequences encoding the proteins of the invention and homologous proteins in the sample indicates the presence of the associated disease. Such assays may also be used to evaluate the efficacy of a particular therapeutic treatment regimen in animal studies, in clinical trials or in monitoring the treatment of an individual patient.

In order to provide a basis for the diagnosis of a disease associated with expression of the proteins of the invention and homologous proteins, a normal or standard profile for expression is established. This may be accomplished by combining body fluids or cell extracts taken from normal subjects, either animal or human, with a sequence or a fragment thereof, which is specific for the nucleic acids encoding the proteins of the invention and homologous nucleic acids, under conditions suitable for hybridization or amplification. Standard hybridization may be quantified by comparing the values obtained from normal subjects with those from an experiment where a known amount of a substantially purified polynucleotide is used. Standard values obtained from normal samples may be compared with values obtained from samples from patients who are symptomatic for disease. Deviation between standard and subject values is used to establish the presence of disease. Once disease is established and a treatment protocol is initiated, hybridization assays may be repeated on a regular basis to evaluate whether the level of expression in the patient begins to approximate that, which is observed in the normal patient. The results obtained from successive assays may be used to show the efficacy of treatment over a period ranging from several days to months.

With respect to metabolic diseases such as described above the presence of an unusual amount of transcript in biopsied tissue from an individual may indicate a predisposition for the development of the disease or may provide a means for detecting the disease prior to the appearance of actual clinical symptoms. A more definitive diagnosis of this type may allow health professionals to employ preventative measures or aggressive treatment earlier thereby preventing the development or further progression of the metabolic diseases and disorders.

Additional diagnostic uses for oligonucleotides designed from the sequences encoding the proteins of the invention and homologous proteins may involve the use of PCR. Such oligomers may be chemically synthesized, generated enzymatically or produced from a recombinant source. Oligomers will preferably consist of two nucleotide sequences, one with sense orientation (5′.fwdarw.3′) and another with antisense (3′.rarw.5′), employed under optimized conditions for identification of a specific gene or condition. The same two oligomers, nested sets of oligomers or even a degenerate pool of oligomers may be employed under less stringent conditions for detection and/or quantification of closely related DNA or RNA sequences.

In another embodiment of the invention, the nucleic acid sequences may also be used to generate hybridization probes, which are useful for mapping the naturally occurring genomic sequence. The sequences may be mapped to a particular chromosome or to a specific region of the chromosome using well known techniques. Such techniques include FISH, FACS or artificial chromosome constructions, such as yeast artificial chromosomes, bacterial artificial chromosomes, bacterial P1 constructions or single chromosome cDNA libraries as reviewed in Price C. M., (1993) Blood Rev. 7: 127-134, and Trask B. J., (1991) Trends Genet. 7: 149-154. FISH (as described in Verma R. S. and Babu A., (1988) Human Chromosomes: A Manual of Basic Techniques, Pergamon Press, New York, N.Y.). The results may be correlated with other physical chromosome mapping techniques and genetic map data. Examples of genetic map data can be found in the 1994 Genome Issue of Science (265: 1981f). Correlation between the location of the gene encoding the proteins of the invention on a physical chromosomal map and a specific disease or predisposition to a specific disease, may help to delimit the region of DNA associated with that genetic disease.

The nucleotide sequences of the subject invention may be used to detect differences in gene sequences between normal, carrier or affected individuals. In situ hybridization of chromosomal preparations and physical mapping techniques such as linkage analysis using established chromosomal markers may be used for extending genetic maps. Often the placement of a gene on the chromosome of another mammalian species, such as mouse, may reveal associated markers even if the number or arm of a particular human chromosome is not known. New sequences can be assigned to chromosomal arms or parts thereof, by physical mapping. This provides valuable information to investigators searching for disease genes using positional cloning or other gene discovery techniques. Once the disease or syndrome has been crudely localized by genetic linkage to a particular genomic region, for example, AT to 11 q22-23 (Gatti R. A. et al., (1988) Nature 336: 577-580), any sequences mapping to that area may represent associated or regulatory genes for further investigation. The nucleotide sequences of the subject invention may also be used to detect differences in the chromosomal location due to translocation, inversion, etc. among normal, carrier or affected individuals.

In another embodiment of the invention, the proteins of the invention, their catalytic or immunogenic fragments or oligopeptides thereof, an in vitro model, a genetically altered cell or animal, can be used for screening libraries of compounds, e.g. peptides or low molecular weight organic compounds, in any of a variety of drug screening techniques. One can identify modulators/effectors, e.g. receptors, enzymes, proteins, ligands, or substrates that bind to, modulate or mimic the action of one or more of the proteins of the invention. The protein or fragment thereof employed in such screening may be free in solution, affixed to a solid support, borne on a cell surface, or located intracellularly. The formation of binding complexes, between the protein and the agent tested, may be measured. Agents could also, either directly or indirectly, influence the activity of the proteins of the invention.

In vivo, the enzymatic kinase or phosphatase activity of the unmodified polypeptides of CG30346, Mekk1, or Ady43A homologous kinases or protein phosphatase 2 towards a substrate can be measured. Activation of the kinase or phosphatase may be induced in the natural context by extracellular or intracellular stimuli, such as signaling molecules or environmental influences. One may generate a system containing CG30346, Mekk1, or Ady43A homologous kinases or protein phosphatase 2, may it be an organism, a tissue, a culture of cells or cell-free environment, by exogenously applying this stimulus or by mimicking this stimulus by a variety of the techniques, some of them described further below. A system containing activated CG30346, Mekk1, or Ady43A homologous kinases or protein phosphatase 2 may be produced (i) for the purpose of diagnosis, study, prevention, and treatment of diseases and disorders related to body-weight regulation and thermogenesis, for example, but not limited to, metabolic diseases, (ii) for the purpose of identifying or validating therapeutic candidate agents, pharmaceuticals or drugs that influence the genes of the invention or their encoded polypeptides, (iii) for the purpose of generating cell lysates containing activated polypeptides encoded by the genes of the invention, (iv) for the purpose of isolating from this source activated polypeptides encoded by the genes of the invention.

In one embodiment of the invention, one may produce activated CG30346, Mekk1, or Ady43A homologous kinases or protein phosphatase 2 independent of the natural stimuli for the above said purposes by, for example, but not limited to, (i) an agent that mimics the natural stimulus; (ii) an agent, that acts downstream of the natural stimulus, such as activators of CG30346, Mekk1, or Ady43A homologous kinases or protein phosphatase 2, constitutive active alleles of CG30346, Mekk1, or Ady43A homologous kinases or protein phosphatase 2 themself as they are described or may be developed; (iii) by introduction of single or multiple amino acid substitutions, deletions or insertions within the sequence of CG30346, Mekk1, or Ady43A homologous kinases or protein phosphatase 2 to yield constitutive active forms; (iv) by the use of isolated fragments of CG30346, Mekk1, or Ady43A homologous kinases or protein phosphatase 2. In addition, one may generate enzymatically active CG30346, Mekk1, or Ady43A homologous kinases or protein phosphatase 2 in an ectopic system, prokaryotic or eukaryotic, in vivo or in vitro, by co-transfering to this system the activating components.

In addition activity of guf, CG3811, CG30346 (CG8080), rdgB, Mekk1, Ady43A, CG14816, tws, and PP2A-B′ homologous proteins against their physiological substrate(s) or derivatives thereof could be measured in cell-based or cell-free assays. Agents may also interfere with posttranslational modifications of the proteins of the invention, such as phosphorylation and dephosphorylation, farnesylation, palmitoylation, acetylation, alkylation, ubiquitination, proteolytic processing, subcellular localization and degradation. Moreover, agents could influence the dimerization or oligomerization of the proteins of the invention or, in a heterologous manner, of the proteins of the invention with other proteins, for example, but not exclusively, docking proteins, enzymes, receptors, ion channels, uncoupling proteins, or translation factors. Agents could also act on the physical interaction of the proteins of this invention with other proteins, which are required for protein function, for example, but not exclusively, their downstream signaling.

Methods for determining protein-protein interaction are well known in the art. For example binding of a fluorescently labeled peptide derived from a protein of the invention to the interacting protein (or vice versa) could be detected by a change in polarisation. In case that both binding partners, which can be either the full length proteins as well as one binding partner as the full length protein and the other just represented as a peptide are fluorescently labeled, binding could be detected by fluorescence energy transfer (FRET) from one fluorophore to the other. In addition, a variety of commercially available assay principles suitable for detection of protein-protein interaction are well known In the art, for example but not exclusively AlphaScreen (PerkinElmer) or Scintillation Proximity Assays (SPA) by Amersham. Alternatively, the interaction of the proteins of the invention with cellular proteins could be the basis for a cell-based screening assay, in which both proteins are fluorescently labeled and interaction of both proteins is detected by analysing cotranslocation of both proteins with a cellular imaging reader, as has been developed for example, but not exclusively, by Cellomics or EvotecOAI In all cases the two or more binding partners can be different proteins with one being the protein of the invention, or in case of dimerization and/or oligomerization the protein of the invention itself. Proteins of the invention, for which one target mechanism of interest, but not the only one, would be such protein/protein interactions are guf, CG3811, CG30346 (CG8080), rdgB, Mekk1, Ady43A, CG14816, tws, and PP2A-B′.

Target mechanisms could for example include the transporter or other conductance activities of the proteins of the invention as well as the regulation of the activity of the proteins of the invention by phosphorylation and dephosphorylation or other posttranslational modifications.

Assays for determining enzymatic, carrier, or ion channel activity of the proteins of the invention are well known in the art. Well known in the art are also a variety of assay formats to measure receptor-ligand binding.

Of particular interest are screening assays for agents that have a low toxicity for mammalian cells. The term “agent” as used herein describes any molecule, e.g. protein or pharmaceutical, with the capability of altering or mimicking the physiological function of one or more of the proteins of the invention. Candidate agents encompass numerous chemical classes, though typically they are organic molecules, preferably small organic compounds having a molecular weight of more than 50 and less than about 2,500 Daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise carbocyclic or heterocyclic structures and/or aromatic or polyaromatic structures substituted with one or more of the above functional groups.

Candidate agents are also found among biomolecules including peptides, saccharides, fatty acids, steroids, purines, pyrimidines, nucleic acids and derivatives, structural analogs or combinations thereof. Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptdes. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Where the screening assay is a binding assay, one or more of the molecules may be joined to a label, where the label can directly or indirectly provide a detectable signal.

Candidate agents may also be found in kinase or phosphatase assays where a kinase/phosphatase substrate such as a protein, a peptide, a lipid, or an organic compound, which may or may not include modifications as further described below, or others are phosphorylated or dephosphorylated by the proteins or protein fragments of the invention. The kinase or phosphatase can be a protein of the invention (e.g. CG30346, Mekk1, or Ady43A homologous kinases) or a kinase or phosphatase which is influenced in its activity by a protein of the invention (e.g. tws or PP2A-B′ homologous regulatory subunits of protein phosphatase 2). A therapeutic candidate agent may be identified by its ability to increase or decrease the enzymatic activity of the proteins of the invention. The kinase or phosphatase activity may be detected by change of the chemical, physical or immunological properties of the substrate due to phosphorylation or dephosphorylation. One example could be the transfer of radioisotopically labelled phosphate groups from an appropriate donor molecule to the kinase substrate catalyzed by the polypeptides of the invention or the cleavage from the phosphatase substrate by the phosphatase. The phosphorylation or dephosphorylation of the substrate may be followed by detection of the substrates autoradiography with techniques well known in the art.

Yet in another example, the change of mass of the substrate due to its phosphorylation or dephosphorylation may be detected by mass spectrometry techniques. One could also detect the phosphorylation status of a substrate with an analyte discriminating between the phosphorylated and unphosphorylated status of the substrate. Such an analyte may act by having different affinities for the phosphorylated and unphosphorylated forms of the substrate or by having specific affinity for phosphate groups. Such an analyte could be, but is not limited to, an antibody or antibody derivative, a recombinant antibody-like structure, a protein, a nucleic acid, a molecule containing a complexed metal ion, an anion exchange chromatography matrix, an affinity chromatography matrix or any other molecule with phosphorylation dependent selectivity towards the substrate.

Such an analyte could be employed to detect the kinase or phosphatase substrate, which is immobilized on a solid support during or after an enzymatic reaction. If the analyte is an antibody, its binding to the substrate could be detected by a variety of techniques as they are described in Harlow and Lane, 1998, Antibodies, CSH Lab Press, NY. If the analyte molecule is not an antibody, it may be detected by virtue of its chemical, physical or immunological properties, being endogenously associated with it or engineered to it.

Yet in another example the kinase or phosphatase substrate may have features, designed or endogenous, to facilitate its binding or detection in order to generate a signal that is suitable for the analysis of the substrates phosphorylation status. These features may be, but are not limited to, a biotin molecule or derivative thereof, a glutathione-S-transferase moiety, a moiety of six or more consecutive histidine residues, an amino acid sequence or hapten to function as an epitope tag, a fluorochrome, an enzyme or enzyme fragment. The kinase substrate may be linked to these or other features with a molecular spacer arm to avoid steric hindrance.

In one example, the kinase substrate may be labelled with a fluorochrome. The binding of the analyte to the labelled substrate in solution may be followed by the technique of fluorescence polarization as it is described in the literature (see, for example, Deshpande, S. et al., (1999) Prog. Biomed. Optics (SPIE) 3603: 261; Parker G. J. et al., (2000) J. Biomol. Screen. 5: 77-88; Wu, P. et al. (1997) Anal. Biochem. 249: 29-36). In a variation of this example, a fluorescent tracer molecule may compete with the substrate for the analyte to detect kinase activity by a technique which is known to those skilled in the art as indirect fluorescence polarization.

Another technique for drug screening, which may be used, provides for high throughput screening of compounds having suitable binding affinity to the protein of interest as described in published PCT application WO84/03564. In this method, as applied to the proteins of the invention large numbers of different small test compounds are synthesised on a solid substrate, such as plastic pins or some other surface. The test compounds are reacted with a protein of the invention, or fragments thereof, and washed. Bound proteins are then detected by methods well known in the art. Purified proteins can also be coated directly onto plates for use in the aforementioned drug screening techniques. Alternatively, non-neutralizing antibodies can be used to capture the peptide and immobilise it on a solid support.

In another embodiment, one may use competitive drug screening assays in which neutralising antibodies capable of binding guf, CG3811, CG30346 (CG8080), rdgB, Mekk1, Ady43A, CG14816, tws, and PP2A-B′ homologous protein specifically compete with a test compound for binding said protein. In this manner, the antibodies can be used to detect the presence of any peptide, which shares one or more antigenic determinants with guf, CG3811, CG30346 (CG8080, rdgB, Mekk1, Ady43A, CG14816, tws, and PP2A-B′.

The nucleic acids encoding a protein of the invention can be used to generate transgenic animals or site-specffic gene modifications in cell lines. These transgenic non-human animals are useful in the study of the function and regulation of said protein in vivo. Transgenic animals, particularly mammalian transgenic animals, can serve as a model system for the investigation of many developmental and cellular processes common to humans. A variety of non-human models of metabolic disorders can be used to test modulators/effectors of the proteins of the invention. Misexpression (for example, overexpression or lack of expression) of a protein of the invention, particular feeding conditions, or/and administration of biologically active compounds can create models of metablic disorders.

In one embodiment of the invention, such assays use mouse models of insulin resistance or/and diabetes, such as mice carrying gene knockouts in the leptin pathway (for example, ob (leptin) or db (leptin receptor) mice). Such mice develop typical symptoms of diabetes, show hepatic lipid accumulation and frequently have increased plasma lipid levels (see Bruning J. C. et al, (1998) Mol. Cell. 2: 559-569). Susceptible wild type mice (for example C57BI/6) show similiar symptoms if fed a high fat diet. In addition to testing the expression of the proteins of the invention in such mouse strains (see Examples section), these mice could be used to test whether administration of a candidate modulator/effector alters for example lipid accumulation in the liver, in plasma, or adipose tissues using standard assays well known in the art, such as FPLC, colorimetric assays, blood glucose level tests, insulin tolerance tests and others.

Transgenic animals may be made through homologous recombination in non-human embryonic stem cells, where the normal locus of the gene encoding a protein of the invention is altered. Alternatively, a nucleic acid construct encoding a protein of the invention is injected into oocytes and is randomly integrated into the genome. Vectors for stable integration include plasmids, retroviruses and other animal viruses, yeast artificial chromosomes (YACs), and the like. The modified cells or animal are useful in the study of the function and regulation of the proteins of the invention. For example, a series of small deletions or/and substitutions may be made in the gene that encodes a protein of the invention to determine the role of particular domains of the protein, functions in pancreatic differentiation, etc.

Furthermore, variants of the genes of the invention like specific constructs of interest include anti-sense molecules, which will block the expression of the proteins of the invention, or expression of dominant negative mutations. A detectable marker, such as for example lac-Z or lupiferase may be introduced in the locus of a gene of the invention, where up regulation of expression of the genes of the invention will result in an easily detected change in phenotype.

One may also provide for expression of the genes of the invention or variants thereof in cells or tissues where it is not normally expressed or at abnormal times of development. In addition, by providing expression of the proteins of the invention in cells in which they are not normally produced, one can induce changes in cell behavior.

DNA constructs for homologous recombination will comprise at least portions of the genes of the invention with the desired genetic modification, and will include regions of homology to the target locus. DNA constructs for random integration do not need to contain regions of homology to mediate recombination. Conveniently, markers for positive and negative selection are included. DNA constructs for random integration will consist of the nucleic acids encoding the proteins of the invention, a regulatory element (promoter), an intron and a poly-adenylation signal. Methods for generating cells having targeted gene modifications through homologous recombination are known in the art. For non-human embryonic stem (ES) cells, an ES cell line may be employed, or embryonic cells may be obtained freshly from a host, e.g. mouse, rat, guinea pig, etc. Such cells are grown on an appropriate fibroblast-feeder layer and are grown in the presence of leukemia inhibiting factor (LIF).

When non-human ES or embryonic cells or somatic pluripotent stem cells have been transfected, they may be used to produce transgenic animals. After transfection, the cells are plated onto a feeder layer in an appropriate medium.

Cells containing the construct may be selected by employing a selective medium. After sufficient time for colonies to grow, they are picked and analyzed for the occurrence of homologous recombination or integration of the construct. Those colonies that are positive may then be used for embryo transfection and morula aggregation. Briefly, morulae are obtained from 4 to 6 week old superovulated females, the Zona Pellucida is removed and the morulae are put into small depressions of a tissue culture dish. The ES cells are trypsinized, and the modified cells are placed into the depression closely to the morulae. On the following day the aggregates are transfered into the uterine horns of pseudopregnant females. Females are then allowed to go to term. Chimeric offsprings can be readily detected by a change in coat color and are subsequently screened for the transmission of the mutation into the next generation (F1-generation). Offspring of the F1-generation are screened for the presence of the modified gene and males and females having the modification are mated to produce homozygous progeny. If the gene alterations cause lethality at some point in development, tissues or organs can be maintained as allogenic or congenic grafts or transplants, or in vitro culture. The transgenic animals may be any non-human mammal, such as laboratory animal, domestic animals, etc., for example, mouse, rat, guinea pig, sheep, cow, pig, and others. The transgenic animals may be used in functional studies, drug screening, and other applications and are useful in the study of the function and regulation of the proteins of the invention in vivo.

Finally, the invention also relates to a kit comprising at least one of
(a) a nucleic acid molecule coding for a protein of the invention or a functional fragment thereof;
(b) a protein of the invention or a functional fragment or an isoform thereof;
(c) a vector comprising the nucleic acid of (a);
(d) a host cell comprising the nucleic acid of (a) or the vector of (c);
(e) a polypeptide encoded by the nucleic acid of (a);
(f) a fusion polypeptide encoded by the nucleic acid of (a);
(g) an antibody, an aptamer or another modulator/effector of the nucleic acid of (a) or the polypeptide of (b), (e), or (f) and
(h) an anti-sense oligonucleotide of the nucleic acid of (a).

The kit may be used for diagnostic or therapeutic purposes or for screening applications as described above. The kit may further contain user instructions.

### THE FIGURES SHOW

FIG. 1 shows the triglyceride content of a Drosophila guf (GadFly Accession Number CG16747) mutant. Shown is the change of triglyceride content of HD-EP(2)20697 flies caused by integration of the P-vector into the annotated transcription unit (referred to as ‘HD-EP20697’ in column 2) in comparison to controls containing all flies of the EP collection (referred to as ‘EP-control’ in column 1).

FIG. 2 shows the molecular organization of the mutated guf gene locus.

FIG. 3 shows the expression of guf homologs in mammalian (mouse) tissues.

FIG. 3A shows the real-time PCR analysis of ornithine decarboxylase antizyme 1 (Oaz1) expression in wild-type mouse tissues.

FIG. 3B shows the real-time PCR analysis of Oaz1expression in different mouse models.

FIG. 3C shows the real-time PCR analysis of Oaz1expression in mice fed with a high fat diet compared to mice fed with a control diet.

FIG. 3D shows the real-time PCR analysis of Oaz1expression during the differentiation of 3T3-L1 cells from preadipocytes to mature adipocytes.

FIG. 3E shows the real-time PCR analysis of ornithine decarboxylase antizyme 2 (Oaz2) expression in wild-type mouse tissues.

FIG. 3F shows the real-time PCR analysis of Oaz2 expression in different mouse models.

FIG. 3G shows the real-time PCR analysis of Oaz2 expression in mice fed with a high fat diet compared to mice fed with a control diet.

FIG. 3H shows the real-time PCR analysis of Oaz2 expression during the differentiation of 3T3-L1 cells from preadipocytes to mature adipocytes.

FIG. 3I shows the real-time PCR analysis of ornithine decarboxylase antizyme 3 (Oaz3) expression in wild-type mouse tissues.

FIG. 3J shows the real-time PCR analysis of Oaz3 expression in different mouse models.

FIG. 3K shows the real-time PCR analysis of Oaz3 expression in mice fed with a high fat diet compared to mice fed with a control diet.

FIG. 4 shows the triglyceride content of a Drosophila CG3811 (GadFly Accession Number) mutant. Shown is the change of triglyceride content of HD-EP(2)26828 flies caused by integration of the P-vector into the annotated transcription unit (referred to as ‘HD-EP26828’ in column 2) in comparison to controls containing all flies of the EP collection (referred to as ‘EP-control’ in column 1).

FIG. 5 shows the molecular organization of the mutated CG3811 gene locus.

FIG. 6 shows the expression of the CG3811 homolog in mammalian (mouse) tissues.

FIG. 6A shows the real-time PCR analysis of the protein similar to organic anion transporter polypeptide-related protein 4 (LOC240726) expression in wild-type mouse tissues.

FIG. 6B shows the real-time PCR analysis of LOC240726 expression in different mouse models.

FIG. 6C shows the real-time PCR analysis of LOC240726 expression in mice fed with a high fat diet compared to mice fed with a control diet.

FIG. 7 shows the triglyceride content of a Drosophila CG30346 (GadFly Accession Number) mutant. Shown is the change of triglyceride content of HD-EP(2)26046 flies caused by integration of the P-vector into the annotated transcription unit (referred to as ‘HD-EP26046’ in column 2) in comparison to controls containing all flies of the EP collection (referred to as ‘EP-control’ in column 1).

FIG. 8 shows the molecular organization of the mutated CG30346 gene locus.

FIG. 9 shows the expression of the CG30346 homolog in mammalian (mouse) tissues.

FIG. 9A shows the real-time PCR analysis of RIKEN cDNA 111 0020G09 gene (111002OG09Rik) expression in wild-type mouse tissues.

FIG. 9B shows the real-time PCR analysis of 111002OG09Rik expression in different mouse models.

FIG. 9C shows the real-time PCR analysis of 111002OG09Rik expression during the differentiation of 3T3-L1 cells from preadipocytes to mature adipocytes.

FIG. 10 shows the triglyceride content of a Drosophila retinal degeneration B (rdgB, GadFly Accession Number CG11111) mutant. Shown is the change of triglyceride content of HD-EP(X)11173 flies caused by integration of the P-vector into the annotated transcription unit (referred to as ‘HD-EP1 1173’ in column 2) in comparison to controls containing all flies of the EP collection (referred to as ‘EP-control’ in column 1).

FIG. 11 shows the molecular organization of the mutated rdgB gene locus.

FIG. 12 shows the comparison (CLUSTAL W 1.82 multiple sequence alignment) of human phosphatidylinositol transfer protein, human PYK2 N-terminal domain-interacting receptor 3, human PYK2 N-terminal domain-interacting receptor 1, and Drosophila retinal degeneration B protein. Gaps in the alignment are represented as. ‘NIR3_Hs’ refers to human PYK2 N-terminal domain-interacting receptor 3 (NIR3, NCBI LocusID: 57605), ‘NIR1_Hs’ refers to human PYK2 N-terminal domain-interacting receptor 1 (NIR1, NCBI LocusID: 83394), ‘PITPNM_Hs’ refers to human membrane-associated phosphatidylinositol transfer protein (PITPNM, similar to PYK2 N-terminal domain-interacting receptor 2 (NIR2), NCBI LocusID: 9600), and ‘CG11111-PB’ refers to the Drosophila rdgB protein.

FIG. 13 shows the expression of rdgB homologs in mammalian (mouse) tissues.

FIG. 13A shows the real-time PCR analysis of phosphatidylinositol membrane-associated (Pitpnm) expression in wild-type mouse tissues.

FIG. 13B shows the real-time PCR analysis of Pitpnm expression in different mouse models.

FIG. 13C shows the real-time PCR analysis of Pitpnm expression in mice fed with a high fat diet compared to mice fed with a control diet.

FIG. 13D shows the real-time PCR analysis of Pitpnm expression during the differentiation of 3T3-L1 cells from preadipocytes to mature adipocytes.

FIG. 13E shows the real-time PCR analysis of retinal degeneration B2 homolog (Drosophila) (Rdgb2) expression in wild-type mouse tissues.

FIG. 13F shows the real-time PCR analysis of Rdgb2 expression in different mouse models.

FIG. 13G shows the real-time PCR analysis of Rdgb2 expression during the differentiation of 3T3-L1 cells from preadipocytes to mature adipocytes.

FIG. 13H shows the real-time PCR analysis of similar to PYK2 N-terminal domain-interacting receptor 1 (LOC216884) expression in wild-type mouse tissues.

FIG. 13I shows the real-time PCR analysis of LOC216884 expression in different mouse models.

FIG. 13J shows the real-time PCR analysis of LOC216884 expression in mice fed with a high fat diet compared to mice fed with a control diet.

FIG. 14 shows the triglyceride content of a Drosophila Mekk1(GadFly Accession Number CG7717) mutant. Shown is the change of triglyceride content of HD-EP(3)31064 flies caused by integration of the P-vector into the annotated transcription unit (referred to as ‘HD-EP31064’ in column 2) in comparison to controls containing 2108 fly lines of the EP collection (referred to as ‘HD-EP collection (N=2108)’ in column 1).

FIG. 15 shows the molecular organization of the mutated Mekk1gene locus.

FIG. 16 shows the expression of a Mekk1homolog in mammalian (mouse) tissues.

FIG. 16A shows the real-time PCR analysis of mitogen activated protein kinase kinase kinase 4 (Map3k4) expression in wild-type mouse tissues.

FIG. 16B shows the real-time PCR analysis of Map3k4 expression in different mouse models.

FIG. 16C shows the real-time PCR analysis of Map3k4 expression during the differentiation of 3T3-L1 cells from preadipocytes to mature adipocytes.

FIG. 17 shows the triglyceride content and the glycogen content of a Drosophila Ady43A (GadFly Accession Number CG1851) mutant. Shown is the change of triglyceride content of HD-EP(2)21939 flies caused by integration of the P-vector into the annotated transcription unit (referred to as ‘HD-21939 (TG)’ in column 3) in comparison to controls containing more than 2000 fly lines of the proprietary EP collection (referred to as ‘HD-control (TG)’ in column 1) and wildtype controls determined in more than 80 independent assays (referred to as ‘WT-control (TG)’ in column 2). Also shown is the change of glycogen content of HD-EP(2)21939 flies caused by integration of the P-vector into the annotated transcription unit (referred to as ‘HD-21939 (glycogen)’, in column 5) in comparison to controls (referred to as ‘control (glycogen)’ in column 4).

FIG. 18 shows the molecular organization of the mutated Ady43A gene locus.

FIG. 19 shows the expression of Ady43A homologs in mammalian (mouse) tissues.

FIG. 19A shows the real-time PCR analysis of adenosine kinase (Adk) expression in wild-type mouse tissues.

FIG. 19B shows the real-time PCR analysis of Adk expression in different mouse models.

FIG. 19C shows the real-time PCR analysis of Adk expression in mice fed with a high fat diet compared to mice fed with a control diet.

FIG. 19D shows the real-time PCR analysis of Adk expression during the differentiation of 3T3-L1 cells from preadipocytes to mature adipocytes.

FIG. 20 shows the triglyceride content of a Drosophila CG14816 (GadFly Accession Number) mutant. Shown is the change of triglyceride content of HD-EP(X)11032 flies caused by integration of the P-vector into the annotated transcription unit (referred to as ‘HD-EP11032’ in column 2) in comparison to controls containing all flies of the EP collection (referred to as ‘EP-control’ in column 1).

FIG. 21 shows the molecular organization of the mutated CG14816 gene locus.

FIG. 22 shows the expression of the CG14816 homolog in mammalian (mouse) tissues.

FIG. 22A shows the real-time PCR analysis of RLKEN cDNA 2610528A17 gene (2610528A17Rik) expression in wild-type mouse tissues.

FIG. 22B shows the real-time PCR analysis of 2610528A17Rik expression in different mouse models.

FIG. 22C shows the real-time PCR analysis of 2610528A17Rik expression in mice fed with a high fat diet compared to mice fed with a control diet.

FIG. 22D shows the real-time PCR analysis of 2610528A17Rik expression during the differentiation of 3T3-L1 cells from preadipocytes to mature adipocytes.

FIG. 23 shows the triglyceride content of a Drosophila tws (GadFly Accession Number CG6235) mutant. Shown is the change of triglyceride content of HD-EP(3)31733/TM3,Ser flies caused by heterozygous integration of the P-vector into the annotated transcription unit (referred to as ‘HD-EP31 733/TM3, Ser’ in column 2) in comparison to controls containing all flies of the EP collection (referred to as ‘EP-control’ in column 1).

FIG. 24 shows the molecular organization of the mutated tws gene locus.

FIG. 25 shows the triglyceride content of a Drosophila PP2A-B′ (GadFly Accession Number CG7913) mutant. Shown is the change of triglyceride content of HD-EP(2)26046 flies caused by ectopic expression of the PP2A-B′ gene mainly in the neurons of these flies (referred to as ‘HD-EP(3)31721 /elav’ in column 2) in comparison to controls containing all flies of the EP/elav collection (referred to as ‘EP/elav control’ in column 1).

FIG. 26 shows the molecular organization of the mutated PP2A-B′ gene locus.

FIG. 27 shows the expression of human guf homologs in mammalian (human) tissue.

FIG. 27A shows the microarray analysis of ornithine decarboxylase antizyme 1 (OAZ1) expression in human abdominal derived primay adipocyte cells, during the differentiation from preadipocytes to mature adipocytes.

FIG. 27B shows the microarray analysis of ornithine decarboxylase antizyme 2 (OAZ2) expression in human abdominal derived primay adipocyte cells, during the differentiation from preadipocytes to mature adipocytes.

### THE EXAMPLES ILLUSTRATE THE INVENTION

### Example 1

### Measurement of Triglyceride and/or Glycogen Content in Drosophila

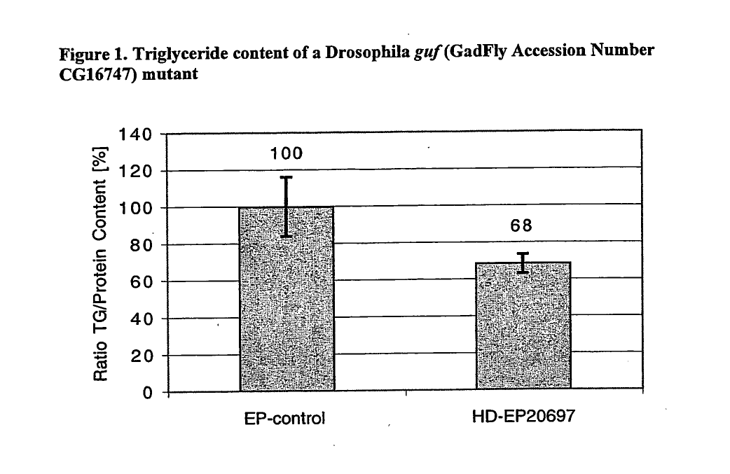

Mutant flies are obtained from a fly mutation stock collection. The flies are grown under standard conditions known to those skilled in the art. In the course of the experiment, additional feedings with bakers yeast (Saccharomyces cerevisiae) are provided. The average change of triglyceride and glycogen (herein referred to as energy storage metabolites, ESM) content of Drosophila containing the EP-vectors in homozygous viable, hemizygous viable, or homozygous lethal/heterozygous viable integration, was investigated in comparison to control flies grown under the same conditions (see FIGS. 1, 4, 7, 10, 14, 17, 20, 23, and 25). For determination of triglyceride and ESM content, flies were incubated for 5 min at 90° C. in an aqueous buffer using a waterbath, followed by hot extraction. After another 5 min incubation at 90° C. and mild centrifugation, the triglyceride content of the flies extract was determined using Sigma Triglyceride (INT 336−10 or −20) assay by measuring changes in the optical density according to the manufacturer's protocol. The glycogen content of the flies extract was determined using the Roche Starch UV-method (Cat. No. 0207748) assay by measuring changes in the optical density according to the manufacturer's protocol. As a reference the protein content of the same extract was measured using BIO-RAD DC Protein Assay according to the manufacturers protocol. These experiments and assays were repeated several times.

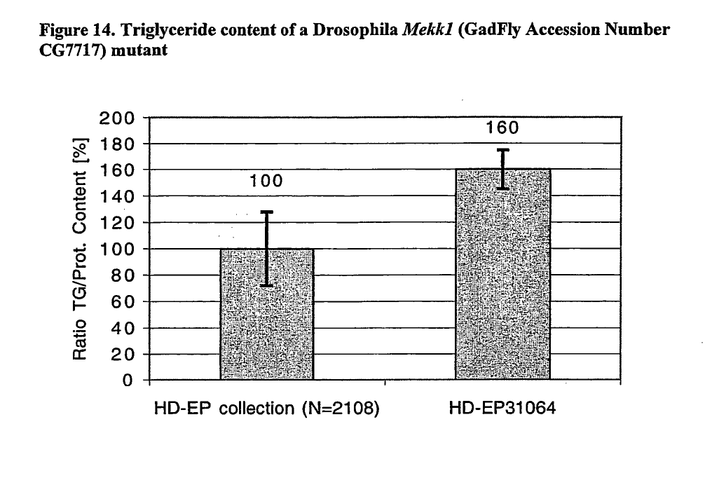

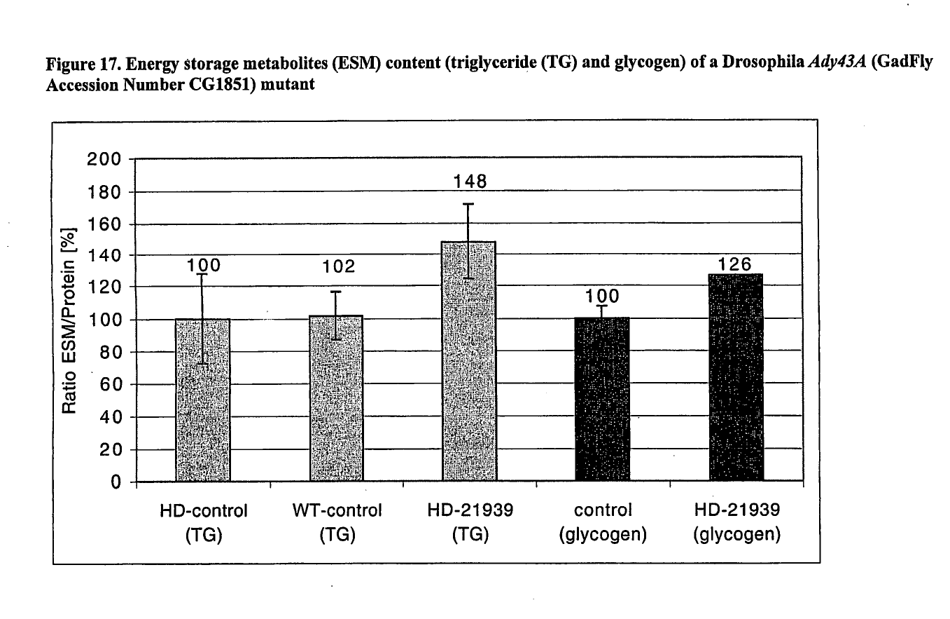

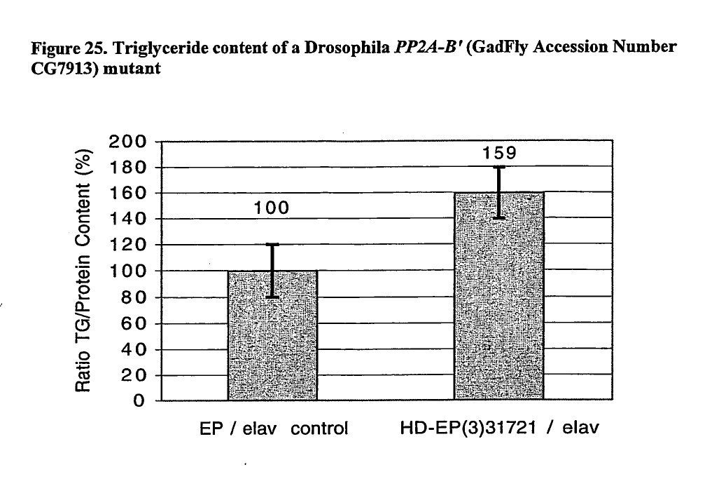

The average triglyceride level of all flies of the EP collection (referred to as ‘EP-control’) is shown as 100% in the first columns in FIGS. 1, 4, 7, 10, 20, and 23. The average triglyceride level of 2108 flies of the HD-EP collection (referred to as ‘HD-EP collection (N=2108)’) is shown as 100% in the first column in FIG. 14. The average triglyceride level (μg triglyceride/μg protein) of 2108 fly lines of the proprietary EP-collection (referred to as ‘HD-control (TG)’) is shown as 100% in the first column in FIG. 17. The average triglyceride level (μg triglyceride/μg protein) of wildtype OreR flies was determined in 84 independent assays (referred to as ‘Wr-control (TG)’) is shown as 102% in the second column in FIG. 17. The average glycogen level (μg glycogen/μg protein) of an internal assay control consisting of two different wildtype strains and an inconspicuous EP-line of the HD stock collection (referred to as ‘control (glycogen)’) is shown as 100% in the fourth column in FIG. 17. The average triglyceride level of all flies containing the elav-Gal4 vector (referred to as ‘EP/elav control’) is shown as 100% in the first column in FIG. 25. Standard deviations of the measurements are shown as thin bars.

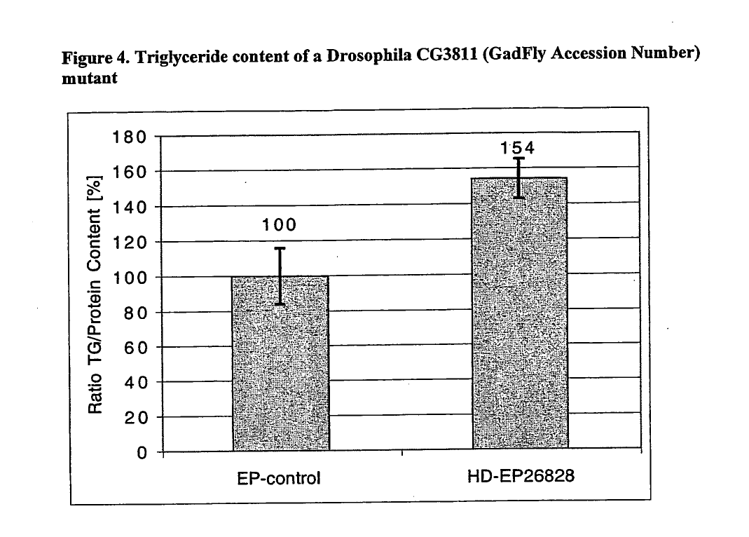

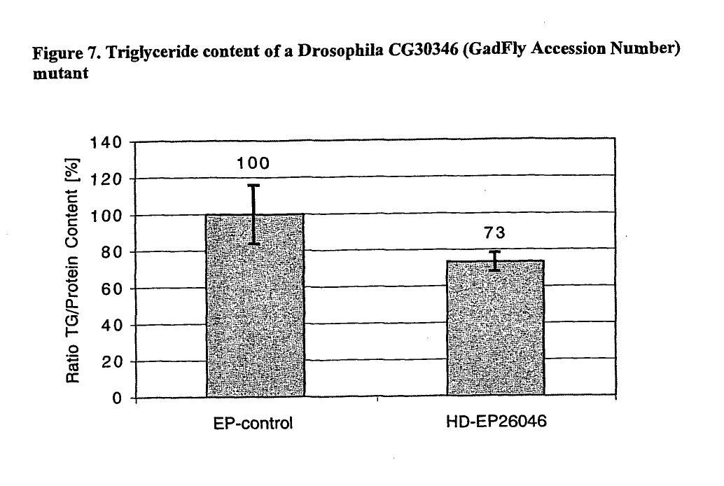

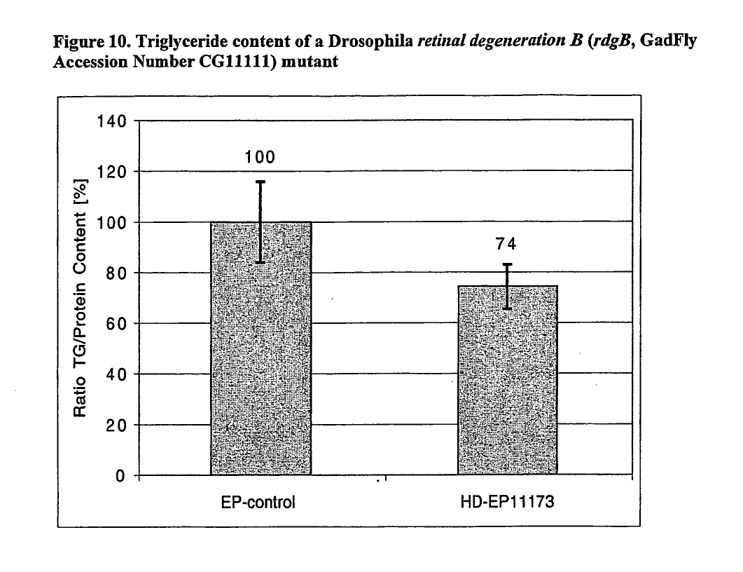

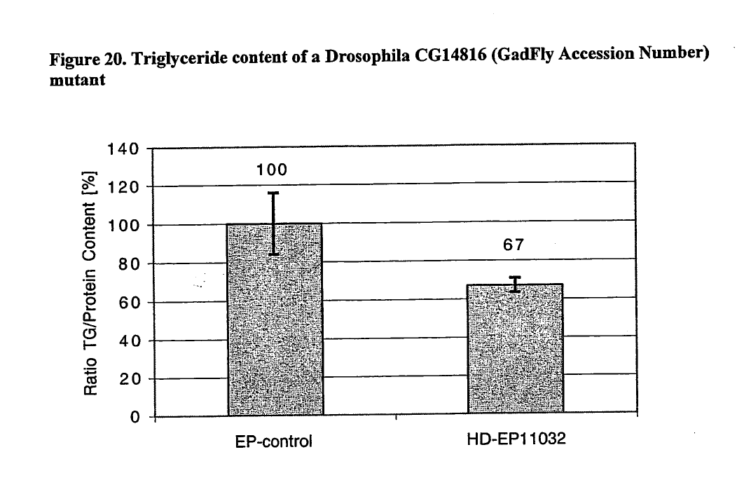

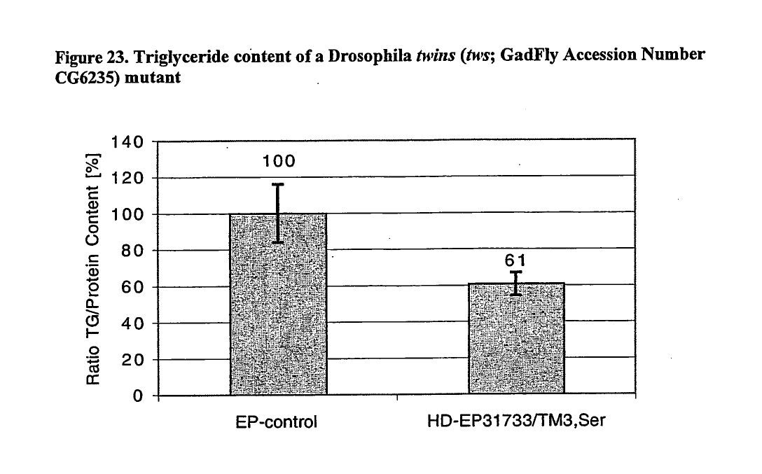

HD-EP(2)20697 homozygous flies (column 2 in FIG. 1, ‘HD-EP20697’), HD-EP(2)26046 homozygous flies (column 2 in FIG. 7, ‘HD-EP26046’), HD-EP(X)11032 homozygous flies (column 2 in FIG. 20, ‘HD-EP11032’), and HD-EP(3)31733 heterozygous flies, containing a TM3, Ser balancer chromosome (column 2 in FIG. 23, ‘HD-EP31733/TM3, Ser’) show constantly a lower triglyceride content than the controls. HD-EP(2)26828 homozygous flies (column 2 in FIG. 4, ‘HD-EP26828’), HD-EP(X) 1173 hemizygous flies (column 2 in FIG. 10, ‘HD-EP11173’), HD-EP(3)31064 homozygous flies (column 2 in FIG. 14, ‘HD-EP31064’), and HD-EP(2)21939 homozygous flies (column 3 in FIG. 17, ‘HD-21939 (TG)’) show constantly a higher triglyceride content than the controls. HD-21939 homozygous flies (column 5 in FIG. 17, ‘HD-21939 (glycogen)’) also show a higher glycogen content than the controls. Therefore, the loss of gene activity in the loci, where the EP-vectors are integrated, are responsible for changes in the metabolism of the energy storage triglycerides.

The offspring of HD-EP(3)31721 males that are crossed to elav-Gal4 virgins, carrying a copy of the HD-EP(3)31721 vector and a copy of the elav-Gal4 vector, leading to ectopic expression of adjacent genomic DNA sequences 3′ of the HD-EP(3)31721 integration locus, mainly in neurons of these flies (column 2 in FIG. 25, ‘HD-EP(3)31721/elav’), shows constantly a higher triglyceride content than the controls. Therefore, the gain of gene activity in the neurons is responsible for changes in the metabolism of the energy storage triglycerides.

### Example 2

### Identification of Drosophila Genes Associated With Energy Homeostasis

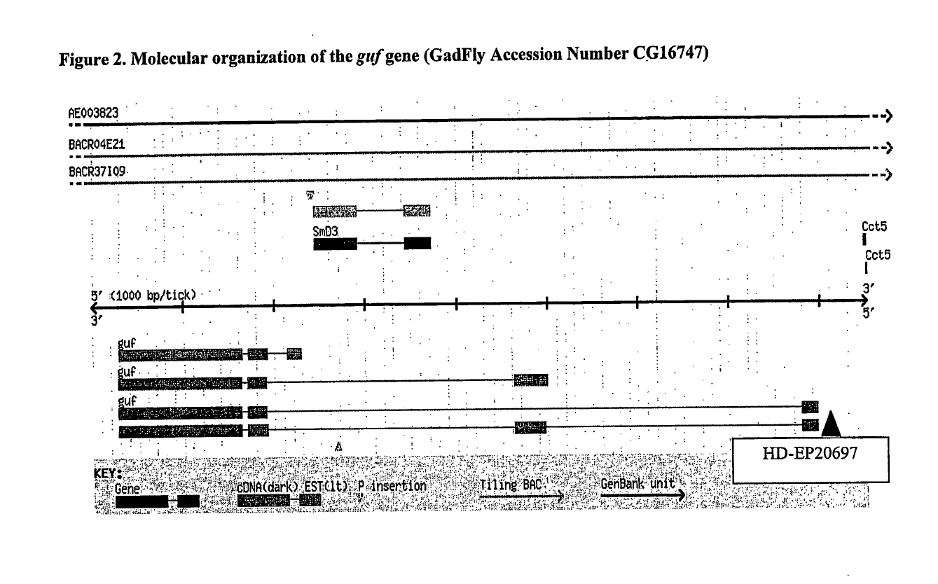

Nucleic acids encoding the proteins of the present invention were identified using a plasmid-rescue technique. Genomic DNA sequences were isolated that are localized adjacent to the EP vector (herein HD-EP(2)20697, HD-EP(2) 26828, HD-EP(2)26046, HD-EP(X)11173, HD-EP(3)31064, HD-EP(2)21939, HD-EP(X)11032, HD-EP(3)31733, and HD-EP(3)31721) integration. Using those isolated genomic sequences public databases like Berkeley Drosophila Genome Project (GadFly) were screened, thereby identifying the integration sites of the vectors, and the corresponding genes. The molecular organization of these gene loci is shown in FIGS. 2, 5, 8,11, 15, 18, 21, 24, and 26.

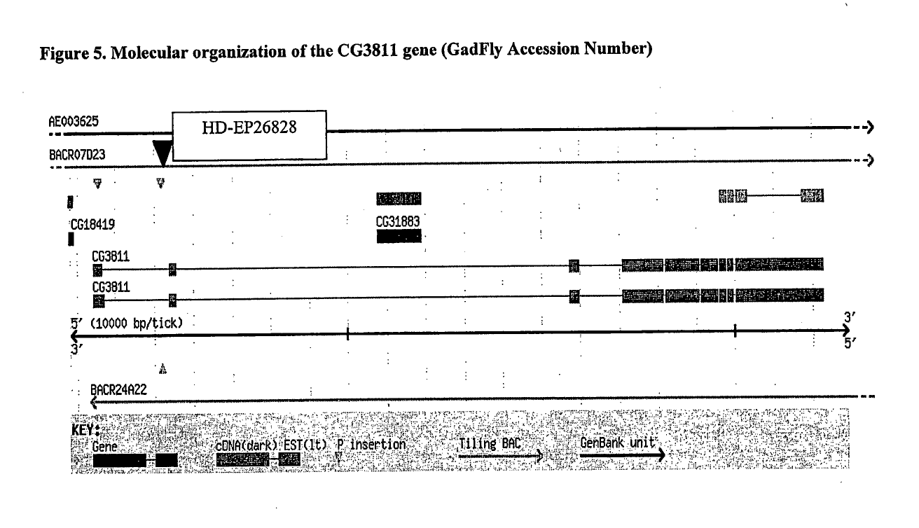

In FIGS. 2, 5, 8, 11, 15, 18, 24, and 26, genomic DNA sequence is represented by the assembly as a horizontal black scaled double-headed arrow that includes the integration sites of the vectors for lines HD-EP(2)20697, HD-EP(2)26828, HD-EP(2)26046, HD-EP(X)11173, HD-EP(3)31 064, HD-EP (2)21939, HD-EP(3)31733, and HD-EP(3)31721. Ticks represent the length in basepairs of the genomic DNA (1000 base pairs (FIGS. 2, 8, 15, 18, and 24) or 10000 base pairs (FIGS. 5, 11, and 26) per tick). The part of the figure above the arrow represents the sense strand, the part below the arrow represents the antisense strand. The grey arrows in the upper part and/or lower part of the figures represent BAC clones, the black arrows in the topmost part of the figures represent the sections of the chromosomes. The insertion sites of the P-elements in the Drosophila lines are shown as triangles and are labeled. The cDNA sequences of the predicted genes (as predicted by the Berkeley Drosophila Genome Project, GadFly and by Magpie) are shown as dark grey bars (exons), linked by dark grey lines (introns), and are labeled (see also key at the bottom of the figures).

The HD-EP(2)20697 vector is homozygous viable integrated 105 base pairs 5′ of a Drosophila gene, identified as CGI6747-RA, a transcript variant of Drosophila guf, in sense orientation. FIG. 2 shows the molecular organization of this gene locus. The chromosomal localization site of integration of the vector HD-EP(2)20697 is at gene locus 2R, 48E4. In FIG. 2, the insertion site of the P-element in Drosophila line HD-EP(2)20697 is labeled. The alternative splice variants of the Drosophila gene guf (GadFly Accession Number CG16747) are labeled.

The HD-EP(2)26828 vector is homozygous viable integrated into the first intron of CG3811-RA and CG3811-RB in sense orientation 5′ of the translation start. FIG. 5 shows the molecular organization of this gene locus. The chromosomal localization site of integration of the vector HD-EP(2)26828 is at gene locus 2L, 30B5 (according to FlyBase), 30B10 (according to GadFly release 3). In FIG. 5, the insertion site of the P-element in Drosophila HD-EP (2)26828 line is labeled. The predicted cDNAs of the Drosophila gene CG3811 (GadRy Accession Number) are labeled.

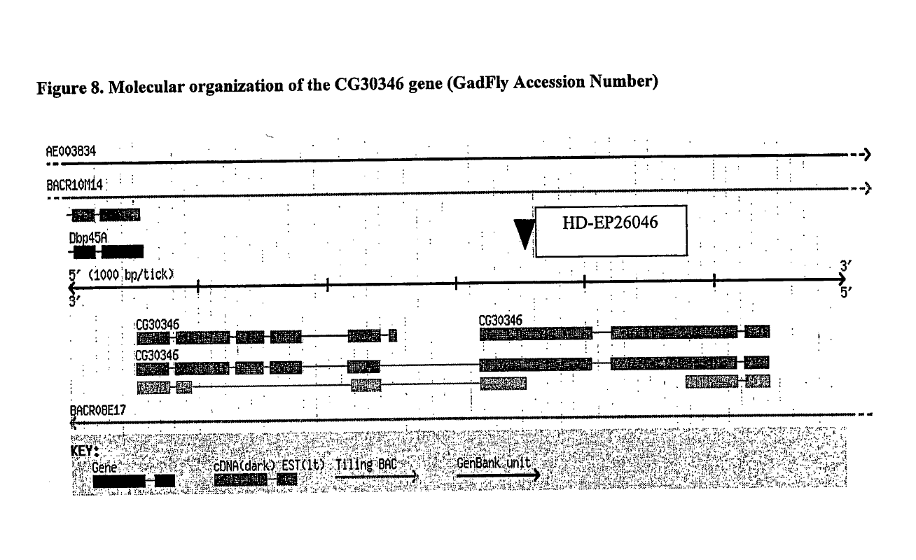

The HD-EP(2)26046) vector is homozygous viable integrated into the third exon of CG30346-RA and last exon of CG30346-RB in antisense orientation. FIG. 8 shows the molecular organization of this gene locus. The chromosomal localization site of integration of the vector HD-EP(2)26046 is at gene locus 2R2R, 45A2. In FIG. 8, the insertion site of the P-element in Drosophila line HD-EP(2)26046 is labeled. The predicted cDNAs of the Drosophila gene CG30346 (GadFly Accession Number) are labeled.

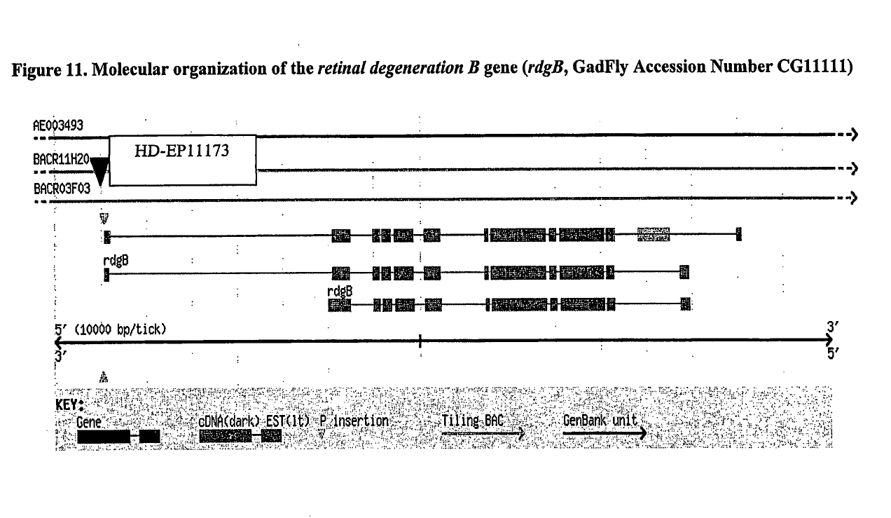

The HD-EP(X)11173 vector is hemizygous viable integrated 43 base pairs 5′ of expressed sequence tag (EST) GH09970, which overlaps with the cDNA of rdgB in sense orientation. FIG. 11 shows the molecular organization of this gene locus. The chromosomal localization site of integration of the vector of HD-EP(X)11173 is at gene locus X, 12C2 (according to Flybase), 12B8 (according to Gadfly release 2). In FIG. 11, the insertion site of the P-element in Drosophila HD-EP(X)11173 line is labeled. The predicted cDNAs of the Drosophila gene rdgB (GadFly Accession Number CG11111) are labeled.

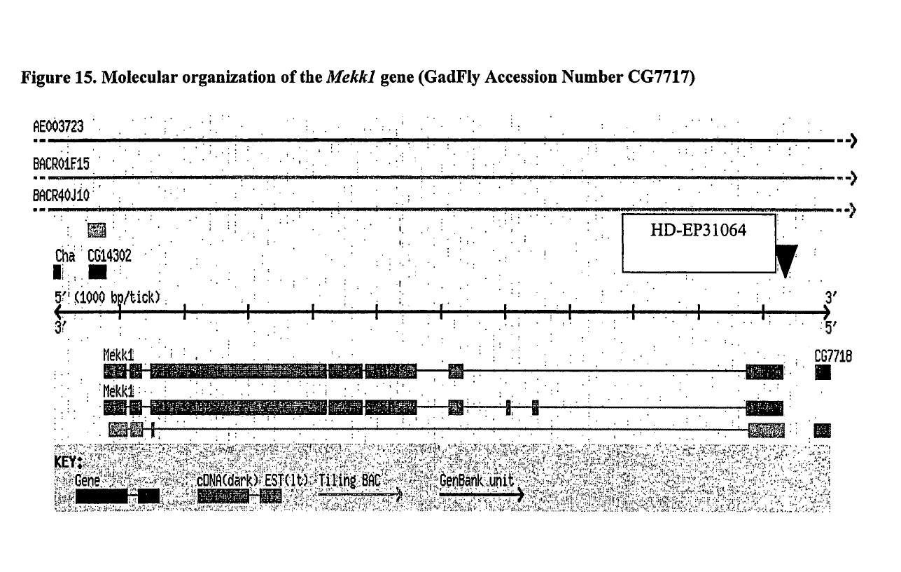

The HD-EP(3)31064 vector is homozygous viable integrated 13 base pairs 5′ of Mekk1in antisense orientation. FIG. 15 shows the molecular organization of this gene locus. The chromosomal localization site of integration of the vector HD-EP(3)31064 is at gene locus 3R, 91 B8-C1 (according to Flybase), 91 C5 (according to Gadfly release 3). In FIG. 15, the insertion site of the P-element in Drosophila line HD-EP(3)31064 is labeled. The predicted cDNAs of the Drosophila gene Mekk1(GadFly Accession Number CG7717) are labeled.

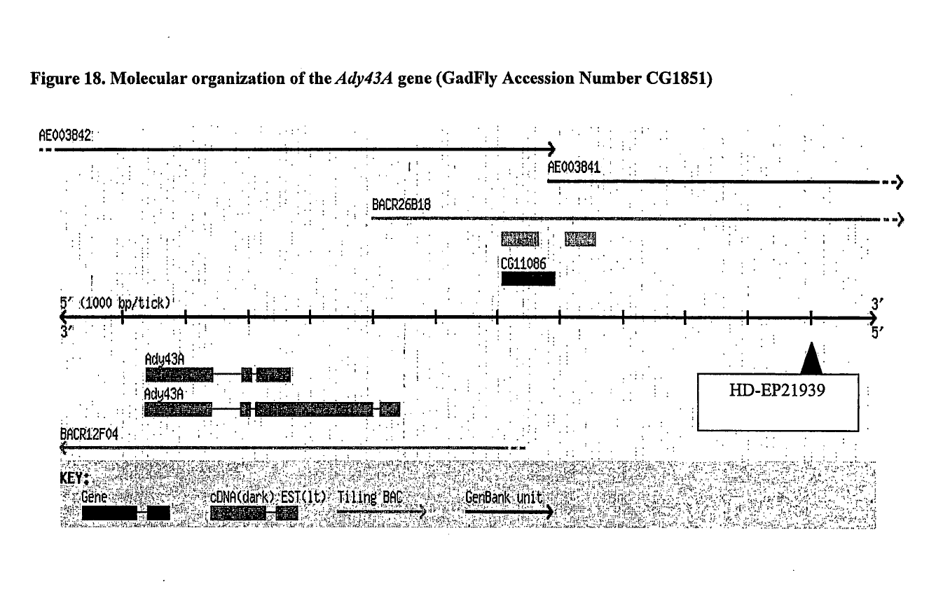

The HD-EP(2)21939 vector is homozygous viable integrated 6656 base pairs 5′ of the cDNA CG1851-RB in sense orientation. FIG. 18 shows the molecular organization of this gene locus. The chromosomal localization site of integration of the vector of HD-EP(2)21939 is at gene locus 2R, 43A2 (according to Flybase and Gadfly release 3). In FIG. 18, the insertion site of the P-element in Drosophila line HD-EP(2)21939 is labeled. The predicted cDNAs of the Drosophila gene Ady43A (GadFly Accession Number CG1851) are labeled.

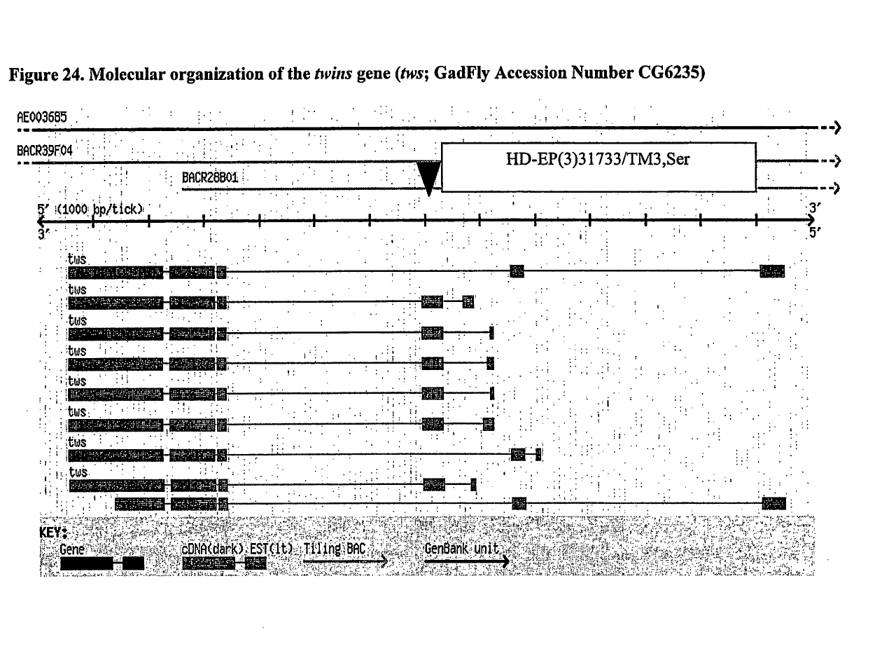

The HD-EP(3)31733 vector is homozygous lethal/heterozygous viable integrated into the second intron of CG6235-RA, and CG6235-RF, and into the second exon of CG6235-RB, CG6235-RC, CG6235-RD, CG6235-RE, CG6235-RG, and CG6235-RH in antisense orientation. FIG. 24 shows the molecular organization of this gene locus. The chromosomal localization site of integration of the vector HD-EP(3)31733 is at gene locus 3R, 85F10-12 (according to Flybase), 85F13-14 (according to Gadfly release 3). In FIG. 24, the insertion site of the P-element in Drosophila line HD-EP(3)31733 is labeled. The predicted cDNAs of the Drosophila gene tws (GadFly Accession Number CG6235) are labeled.

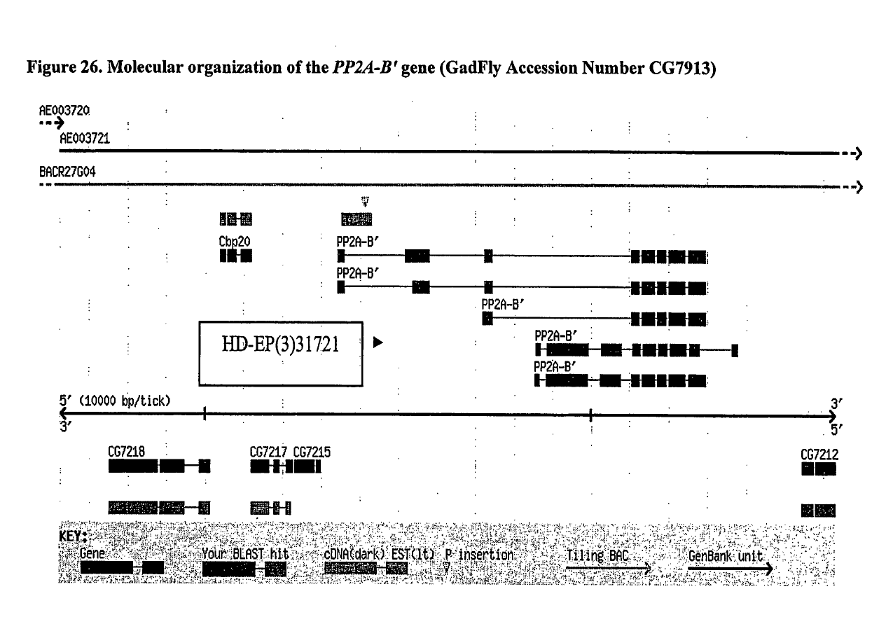

The HD-EP(3)31721 vector is homozygous viable integrated into the first intron of PP2A-B′ in sense orientation. FIG. 26 shows the molecular organization of this gene locus. The chromosomal localization site of integration of the vector HD-EP(3)31721 is at gene locus 3R, 90E3-4. In FIG. 26, the insertion site of the P-element in Drosophila line HD-EP(3)31721 is labeled. The predicted cDNAs of the Drosophila gene PP2A-B′ (GadFly Accession Number CG7913) are labeled.

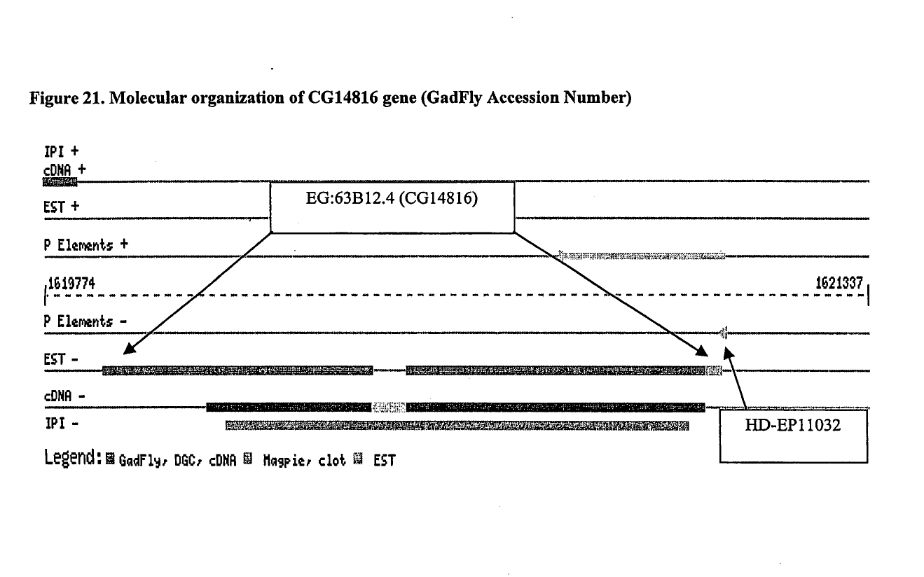

The HD-EP(X)11032 vector is hemizygous viable integrated in base pair 1 of the of expressed sequence tag (EST) SD17949 that overlaps with a Drosophila gene in sense orientation identified as GadFly Accession Number CG14816. FIG. 21 shows the molecular organization of this gene locus. The chromosomal localization site of integration of the vector HD-EP(X)11032 is at gene locus X, 2B13 (according to Flybase), 2A2 (according to Gadfly release 2). In FIG. 21, genomic DNA sequence is represented by the assembly as a dotted black line in the middle that includes the integration site of the vector for line HD-EP(X)11032. Numbers represent the coordinates of the genomic DNA (starting at position 1619774 on chromosome X, ending at position 1621337). The upper part of the figure represents the sense strand “+”, the lower part represents the antisense strand “−”. The insertion site of the P-element in the Drosophila line is shown as triangle in the “P-elements -” line and is labeled. Transcribed DNA sequences (ESTs) are shown as grey bars in the “EST -” and the “IPI -” lines, and predicted cDNAs are shown as bars in the “cDNA +” and “cDNA-” lines. Predicted exons of the cDNAs are shown as dark grey bars and predicted introns are shown as light grey bars (see also legend at the bottom of the figures). The predicted cDNA of the CG14816 gene is shown in the “cDNA ” line and the corresponding ESTs shown in the “EST -” and the “IPI-” lines are labeled (referred to as EG:63B12.4 (CG14816)).

Expression of the genes described above could be effected by integration of the vectors into the transcription units, leading to a change in the amount of the energy storage triglycerides and/or glycogen.

### Example 3

### Identification of Human Homologous Genes and Proteins

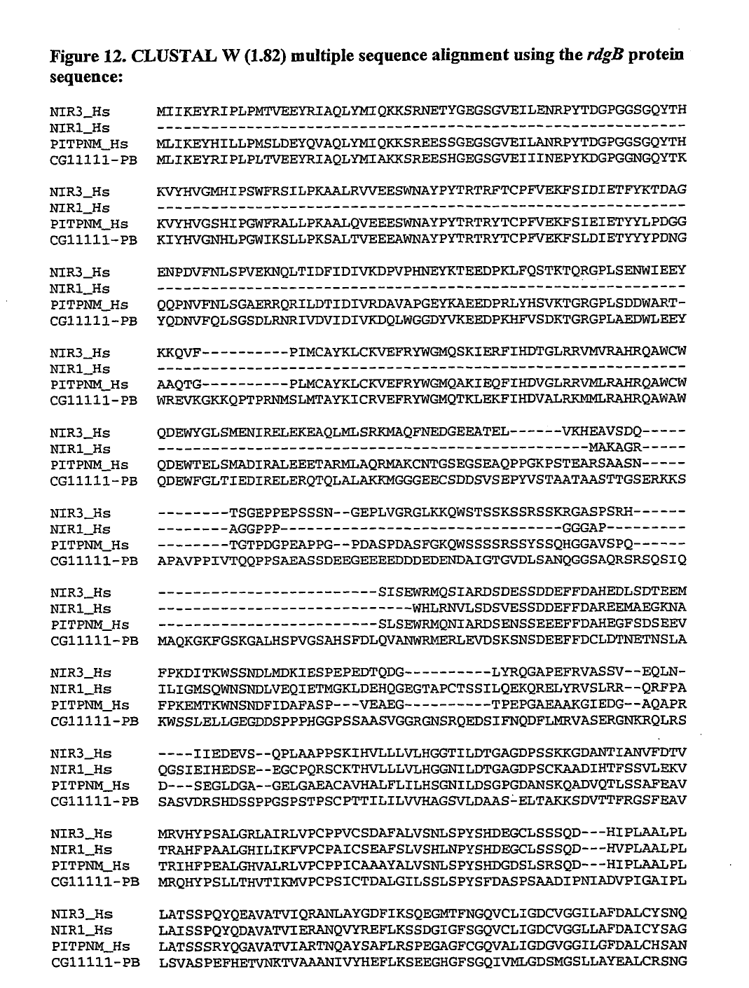

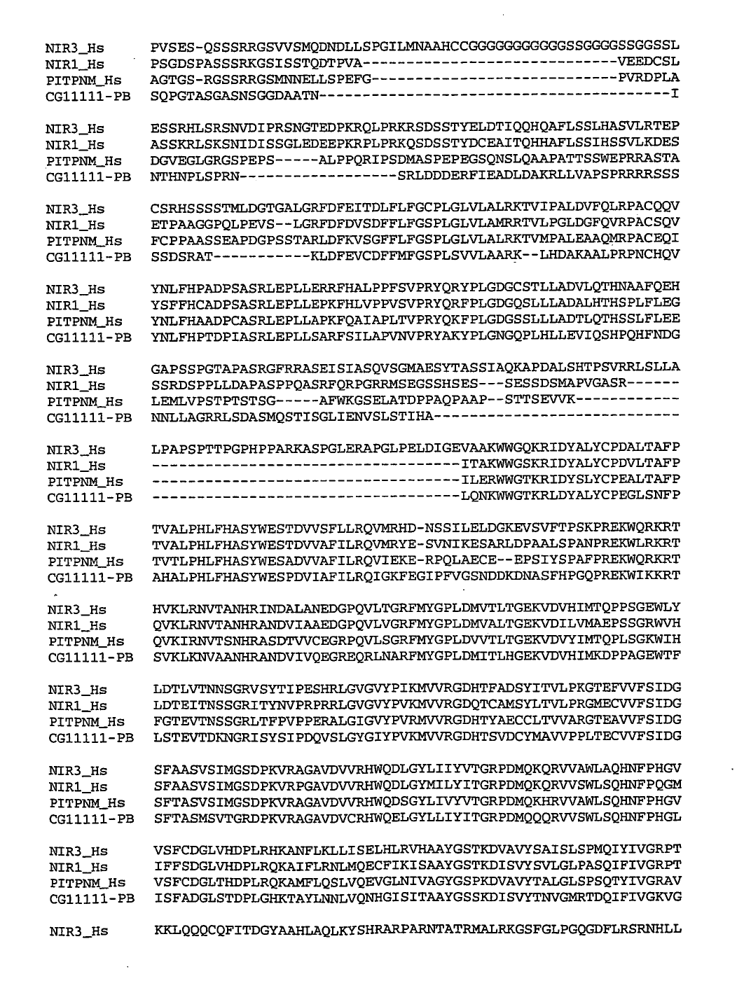

The Drosophila genes and proteins encoded thereby with functions in the regulation of triglyceride metabolism were further analysed using the BLAST algorithm searching in publicly available sequence databases and mammalian homologs were identified (see Table 1 and FIG. 12).

The term “polynucleotide comprising the nucleotide sequence as shown in GenBank Accession number” relates to the expressible gene of the nucleotide sequences deposited under the corresponding GenBank Accession number. The term “GenBank Accession number” relates to NCBI GenBank database entries (Ref.: Benson et al., Nucleic Acids Res. 28 (2000) 15-18). Sequences homologous to Drosphila guf, CG3811, CG30346 (CG8080), rdgB, Mekk1, Ady43A, CG14816, tws, or PP2A-B genes were identified using the publicly available program BLASTP 2.2.3 of the non-redundant protein data base of the National Center for Biotechnology Information (NOBI) (see, Altschul S. F. et al., (1997) Nucleic Acids Res. 25:3389-3402).

TABLE 1
Human homologs of the Drosophila (Dm) genes

| Dm gene | Homo sapiens homologous genes and proteins | | |
|---|---|---|---|
| Acc. No. | Accession Number | | |
| Name | cDNA | Protein | Name |
| CG16747 | NM_004152 | NP_004143 | ornithine decarboxylase antizyme 1 (OAZ1) |
| guf | MM_002537 | NP_002528 | ornithine decarboxylase antizyme 2 (OAZ2) |
| | NM_016178 | NP_057262 | ornithine decarboxylase antizyme 3 (OAZ3) |
| | AF293339 | AAG26885 | ornithine decarboxylase antizyme 4 (OAZ4) |
| CG3811 | NM_030958 | NP_112220 | solute carrier family 21 (organic anion |
| | | | transporter), member 15 (SLC21A15) |
| | | | organic anion transporter polypeptide- |
| | | | related protein 4 (OATPRP4) |
| CG30346 | NM_153013 | NP_694558 | hypothetical protein FLJ30596 (FLJ30596) |
| (CG8080) | | | |
| CG11111 | NM_004910 | NP_004901 | phosphatidylinositol transfer protein, |
| rdgB | | | membrane-associated 1 (PITPNM1) |
| | NM_020845 | NP_065896 | phosphatidylinositol transfer protein, |
| | | | membrane-associated 2 (PITPNM2) |
| | NM_031220 | NP_112497 | PITPNM family member 3 (PITPNM3) |
| CG7717 | AF042838 | AAC97073 | mitogen-activated protein kinase kinase |
| Mekk1 | | | kinase 1 (MAP3K1); |
| | | | MEK kinase 1 (MEKK1) |
| | NM_006609 | NP_006600 | mitogen-activated protein kinase kinase |
| | | | kinase 2 (MAP3K2) |
| | NM_002401 | NP_002392 | mitogen-activated protein kinase kinase |
| | | | kinase 3 (MAP3K3) |
| | NM_005922 | NP_005913 | mitogen-activated protein kinase kinase |
| | | | kinase 4 (MAP3K4) transcript variant 1 |
| | | | MAP/ERK kinase kinase 4 isoform a |
| | NM_006724 | NP_006715 | mitogen-activated protein kinase kinase |
| | | | kinase 4 (MAP3K4) transcript variant 2 |
| | | | MAP/ERK kinase kinase 4 isoform b |
| CG1851 | NM_001123 | NP_001114 | adenosine kinase (ADK), transcript variant |
| Ady43A | | | ADK-short; adenosine kinase isoform a |
| | NM_006721 | NP_006712 | adenosine kinase (ADK), transcript variant |
| | | | ADK-long; adenosine kinase isoform b |
| CG14816 | NM_138575 | NP_612642 | hypothetical protein MGC5352 (MGC5352) |
| EG: 63B12.4 | AK097688 | | FLJ40369 fis, clone TESTI2034847 |
| CG6235 | NM_002717 | NP_002708 | protein phosphatase 2 (formerly 2A), |
| tws | | | regulatory subunit B (PR 52), alpha isoform |
| | | | (PPP2R2A) |
| | NM_004576 | NP_004567 | protein phosphatase 2 (formerly 2A), |
| | | | regulatory subunit B (PR 52), beta isoform |
| | | | (PPP2R2B) |
| | NM_020416 | NP_065149 | protein phosphatase 2 (formerly 2A), |
| | | | regulatory subunit B (PR 52), gamma isoform |
| | | | (PPP2R2C), isoform a |
| | NM_181876 | NP_870991 | protein phosphatase 2 (formerly 2A), |
| | | | regulatory subunit B (PR 52), gamma isoform |
| | | | (PPP2R2C), isoform b |
| | AB040974 | BAA96065 | KIAA1541 protein |
| CG7913 | NM_002719 | NP_002710 | protein phosphatase 2, regulatory subunit B |
| PP2A-B′ | | | (B56), gamma isoform (PPP2R5C), isoform a |
| | NM_178586 | NP_848701 | protein phosphatase 2, regulatory subunit B |
| | | | (B56), gamma isoform (PPP2R5C), isoform b |
| | NM_178587 | NP_848702 | protein phosphatase 2, regulatory subunit B |
| | | | (B56), gamma isoform (PPP2R5C), isoform c |
| | NM_178588 | NP_848703 | protein phosphatase 2, regulatory subunit B |
| | | | (B56), gamma isoform (PPP2R5C), isoform d |
| | NM_006245 | NP_006236 | protein phosphatase 2, regulatory subunit B |
| | | | (B56), delta isoform (PPP2R5D), isoform 1 |
| | NM_180976 | NP_851307 | protein phosphatase 2, regulatory subunit B |
| | | | (B56), delta isoform (PPP2R5D), isoform 2 |
| | NM_180977 | NP_851308 | protein phosphatase 2, regulatory subunit B |
| | | | (B56), delta isoform (PPP2R5D), isoform 3 |
| CG7913 | NM_002719 | NP_002710 | protein phosphatase 2, regulatory |
| PP2A-B′ | | | subunit B (B56), gamma isoform |
| | | | (PPP2R5C), isoform a |
| | NM_178586 | NP_848701 | protein phosphatase 2, regulatory |
| | | | subunit B (B56), gamma isoform |
| | | | (PPP2R5C), isoform b |
| | NM_178587 | NP_848702 | protein phosphatase 2, regulatory |
| | | | subunit B (B56), gamma isoform |
| | | | (PPP2R5C), isoform c |
| | NM_178588 | NP_848703 | protein phosphatase 2, regulatory |
| | | | subunit B (B56), gamma isoform |
| | | | (PPP2R5C), isoform d |
| | NM_006245 | NP_006236 | protein phosphatase 2, regulatory |
| | | | subunit B (B56), delta isoform |
| | | | (PPP2R5D), isoform 1 |
| | NM_180976 | NP_851307 | protein phosphatase 2, regulatory |
| | | | subunit B (B56), delta isoform |
| | | | (PPP2R5D), isoform 2 |
| | NM_180977 | NP_851308 | protein phosphatase 2, regulatory |
| | | | subunit B (B56), delta isoform |
| | | | (PPP2R5D), isoform 3 |

guf, CG3811, CG30346 (CG8080), rdgB, Mekk1, Ady43A, CG14816, tws, or PP2A-B homologous proteins and nucleic acid molecules coding therefore are obtainable from insect or vertebrate species, e.g. mammals or birds. Particularly preferred are nucleic acids as described in Table 1.

The human hypothetical protein FLJ30596 is also referred to as Homo sapiens similar to Y17G7B.10a.p (LOC133686; GenBank Accession Number XM_{—}059667 for the cDNA, XP_{—}059667 for the protein). Human PITPNM1 is also referred to as human membrane associated phosphatidylinositol transfer protein (PITPNM) and human PYK2 N-terminal domain-interacting receptor 2 (NIR2), human PITPNM2 is also referred to as human PYK2 N-terminal domain-interacting receptor 3 (NIR3; GenBank Accession Number XM_{—}038799 for the cDNA, XP_{—}038799 for the protein), and human PITPNM3 is also referred to as human PYK2 N-terminal domain-interacting receptor 1 (NIR1), human PITPNM2 and PITPNM3 are also referred to in patent application WO98/16639. Human PPP2R2C is also referred to as GenBank Accession Number XM_{—}029744 for the cDNA, XP_{—}029744 for the protein. Human KIAA1541 protein is also referred to as GenBank Accession Number NM_{—}018461 for the cDNA, NP_{—}060931 for the protein.

The mouse homologous cDNAs encoding the polypeptides of the invention were identified as GenBank Accession Numbers NM_{—}008753 and XM_{—}125700 (for the mouse homolog of Oaz1), NM_{—}010952 (for the mouse homolog of Oaz2), NM_{—}016901 (for the mouse homolog of Oaz3), for the mouse homologs of guf, XM_{—}136452 for the mouse homolog of CG3811, XM_{—}127934 for the mouse homolog of CG30346, NM_{—}008851 (for the mouse homolog of PITPNM1), NM_{—}011256 (for the mouse homolog of PITPNM2), XM_{—}126447 (for the mouse homolog of PITPNM3) for the mouse homologs of rdgB, NM_{—}011948 for the mouse homolog of Mekk1, NM_{—}134079 and NM_{—}007411 for the mouse homologs of Ady43A, XM_{—}132261 for the mouse homolog of CG14816, XM_{—}127816 (for the mouse homolog of PPP2R2A), NM_{—}028392 (for the mouse homolog of PPP2R2B), XM_{—}144326 (for the mouse homolog of PPP2R2C), XM_{—}133882 (for the mouse homolog of PPP2R2D) for the mouse homologs of tws, and XM_{—}127113 (for the mouse homolog of PPP2R5C), NM_{—}009358, and XM_{—}128662 (for the mouse homolog of PPP2R5D) for the mouse homologs of PP2A-B′.

Sequences that were showing higher homologies to Drosophila rdgB (GadFly Accession Number CG11111) sequence than to any other protein family were collected in FASTA-format and aligned using the ClustaW alignment (see, Higgins D. G. et al., (1996) Methods Enzymol. 266:383-402; Thompson J. D. et al., (1994) Nucleic Acids Res. 22:4673-4680). All alignments were performed using standard parameters of the corresponding programs. According to the results of the multiple sequence alignment, PITPNM1 (referred to as ‘PITPNM_Hs’), PITPNM2 (referred to as ‘NIR3_Hs’), and PITPNM3 (referred to as ‘NIR1_Hs’) were identified as the direct human orthologs for Drosophila rdgB protein (herein referred to as ‘CG11111 _PB’). The alignment shown in FIG. 12 is comparing human PITPNMs with the rdgB sequence of Drosophila.

### Example 4

### dUCPy Modifier Screen

Expression of Drosophila uncoupling protein dUCPy in a non-vital organ like the eye (Gal4 under control of the eye-specific promoter of the ‘eyeless’ gene) results in flies with visibly damaged eyes. This easily visible eye phenotype is the basis of a genetic screen for gene products that can modify UCP activity.

Parts of the genomes of the strain with Gal4 expression in the eye and the strain carrying the pUAST-dUCPy construct were combined on one chromosome using genomic recombination. The resulting fly strain has eyes that are permanently damaged by dUCPy expression. Flies of this strain were crossed with flies of a large collection of mutagenized fly strains. In this mutant collection a special expression system (EP-element, Ref.: Rorth P., (1996) Proc Natl Acad Sci USA 93:12418-12422) is integrated randomly in different genomic loci. The yeast transcription factor Gal4 can bind to the EP-element and activate the transcription of endogenous genes close the integration site of the EP-element. The activation of the genes therefore occurs in the same cells (eye) that overexpress dUCPy. Since the mutant collection contains several thousand strains with different integration sites of the EP-element it is possible to test a large number of genes whether their expression interacts with dUCPy activity. In case a gene acts as an enhancer of UCP activity the eye defect will be worsened; a suppressor will ameliorate the defect.

Using this screen a gene with enhancing activity was discovered that was found to be the PP2A-B′ gene in Drosophila.

### Example 5

### Expression of the Polypeptides in Mammalian (mouse) Tissues

To analyse the expression of the polypeptides disclosed in this invention in mammalian tissues, several mouse strains (preferrably mice strains C57BI/6J, C57BI/6 ob/ob and C57BI/KS db/db which are standard model systems in obesity and diabetes research) were purchased from Harlan Winkelmann (33178 Borchen, Germany) and maintained under constant temperature (preferrably 22° C. ), 40 per cent humidity and a light/dark cycle of preferrably 14/10 hours. The mice were fed a standard chow (for example, from ssniff Spezialitäten GmbH, order number ssniff M-Z V1126-000). For the fasting experiment (“fasted wild type mice”), wild type mice were starved for 48 h without food, but only water supplied ad libitum (see, for example, Schnetzler B. et al., 1993, J Clin Invest 92: 272-280, Mizuno T. M. et al., 1996, Proc Natl Acad Sci USA 93:3434-3438). In a further experiment wild-type (wt) mice were fed a control diet (preferably Altromin C1057 mod control, 4.5% crude fat) or high fat diet (preferably Altromin C1057mod. high fat, 23.5% crude fat). Animals were sacrificed at an age of 6 to 8 weeks. The animal tissues were isolated according to standard procedures known to those skilled in the art, snap frozen in liquid nitrogen and stored at −80° C. until needed.

For analyzing the role of the proteins disclosed in this invention in the in vitro differentiation of mammalian cell culture cells for the conversion of pre-adipocytes to adipocytes, mammalian fibroblast (3T3-L1) cells (e.g., Green H. and Kehinde O., 1974 Cell 1:113-116) were obtained from the American Tissue Culture Collection (ATCC, Hanassas, Va., USA; ATCC-CL 173). 3T3-L1 cells were maintained as fibroblasts and differentiated into adipocytes as described in the prior art (e.g., Qiu Z. et al., 2001, J. Biol. Chem. 276: 11988-11995; Slieker L. J. et al., 1998, BBRC 251: 225-229). In brief, cells were plated in DMEM/10% FCS (Invitrogen, Karlsruhe, Germany) at 50,000 cells/well in duplicates in 6-well plastic dishes and cultured in a humidified atmosphere of 5% CO₂ at 37° C. At confluence (defined as day 0: d0) cells were transferred to serum-free (SF) medium, containing DMEM/HamF12 (3:1; Invitrogen), fetuin (300 μl; Sigma, Munich, Germany), transferrin (2 μg/ml; Sigma), pantothenate (17 μM; Sigma), biotin (1μM; Sigma), and EGF (0.8 μM; Hoffmann-La Roche, Basel, Switzerland). Differentiation was induced by adding Dexamethasone (DEX; 1 μM; Sigma), 3-methyl-isobutyl-1-methylxanthine (MIX; 0.5 mM; Sigma), and bovine insulin (5 μg/ml; Invitrogen). Four days after confluence (d4), cells were kept in SF medium, containing bovine insulin (5 μg/ml) until differentiation was completed. At various time points of the differentiation procedure, beginning with day 0 (day of confluence) and day 2 (hormone addition; for example, dexamethasone and 3-isobutyl-1-methylxanthine), up to 10 days of differentiation, suitable aliquots of cells were taken every two days.

RNA was isolated from tissues and cells using Trizol Reagent (for example, from Invitrogen, Karlsruhe, Germany) and further purified with the RNeasy Kit (for example, from Qiagen, Germany) in combination with an DNase-treatment according to the instructions of the manufacturers and as known to those skilled in the art. Total RNA was reverse transcribed (preferrably using Superscript II RNaseH Reverse Transcriptase, from Invitrogen, Karlsruhe, Germany) and subjected to Taqman analysis preferrably using the Taqman

2×PCR Master Mix (from Applied Biosystems, Weiterstadt,. Germany; the Mix contains according to the Manufacturer for example AmpliTaq Gold DNA Polymerase, AmpErase UNG, dNTPs with dUTP, passive reference Rox and optimized buffer components) on a GeneAmp 5700 Sequence Detection System (from Applied Biosystems, Weiterstadt, Germany).

Taqman analysis was performed preferably using the following primer/probe pairs:

For the amplification of mouse ornithine decarboxylase antizyme 1 (Oaz1) sequence (GenBank Accession Number NM_{—}008753):

Mouse Oaz1forward primer (SEQ ID NO: 1): 5′-CAG CAG CGA GAG TTC TAG GGT T-3′; mouse Oaz1 reverse primer (SEQ ID NO: 2): 5′-AGG AGO ACC ACC GAG GO-3′; mouse Oaz1Taqman probe (SEQ ID NO: 3): (5/6-FAM)-CCC TTA ATT GCT GTA GTA ACC TGG GTC CGG-(5/6-TAMRA).

For the amplification of mouse ornithine decarboxylase antizyme 2 (Oaz2) sequence (GenBank Accession Number NM_{—}010952):

Mouse Oaz2 forward primer (SEQ ID NO: 4): 5′-TAT TTT GCC GTT GAG TAA GTG TCC-3′; mouse Oaz2 reverse primer (SEQ ID NO: 5): 5′-CAC CAC AGA GGC CCT GGA-3′; mouse Oaz2 Taqman probe (SEQ ID NO: 6):(5/6-FAM)-AGC TCC AGT GCT GCA GGC ACA TTG -(5/6-TAMRA).

For the amplification of mouse ornithine decarboxylase antizyme 3 (Oaz3) sequence (GenBank Accession Number NM_{—}016901):

Mouse Oaz3 forward primer (SEQ ID NO: 7): 5′ -CAA ATC GAT CGG AAG GAC AGA-3′; mouse Oaz3 reverse primer (SEQ ID NO: 8): 5′-CCA CCT CGA AGC CCA TGT -3′; mouse Oaz3 Taqman probe (SEQ ID NO: 9): (5/6-FAM)-TGC CCT GCT GCG AGC CTT TAG C -(5/6-TAMRA).

For the amplification of mouse protein similar to organic anion transporter polypeptide-related protein 4 (LOC240726) sequence (GenBank Accession Number XM_{—}136452):

Mouse LOC240726 forward primer (SEQ ID NO: 10): 5′-GCC TTG CGG TGG GCA-3′; mouse LOC240726 reverse primer (SEQ ID NO: 11): 5′-AGC GCA ACT GTC CGA ACA A-3′; mouse LOC240726 Taqman probe (SEQ ID NO: 12): (5/6-FAM)-ACA CCC CTG GAG CCA CAA AGT GGG-(5/6-TAMRA).

For the amplification of mouse RIKEN cDNA 1110020G09 gene (1110020G09Rik) sequence (GenBank Accession Number XM_{—}127934):

Mouse 1110020G09Rik forward primer (SEQ ID NO: 13): 5′-TGC CTG TGC GAT ACA CGC-3′; mouse 1110020G09Rik reverse primer (SEQ ID NO: 14): 5′-AAC CAC CTG AAC TCA CCA CGA-3′; mouse 1110020G09Rik Taqman probe (SEQ ID NO: 15): (5/6-FAM)-TTC CTT CCC TGA AGC CTT ACG GAG GTT-(5/6-TAMRA).

For the amplification of mouse phosphatidylinositol membrane-associated (Pitpnm) sequence (GenBank Accession Number NM_{—}008851):

Mouse Pitpnm forward primer (SEQ ID NO: 16): 5′-CAC ACA GGT CAA GAT CCG GA-3′; mouse Pitpnm reverse primer (SEQ ID NO: 17): 5′-AGC GCC CAT TCA GCA CC-3′; mouse Pitpnm Taqman probe (SEQ ID NO: 18): (5/6-FAM)-CAC TTC CAA CCA CCG AGC GAG TGA C-(5/6-TAMRA).

For the amplification of mouse retinal degeneration B2 homolog (Drosophila) (Rdgb2) sequence (GenBank Accession Number NM_{—}01 1256):

Mouse Rdgb2 forward primer (SEQ ID NO: 19): 5′-TGA GCC GCA GCA ACA TTG -3′; mouse Rdgb2 reverse primer (SEQ ID NO: 20): 5′-CTG GAT AGG AAG GCC TGG TG-3′; mouse Rdgb2 Taqman probe (SEQ ID NO: 21): (5/6-FAM)-CCG AAG CAA CGG CAC TGA GGA CTC-(5/6-TAMRA).

For the amplification of mouse similar to PYK2 N-terminal domain-interacting receptor 1 (LOC216884) sequence (GenBank Accession Number XM_{—}126447):

Mouse LOC216884 forward primer (SEQ ID NO: 22): 5′-AAG TGG CTG CGT AAG CGA A-3′; mouse LOC216884 reverse primer (SEQ ID NO: 23): 5′-ACA TCA TTG GCA CGG TGG T-3′; mouse LOC216884 Taqman probe (SEQ ID NO: 24): (5/6-FAM)-CCA GGT CAA GTT GCG GAA CGT TAC TGC-(5/6-TAMRA).

For the amplification of mouse mitogen activated protein kinase kinase kinase 4 (Map3k4) sequence (GenBank Accession Number NM_{—}011948):

Mouse Map3k4 forward primer (SEQ ID NO: 25): 5′-CAG CCC ATC ATC GCC AA-3′; mouse Map3k4 reverse primer (SEQ ID NO: 26): 5′-GCA TCG CTG ATT CTG TTG CA-3′; mouse Map3k4 Taqman probe (SEQ ID NO: 27): (5/6-FAM)-TTG CAG CAG CTC AAG AAC GAT GCA CT-(5/6-TAMRA).

For the amplification of mouse adenosine kinase (Adk) sequence (GenBank Accession Number NM_{—}134079):

Mouse Adk forward primer (SEQ ID NO: 28): 5′-AGC CCA AAA AGC TCA AGG TG-3′; mouse Adk reverse primer (SEQ ID NO: 29): 5′-TTC CCC ATT CCA AAT AGC ACA-3′; mouse Adk Taqman probe (SEQ ID NO: 30): (5/6-FAM)-AGC GCC ACA AGC GCT GAG TGA A-(5/6-TAMRA).

For the amplification of mouse RIKEN cDNA 2610528A17 gene (2610528A17Rik) sequence (GenBank Accession Number XM_{—}132261):

Mouse 2610528A17Rik forward primer (SEQ ID NO: 31): 5′-TGT CCC TCA ACA ACG GCA G-3′; mouse 2610528A17Rik reverse primer (SEQ ID NO: 32): 5′-TCC TGA GTG CCA CAC GAC C -3′; mouse 2610528A17Rik Taqman probe (SEQ ID NO: 33): (5/6-FAM)-CAC CCA CCT GGT GAT TCG ACC CA-(5/6-TAMRA).

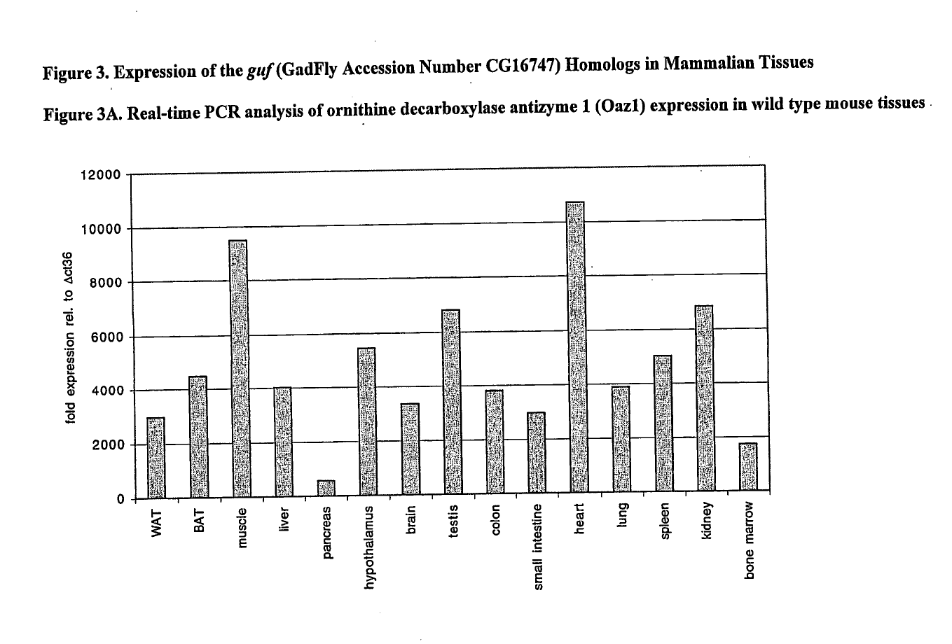

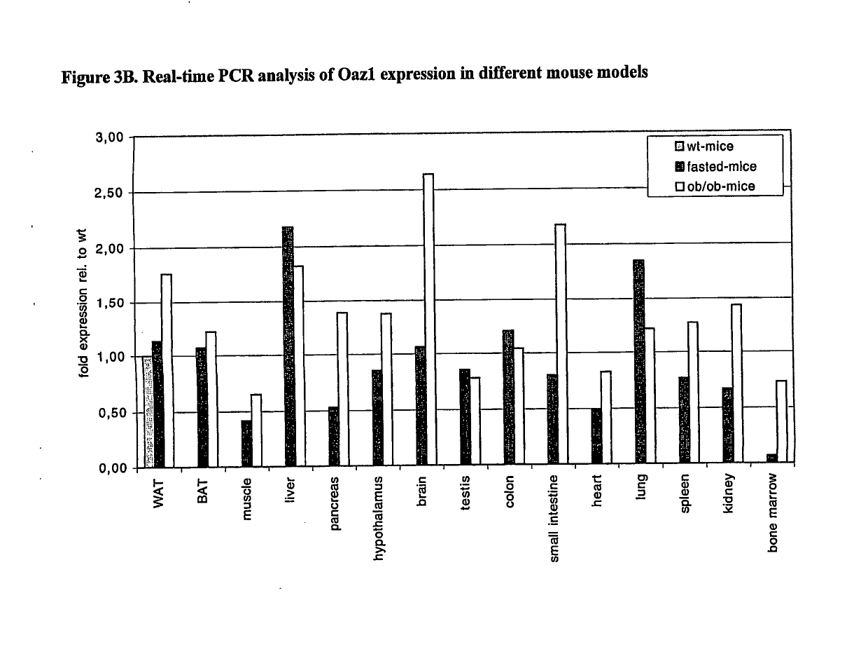

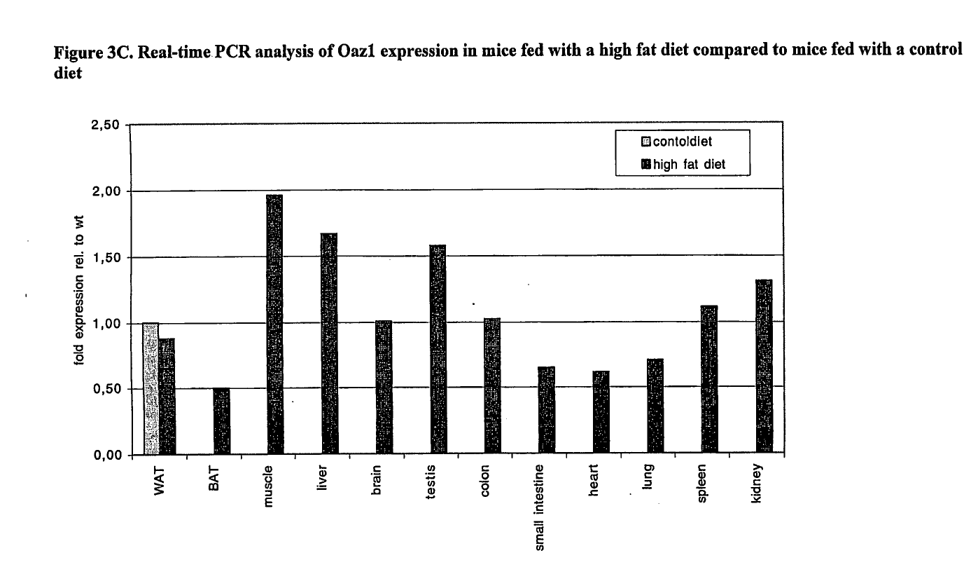

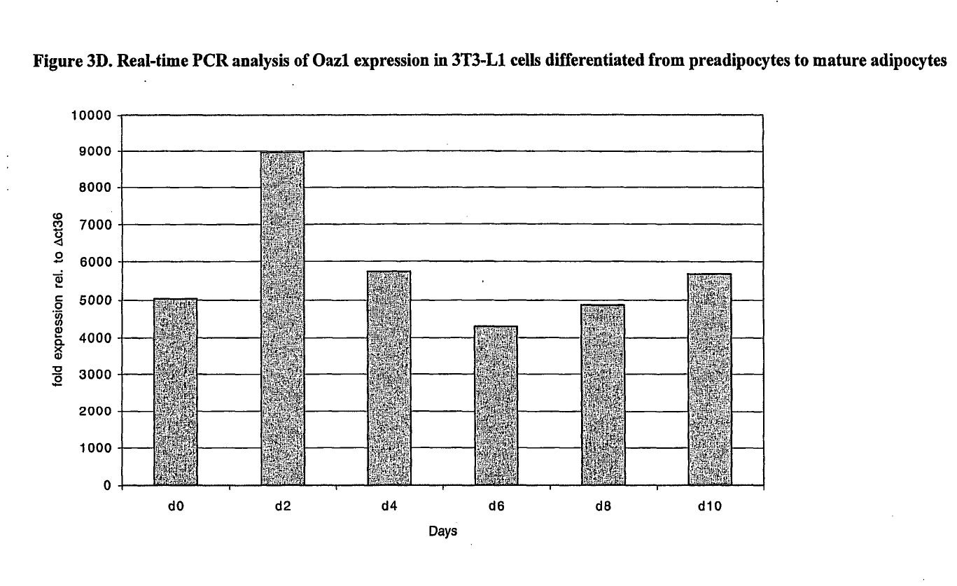

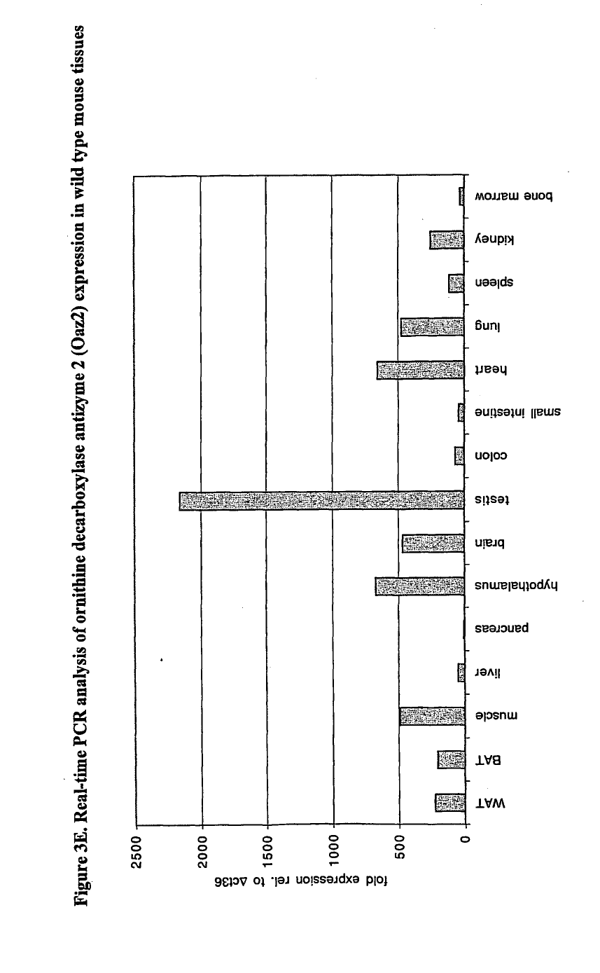

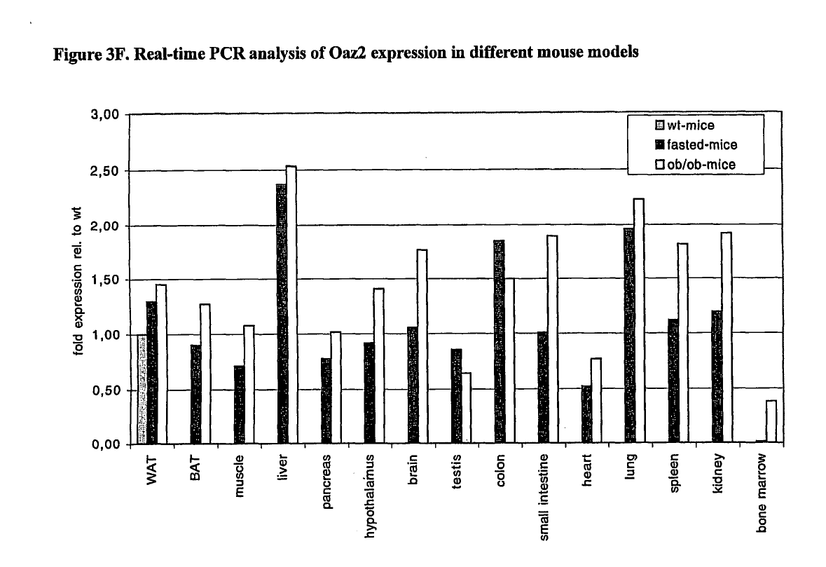

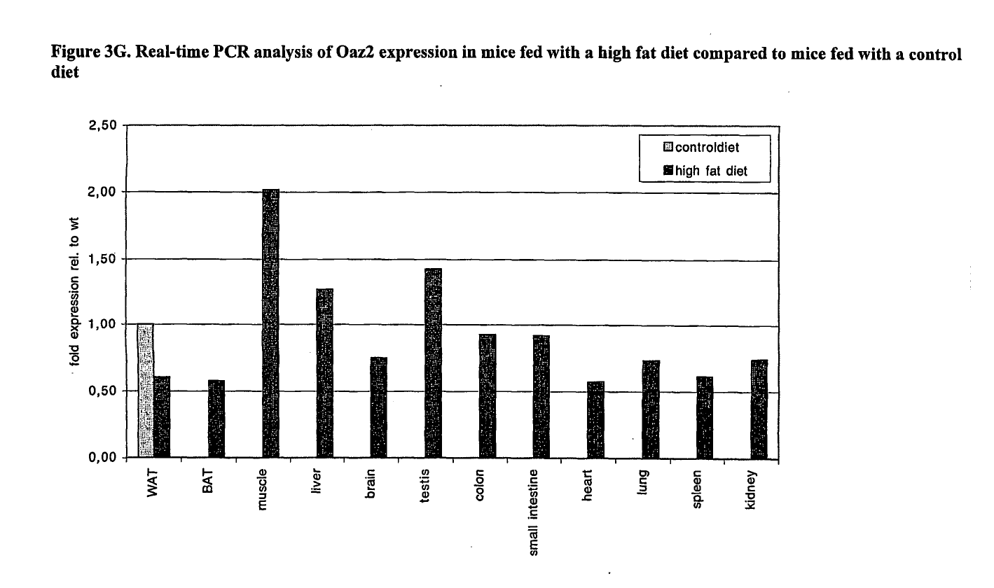

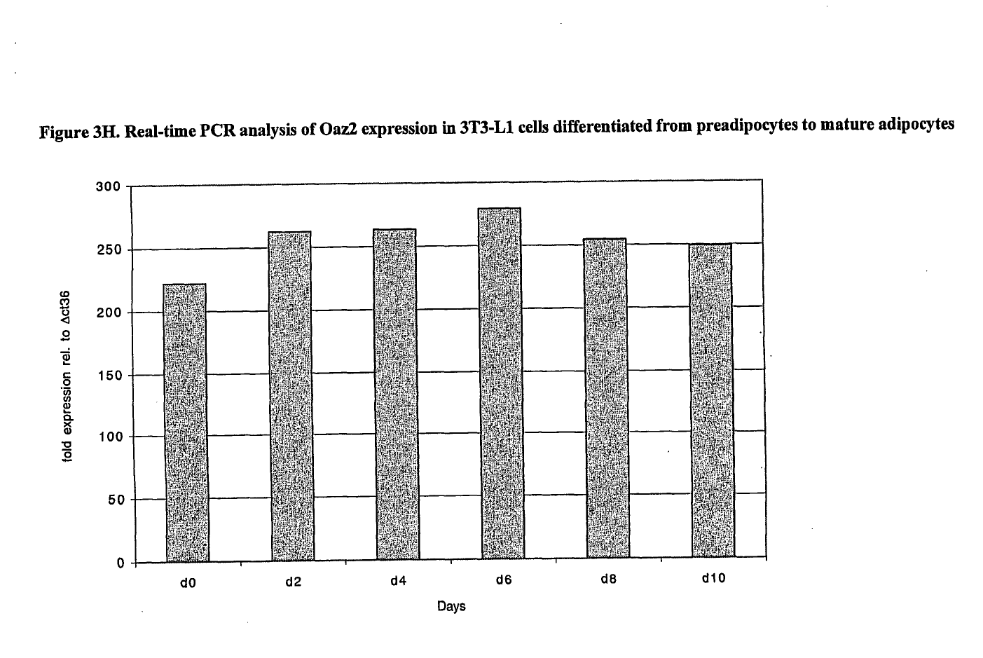

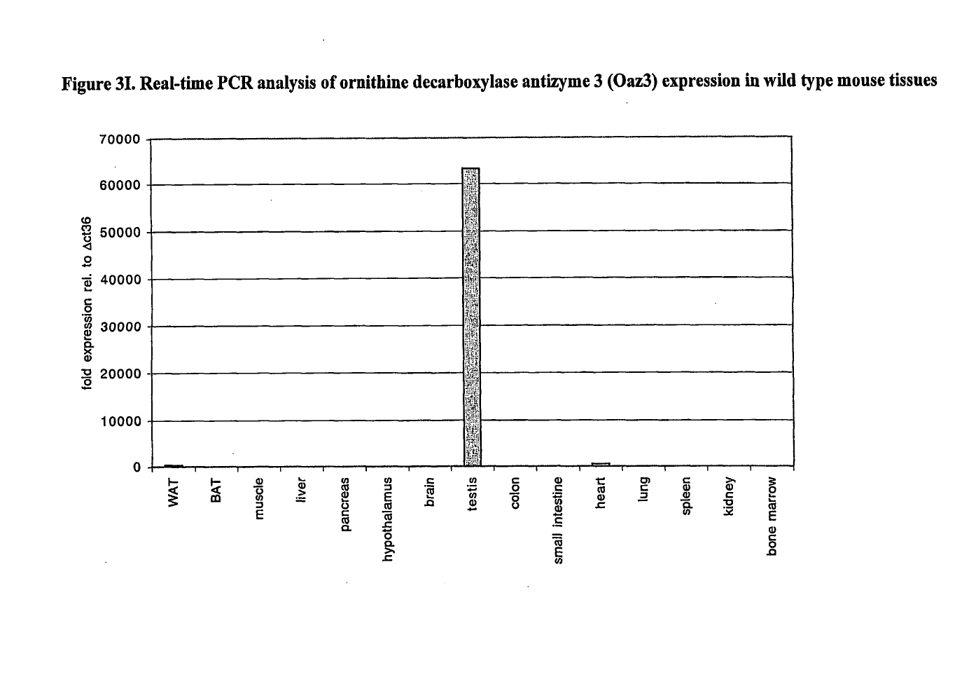

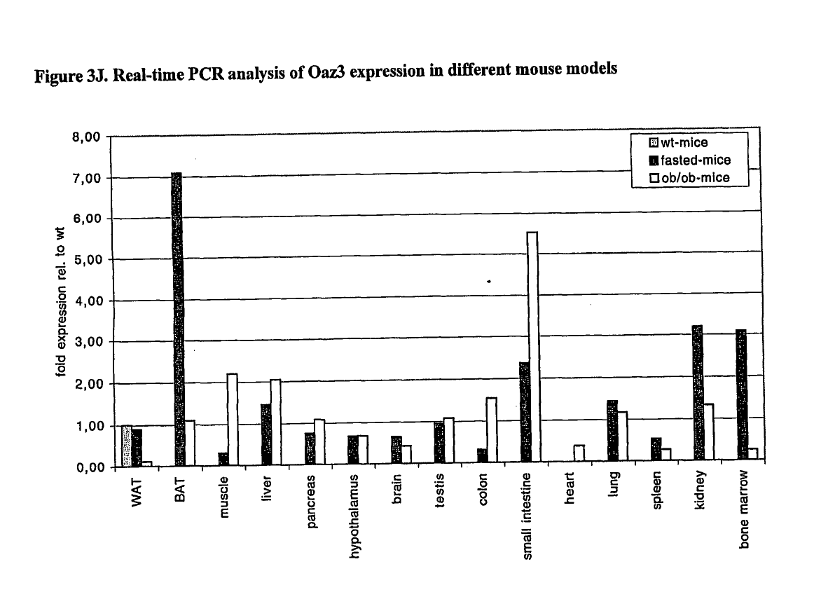

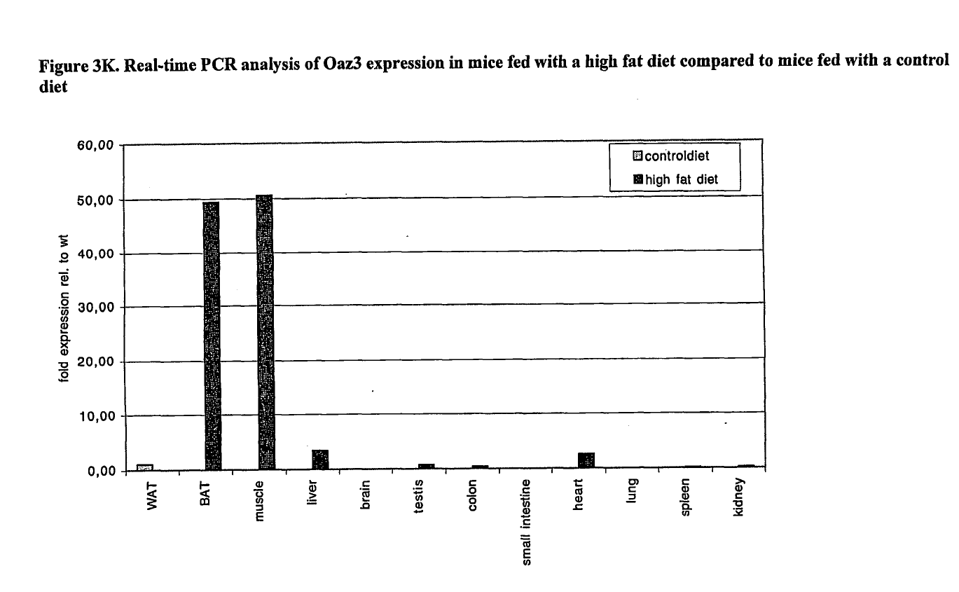

In the figures the relative RNA-expression is shown on the Y-axis. In FIGS. 3A-C, 3-E-G, 3l-K, 6A-C, 9A-B, 13A-C, 13E-F, 13H-J, 16A-B, 19A-C, and 22A-C, the tissues tested are given on the X-axis. “WAT” refers to white adipose tissue, “BAT” refers to brown adipose tissue. In FIG. 3D, 3H, 9C, 13D, 13G, 16C, 19D, and 22D, the X-axis represents the time axis. “d0” refers to day 0 (start of the experiment), “d2”-“d10” or “d12” refers to day 2-day 10 or day 12 of adipocyte differentiation.

The function of the proteins of the invention in metabolism was further validated by analyzing the expression of the transcripts in different tissues and by analyzing the role in adipocyte differentiation.

Mouse models of insulin resistance and/or diabetes were used, such as mice carrying gene knockouts in the leptin pathway (for example, ob/ob (leptin) or db/db (leptin receptor/ligand) mice) to study the expression of the proteins of the invention. Such mice develop typical symptoms of diabetes, show hepatic lipid accumulation and frequently have increased plasma lipid levels (see Bruning J. C. et al, (1998) Mol. Cell. 2: 559-569).

Expression of the mRNAs encoding the proteins of the invention was also examined in susceptible wild type mice (for example, C57BI/6) that show symptoms of diabetes, lipid accumulation, and high plasma lipid levels, if fed a high fat diet.

Expression profiling studies confirm the particular relevance of the proteins of the present invention as regulators of energy metabolism in mammals.

Taqman analysis revealed that ornithine decarboxylase antizyme 1 (Oaz1) is expressed in several mammalian tissues, showing highest level of expression in heart and muscle and higher levels in further tissues, e.g. white adipose tissue (WAT), brown adipose tissue (BAT), liver, hypothalamus, brain, testis, colon, small intestine, lung, spleen, kidney, and bone marrow. Furthermore, Oaz1 is expressed on a lower but still robust levels in the pancreas of wild type mice as depicted in FIG. 3A. We found, for example, that the expression of Oaz1 is down-regulated in muscle, heart, and bone marrow of fasted mice, and upregulated in the liver of fasted mice compared to wild type mice. We also found that the expression of Oaz1 is up-regulated in the brain and small intestine of ob/ob mice compared to wildtype mice (see FIG. 3B). In wild type mice fed a high fat diet, the expression of Oaz1 is down-regulated in BAT, as depicted in FIG. 3C. We further show in this invention (see FIG. 3D) that the Oaz1 mRNA is expressed during the differentiation into mature adipocyctes. The ubiqitous and robust expresssion levels of Oaz1 shows that it plays an essential role in cellular metabolism. The regulation of gene expression mainly in brain and small intestine shows that Oaz1 plays a role in the regulation of energy homeostasis in mammals.

Taqman analysis revealed that ornithine decarboxylase antzyme 2 (Oaz2) is expressed in several mammalian tissues, showing highest level of expression in testis and higher levels in further tissues, e.g. white adipose tissue (WAT), brown adipose tissue (BAT), muscle, hypothalamus, brain, heart, lung, spleen, and kidney. Furthermore, Oaz2 is expressed on a lower but still robust levels in the liver, colon, small intestine, and bone marrow of wild type mice as depicted in FIG. 3E. We found, for example, that the expression of Oaz2 is down-regulated inbone marrow of fasted mice, and up-regulated in the liver of fasted mice compared to wild type mice. We also found that the expression of Oaz2 is up-regulated in the liver of ob/ob mice, and down-regulated in the bone marrow of ob/ob mice compared to wildtype mice (see FIG. 3F). In wild type mice fed a high fat diet, the expression of Oaz2 is up-regulated in muscle, as depicted in FIG. 3G. We show in this invention (see FIG. 3H) that the Oaz2 mRNA is expressed during the differentiation into mature adipocyctes. The expression of Oaz2 in metabolic active tissues of wild type mice, as well as the regulation of Oaz2 in the liver and muscle in different animal models used to study metabolic disorders, shows that this gene plays a central role in energy homeostasis.

Taqman analysis revealed that ornithine decarboxylase antizyme 3 (Oaz3) is highly expressed in testis. Furthermore Oaz3 is expressed on a lower but very robust levels in WAT and heart of wild type mice as depicted in FIG. 31. We found, for example, that the expression of Oaz3 is significantly down-regulated in the heart of fasted mice. We also found that the expression of Oaz3 is significantly down-regulated in the WAT and heart of ob/ob mice compared to wildtype mice (see FIG. 3J). In wild type mice fed a high fat diet, the expression of Oaz3 is significantly down-regulated in WAT and up-regulated in heart, as depicted in FIG. 3K. The expression of Oaz3 in metabolic active tissues of wild type mice, as well as the regulation of Oaz3 specifically in WAT and heart in different animal models used to study metabolic disorders, suggests that this gene plays a central role in energy homeostasis. Oaz3 is not expressed during 3T3-L1 adipocyte differentiation (data not shown). This suggests that Oaz3 does not play a role in adipogenesis. However as Oaz3 is expressed and regulated by metabolic stressors e.g., fasting, high fat diet in our animal models, it is assumed that the gene becomes induced by specific signals in response to metabolic changes.

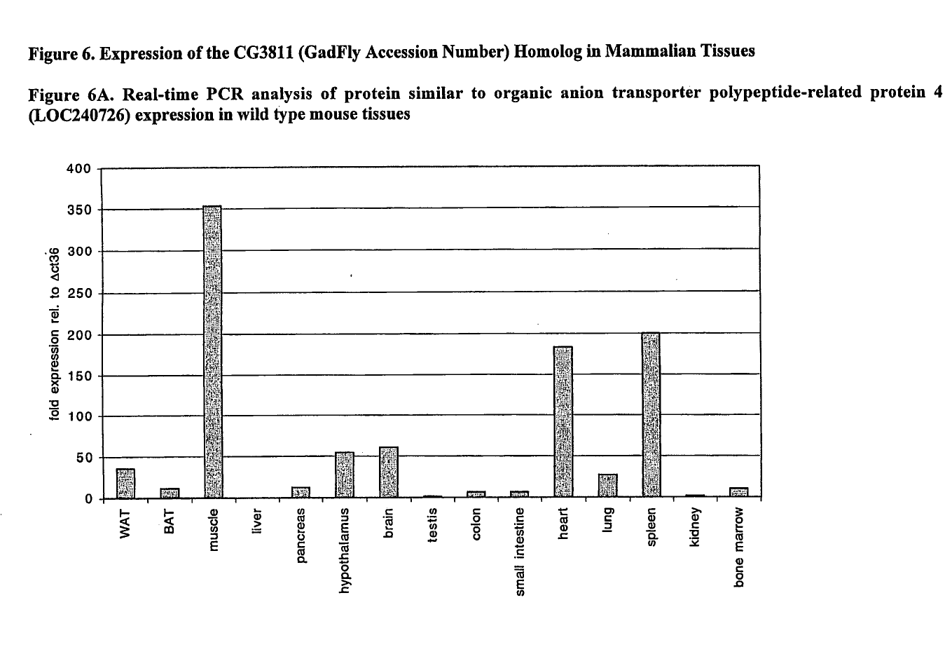

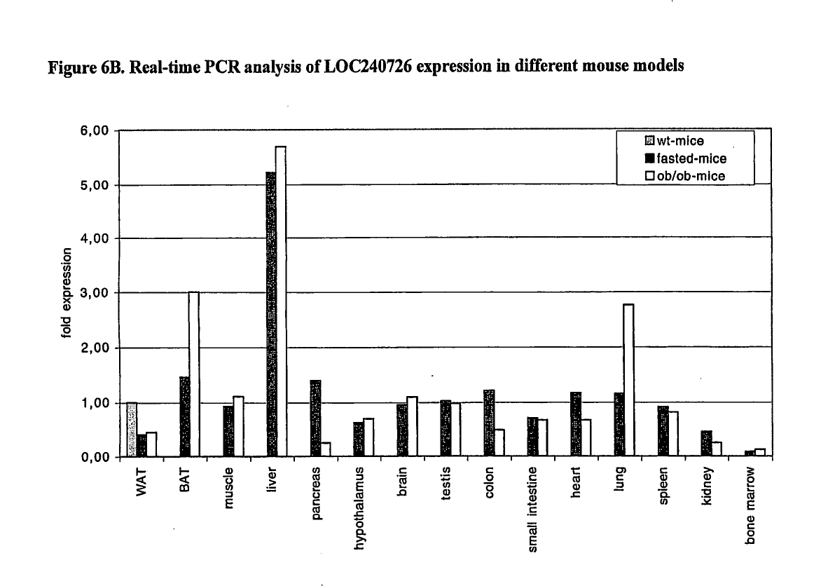

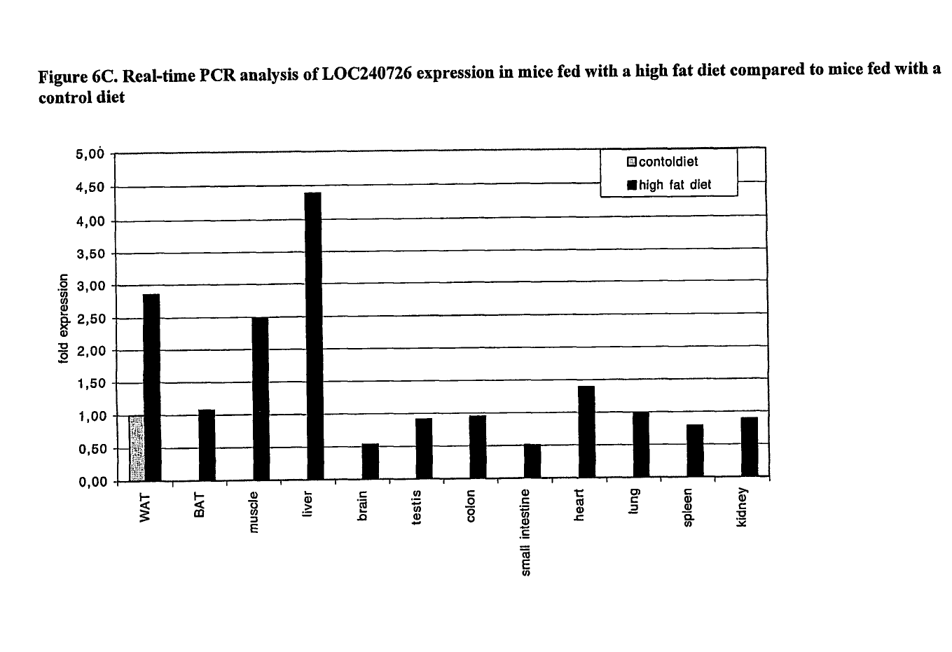

Taqman analysis revealed highest level of expression of the protein similar to organic anion transporter polypeptide-related protein 4 (LOC240726) in the muscle of wild type mice, and high levels of expression in heart and spleen. Furthermore LOC240726 is expressed on lower but still robust levels in WAT, hypothalamus, brain, and lung of wild type mice as depicted in FIG. 6A. We found, for example, that the expression of LOC240726 is significantly down-regulated in the WAT of fasted mice compared to wild type mice. We also found that the expression of LOC240726 is significantly down-regulated in the WAT and up-regulated in the lung of ob/ob mice compared to wild type mice (see FIG. 6B). In wild type mice fed a high fat diet, the expression of LOC240726 is significantly up-regulated in WAT and muscle as depicted in FIG. 6C. The regulated expression of LOC240726 in different animal models used to study metabolic disorders suggests that this gene plays a central role in energy homeostasis.

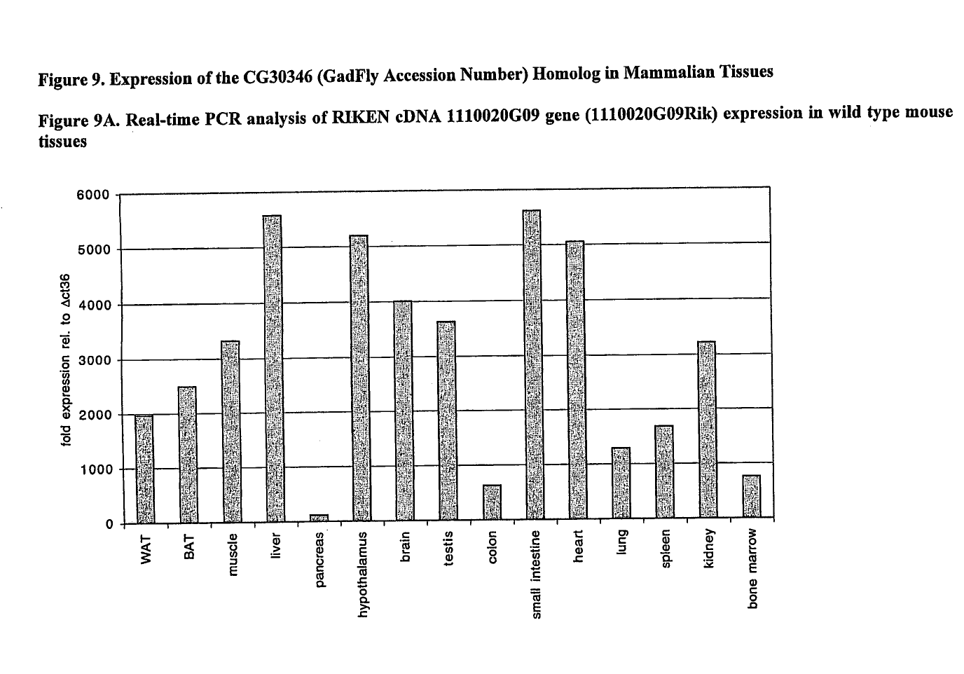

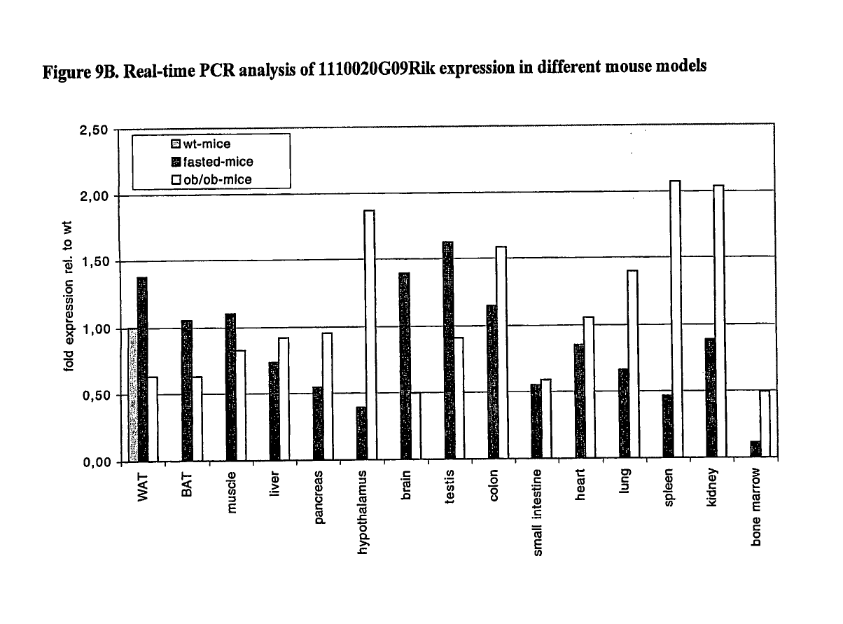

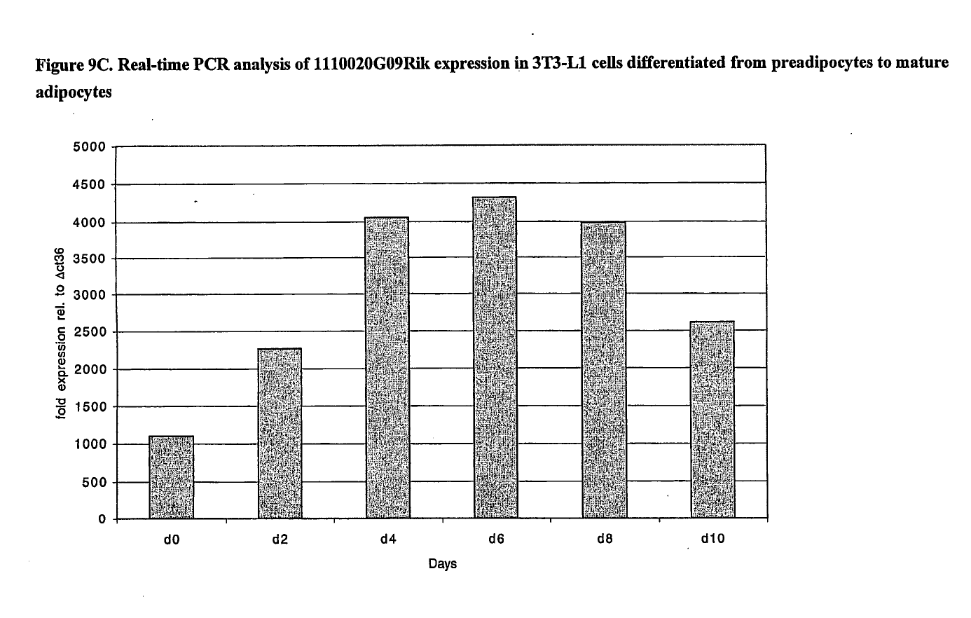

Taqman analysis revealed that RIKEN cDNA 1110020G09 gene (1110020G09Rik) is expressed in several mammalian tissues, showing highest level of expression in liver, hypothalamus, small intestine, and heart and higher levels in further tissues, e.g. WAT, BAT, muscle, brain, testis, lung, spleen, and kidney. Furthermore 1110020G09Rik is expressed on lower but still robust levels in pancreas, colon, and bone marrow of wild type mice as depicted in FIG. 9A. We found, for example, that the expression of 1110020G09Rik is down-regulated inthe brain and bone marrow, and up-regulated in the spleen and kidney of genetically induced obese mice (ob/ob) compared to wild type mice. Furthermore 1110020G09Rik is down-regulated in the hypothalamus, spleen, and bone marrow of fasted mice compared to wild type mice (see FIG. 9B). We show in this invention (see FIG. 9C) that the 1110020G09Rik mRNA is expressed and up-regulated during the differentiation into mature adipocyctes. Therefore, the 1110020G09Rik protein might play an essential role in adipogenesis. The expression of 1110020G09Rik in metabolic active tissues and the regulated expression in tissues of different animal models used to study metabolic disorders, together with the regulation during the differentiation from preadipocytes to mature adipocytes, shows that this gene plays a central role in energy homeostasis.

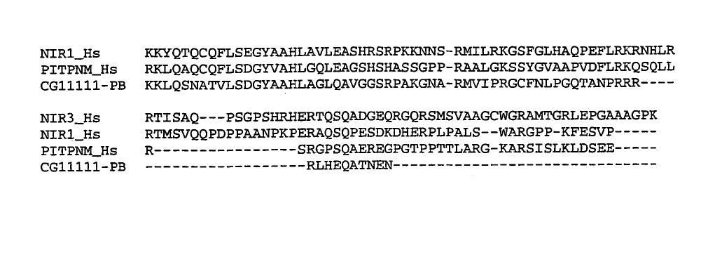

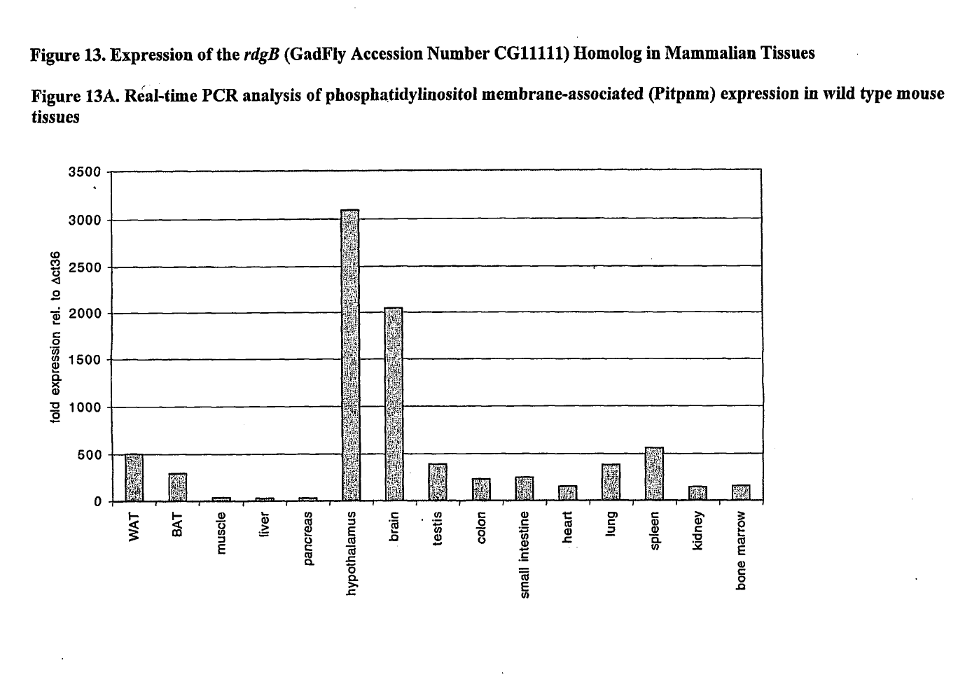

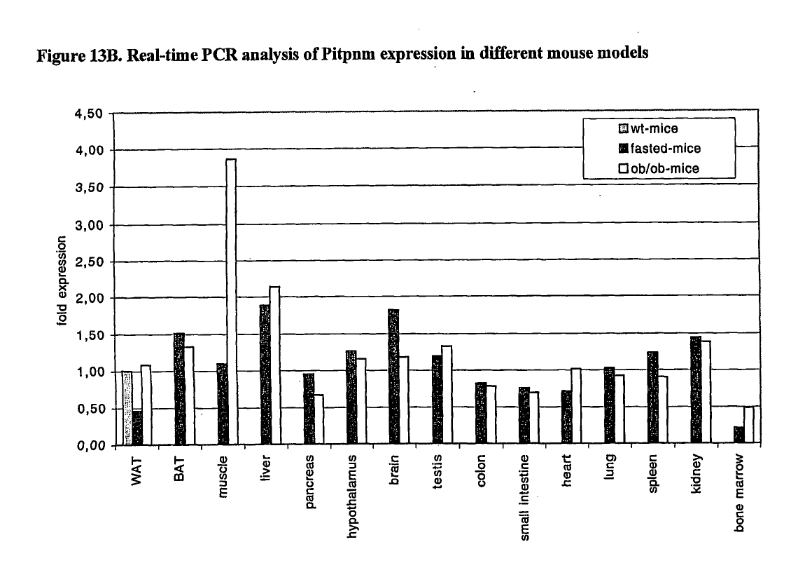

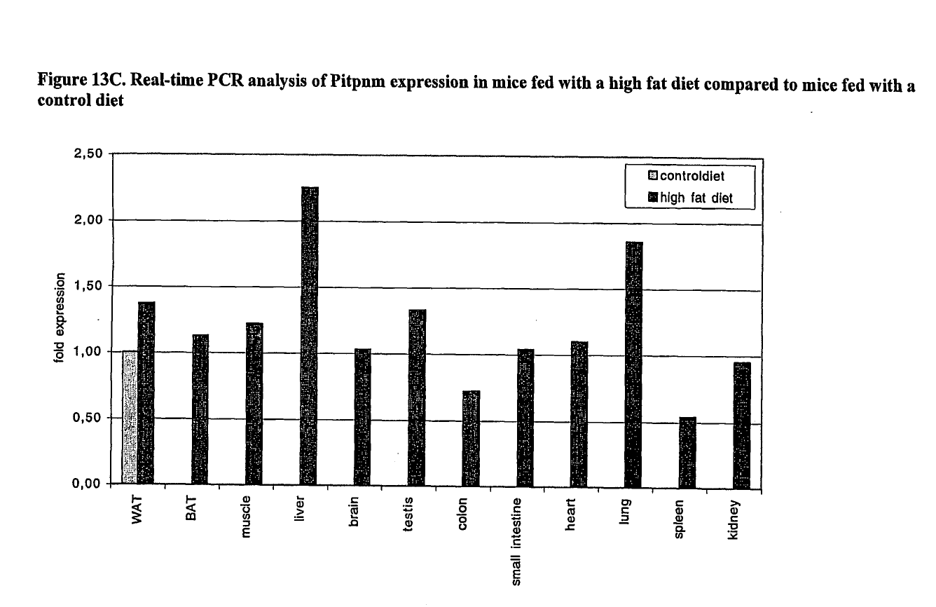

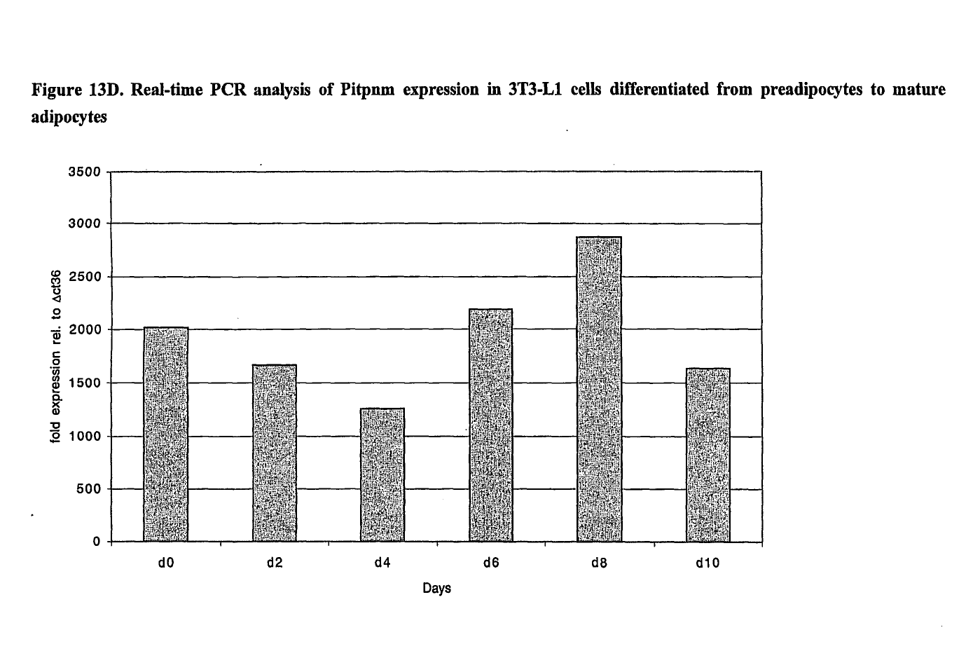

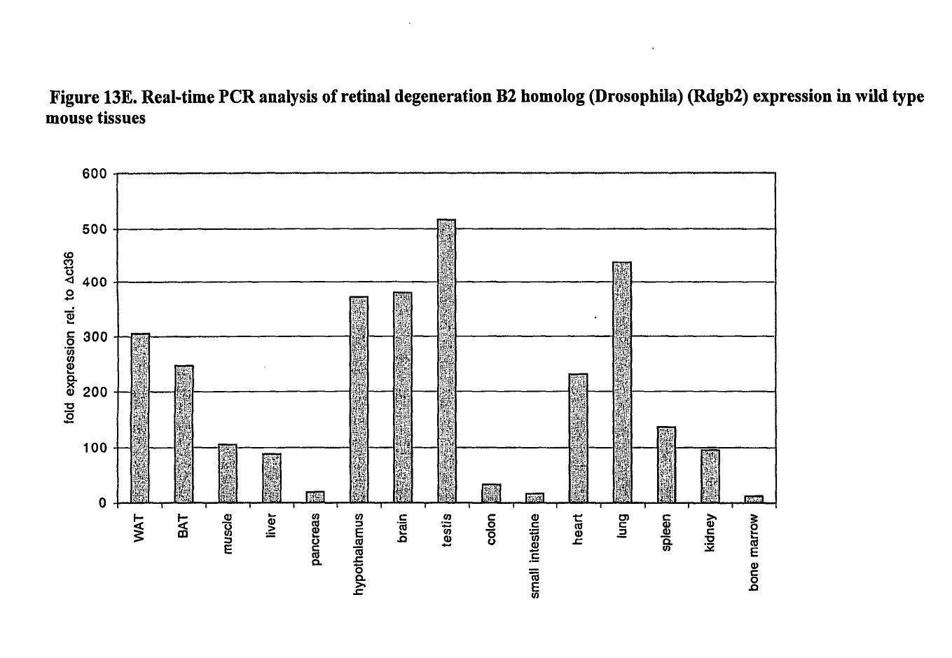

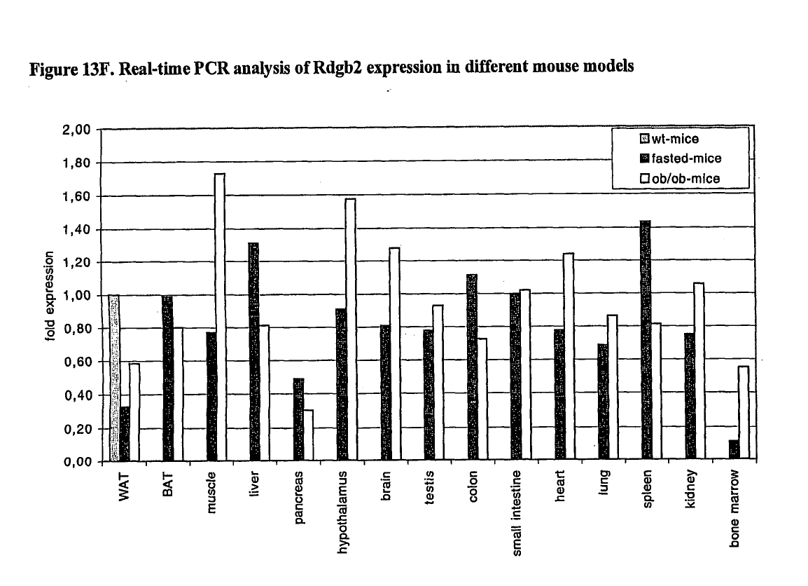

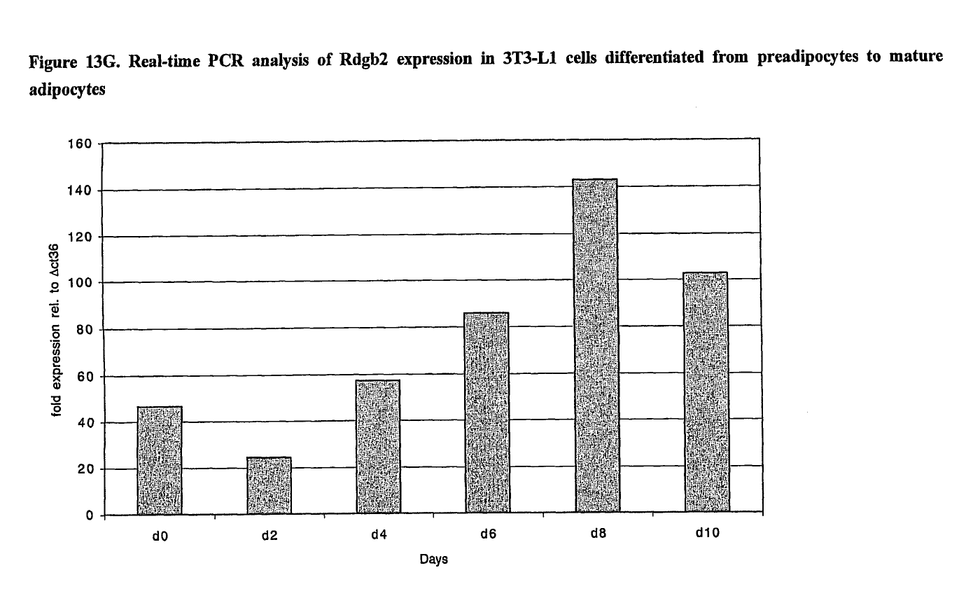

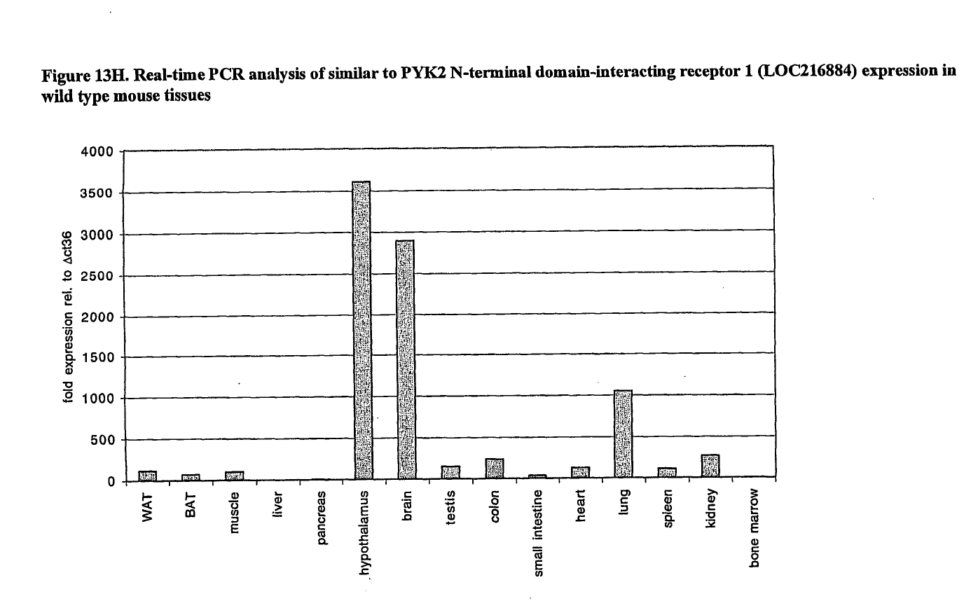

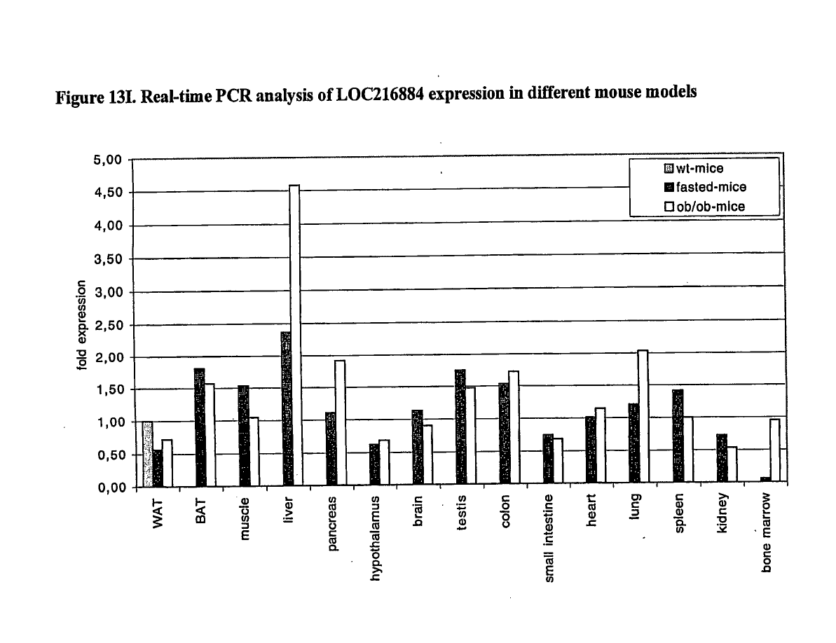

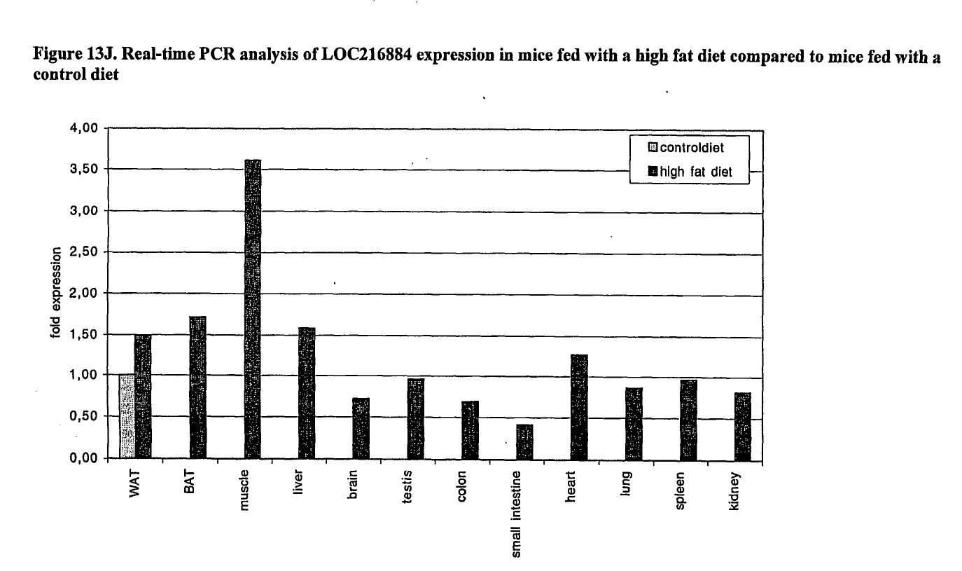

Taqman analysis revealed that phosphatidylinosiol membrane-associated (Pitpnm) is expressed in several mammalian tissues, showing highest levels of expression in hypothalamus and brain and higher levels in further tissues, e.g. WAT, BAT, testis, colon, small intestine, heart, lung, spleen, kidney, and bone marrow. Furthermore Pitpnm is expressed on a lower but still robust levels in muscle, liver and pancreas of wild type mice as depicted in FIG. 13A. We found, for example, that the expression of Pitpnm is down-regulated inWAT and bone marrow of fasted mice compared to wild type mice. We also found that the expression of Pitpnm is up-regulated in muscle and liver, and down-regulated in the bone marrow of ob/ob mice compared to wildtype mice (see FIG. 13B). In wild type mice fed a high fat diet, the expression of Pitpnm is up-regulated in the liver, as depicted in FIG. 13C. We show in this invention (see FIG. 13D) that the Pitpnm mRNA is expressed during the differentiation into mature adipocyctes. The expression of Pitpnm in metabolic active tissues of wild type mice, as well as the regulation in different animal models used to study metabolic disorders, shows that this gene plays a central role in energy homeostasis.

Taqman analysis revealed that retinal degeneration B2 homolog (Drosophila) (Rdgb2) is expressed in several mammalian tissues, showing highest level of expression in hypothalamus, brain, testis, and lung and higher levels in further tissues, e.g. WAT, BAT, and heart. Furthermore Rdgb2 is expressed on a lower but still robust levels in the muscle, liver, colon, spleen, kidney, pancreas, small intestine, and bone marrow of wild type mice as depicted in FIG. 13E. We found, for example, that the expression of Rdgb2 is down-regulated inWAT, pancreas, and bone marrow of fasted mice compared to wild type mice. We also found that the expression of Rdgb2 is down-regulated inthe pancreas of ob/ob mice compared to wildtype mice (see FIG. 13F). We show in this invention (see FIG. 13G) that the Rdgb2 mRNA is expressed and up-regulated during the differentiation into mature adipocyctes. Therefore, the Rdgb2 protein might play an essential role in adipogenesis. The expression of Rdgb2 in metabolic active tissues of wild type mice, as well as the regulation in different animal models used to study metabolic disorders, shows that this gene plays a central role in energy homeostasis. This finding is supported by the regulation during the differentiation from preadipocytes to mature adipocytes.

Taqman analysis revealed that similar to PYK2 N-terminal domain-interacting receptor 1 (LOC216884) is expressed in several mammalian tissues, showing highest level of expression in hypothalamus and brain and lung Furthermore LOC216884 is expressed on lower but still robust levels in WAT, BAT, muscle, testis, colon, heart, spleen, and kidney of wild type mice as depicted in FIG. 13H. We found, for example, that the expression of LOC216884 is up-regulated in the lung of genetically induced obese mice (ob/ob) compared to wild type mice. (see FIG. 13I). In wild type mice fed a high fat diet, the expression of LOC216884 is up-regulated in the muscle, as depicted in FIG. 13J. The expression of LOC216884 in metabolic active tissues and the regulated expression in tissues of different animal models used to study metabolic disorders shows that this gene plays a central role in energy homeostasis.

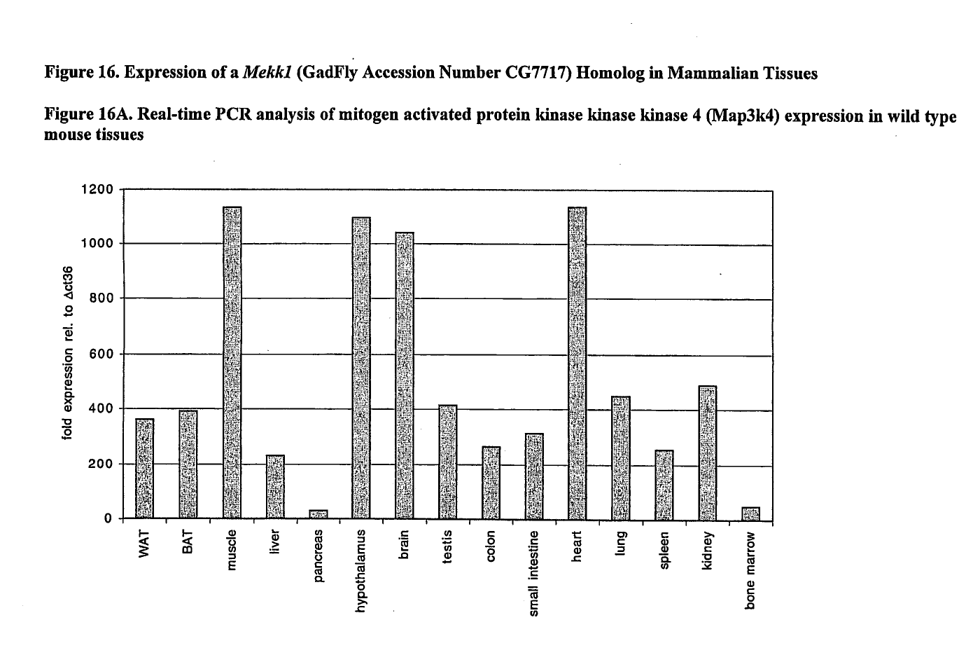

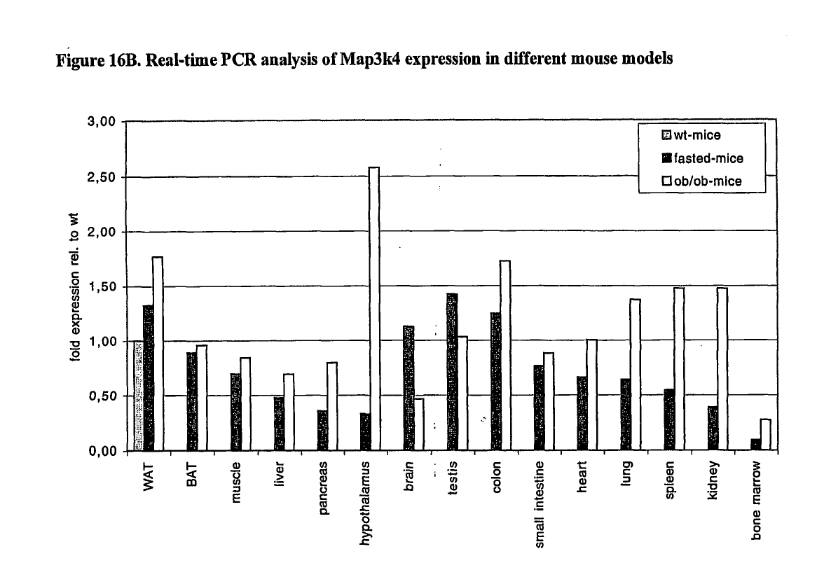

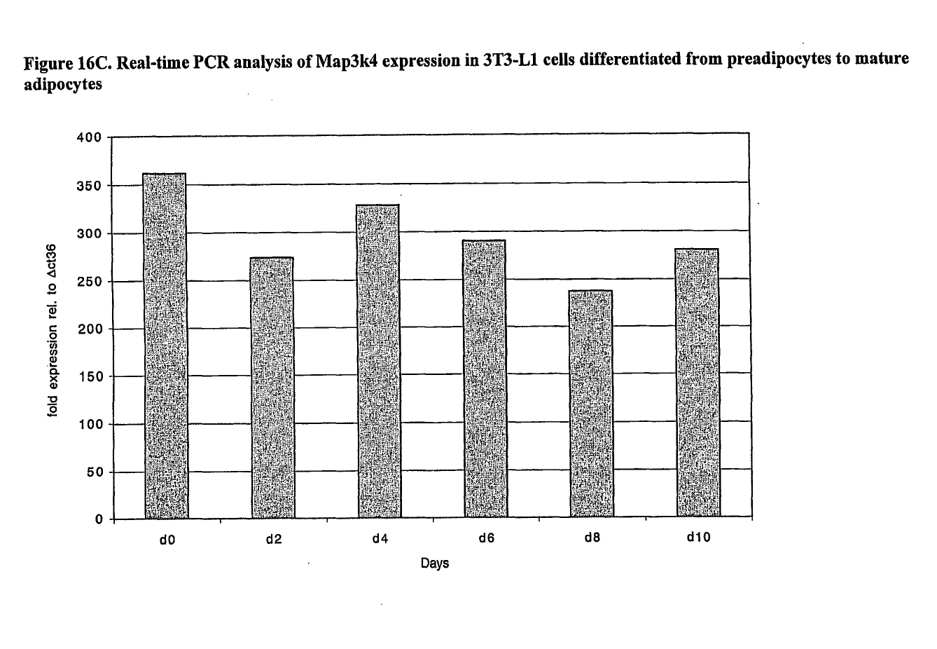

Taqman analysis revealed that mitogen activated protein kinase kinase kinase 4 (Map3k4) is expressed in several mammalian tissues, showing highest level of expression in muscle, hypothalamus, brain, and heart and higher levels in further tissues, e.g. WAT, BAT, liver, testis, colon, small intestine, lung, spleen, and kidney. Furthermore Map3k4 is expressed on a lower but still robust levels in pancreas and bone marrow of wild type mice as depicted in FIG. 16A. We found, for example, that the expression of Map3k4 is down-regulated inpancreas, hypothalamus, kidney, and bone marrow of fasted mice compared to wild type mice. We also found that the expression of Map3k4 is down-regulated inthe brain and bone marrow and up-regulated in the hypothalamus of ob/ob mice compared to wildtype mice (see FIG. 16B). We show in this invention (see FIG. 16C) that the Map3k4 mRNA is expressed during the differentiation into mature adipocyctes. The expression of Map3k4 in metabolic active tissues of wild type mice, as well as the regulation in different animal models used to study metabolic disorders, shows that this gene plays a central role in energy homeostasis.

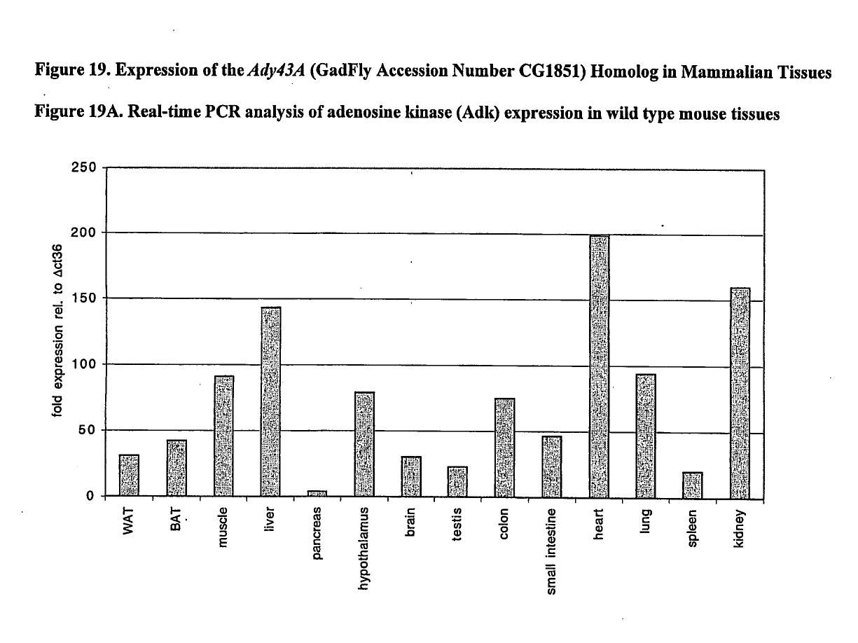

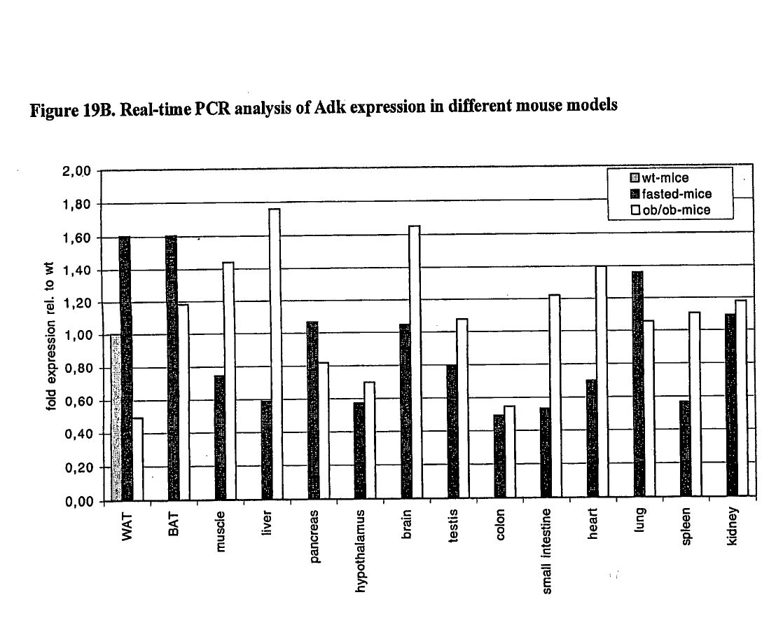

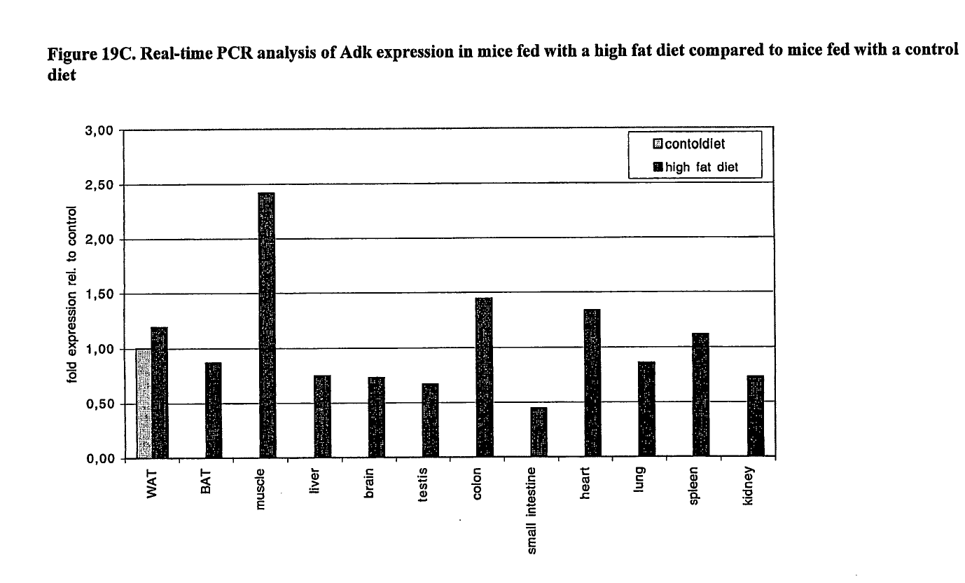

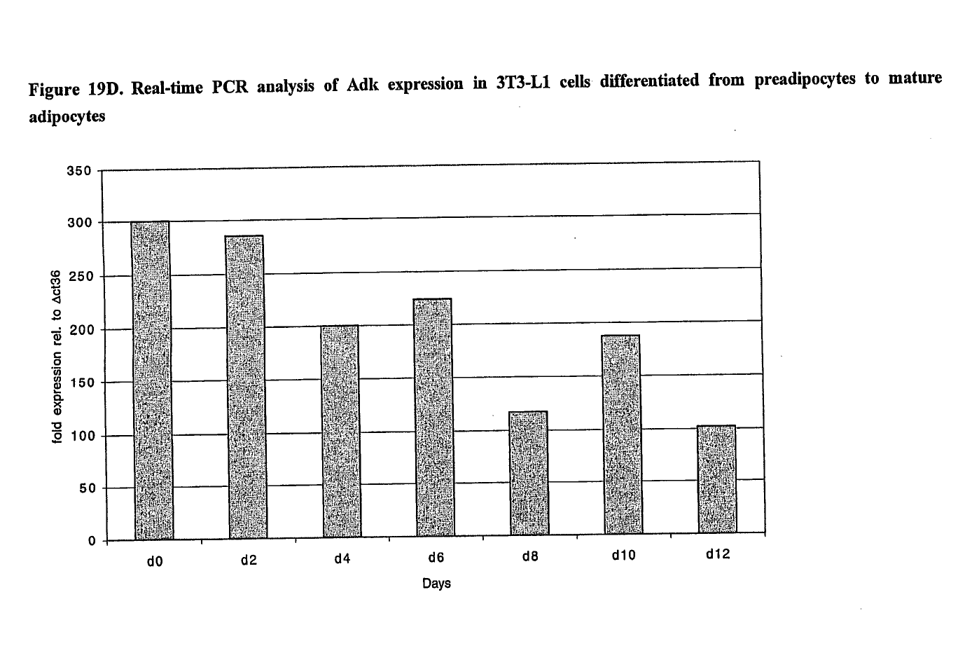

Taqman analysis revealed that adenosine kinase (Adk) is expressed in several mammalian tissues, showing highest level of expression in heart and kidney and higher levels in further tissues, e.g. muscle, liver, hypothalamus, colon, and lung. Furthermore Adk is expressed on a lower but still robust levels in WAT, BAT, brain, testis, small intestine, and spleen of wild type mice as depicted in FIG. 19A. We found, for example, that the expression of Adk is down-regulated inthe colon of fasted mice compared to wild type mice. We also found that the expression of Adk is down-regulated in the WAT of ob/ob mice compared to wildtype mice (see FIG. 19B). In wild type mice fed a high fat diet, the expression of Adk is up-regulated in the muscle and down-regulated inthe small intestine, as depicted in FIG. 19C. We show in this invention (see FIG. 19D) that the Adk mRNA is expressed and down-regulated during the differentiation into mature adipocyctes. Therefore, the Adk protein might play an essential role in adipogenesis. The expression of Adk in metabolic active tissues of wild type mice, as well as the regulation in different animal models used to study metabolic disorders, shows that this gene plays a central role in energy homeostasis. This finding is supported by the down regulation during the differentiation from preadipocytes to mature adipocytes.

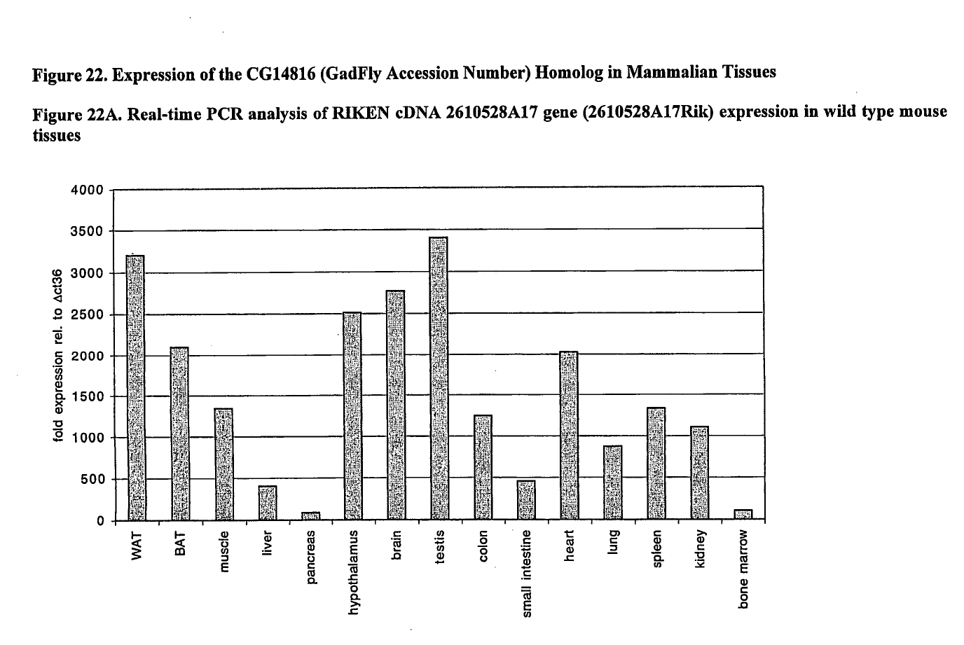

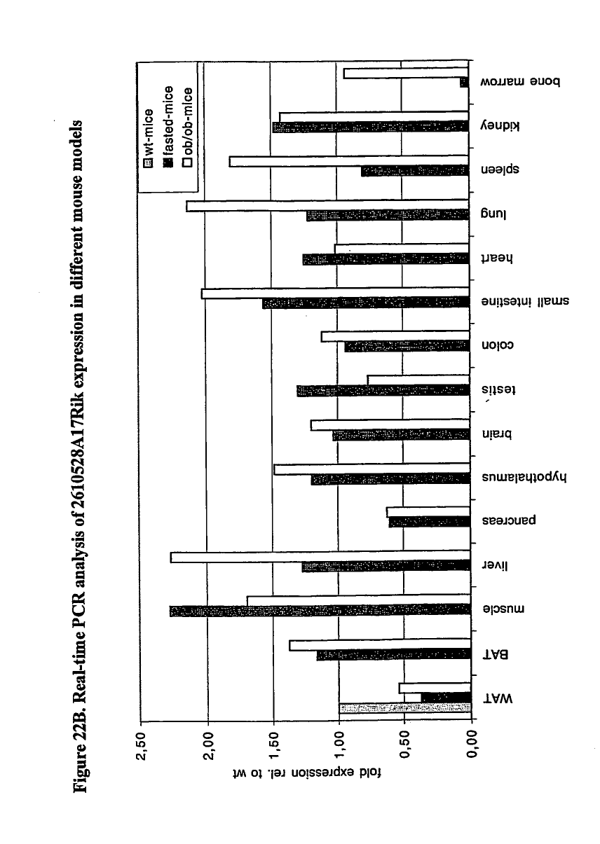

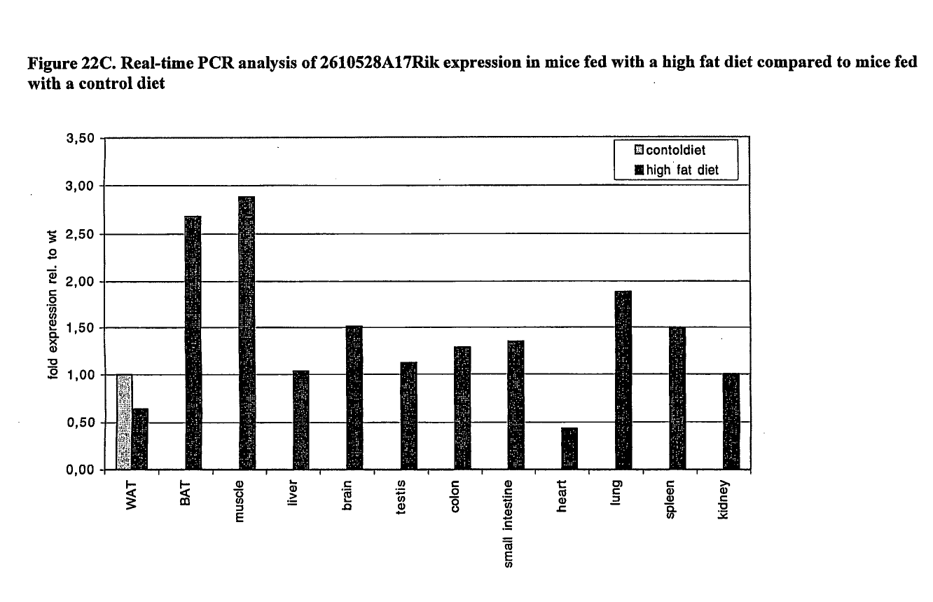

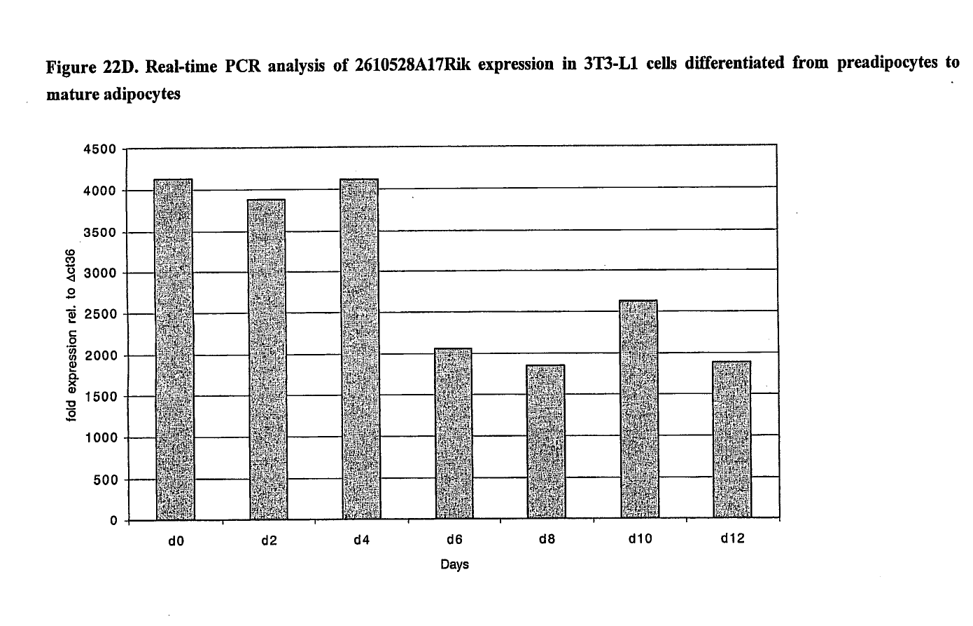

Taqman analysis revealed that RIKEN cDNA 2610528A17 gene (2610528A17Rik) is expressed in several mammalian tissues, showing highest level of expression in WAT and testis and higher levels in further tissues, e.g. BAT, muscle, hypothalamus, brain, colon, heart, lung, spleen, and kidney. Furthermore 2610528A17Rik is expressed on a lower but still robust levels in the liver, small intestine, pancreas, and bone marrow of wild type mice as depicted in FIG. 22A. We found, for example, that the expression of 2610528A17Rik is down-regulated in WAT and bone marrow, and up-regulated in the muscle of fasted mice compared to wild type mice. We also found that the expression of 2610528A17Rik is up-regulated in the liver, small intestine, and lung of ob/ob mice compared to wildtype mice (see FIG. 22B). In wild type mice fed a high fat diet, the expression of 2610528A17Rik is up-regulated in BAT and muscle, and down-regulated in heart, as depicted in FIG. 22C.

We show in this invention (see FIG. 22D) that the 2610528A17Rik mRNA is expressed and down-regulated during the differentiation into mature adipocyctes. Therefore, the 2610528A17Rik protein might play an essential role in adipogenesis. The expression of 2610528A17Rik in metabolic active tissues of wild type mice, as well as the regulation in different animal models used to study metabolic disorders, suggests that this gene plays a central role in energy homeostasis. This finding is supported by the regulation during the differentiation from preadipocytes to mature adipocytes.

### Example 6

### Analysis of the Differential Expression of Transcripts of the Proteins of the Invention in Human Tissues

RNA preparation from human primary adipose tissues was done as described in Example 5. The target preparation, hybridization, and scanning was performed as described in the manufactures manual (see Affymetrix Technical Manual, 2002, obtained from Affmetrix, Santa Clara, USA).

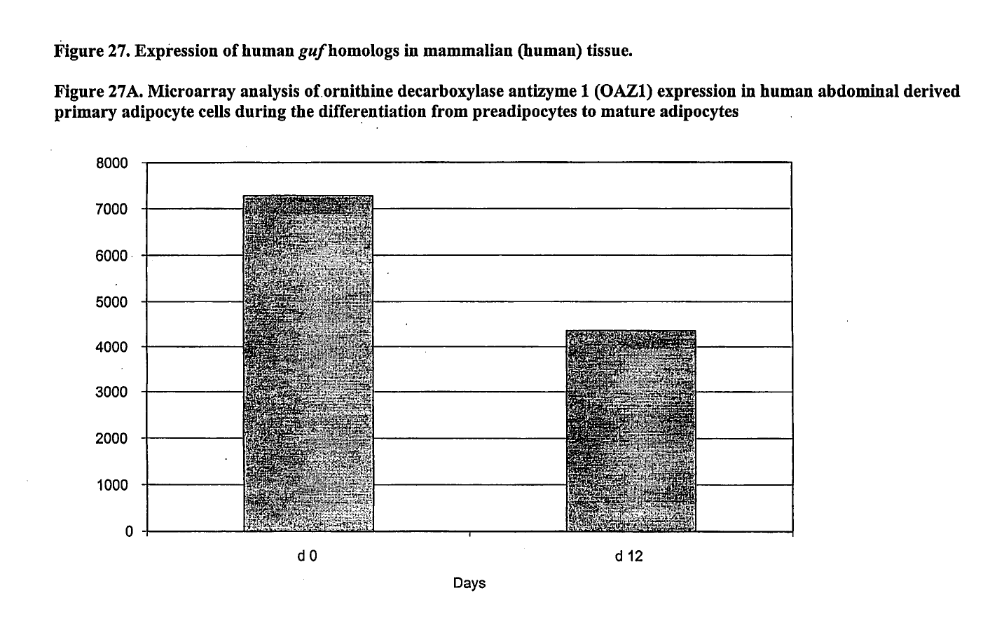

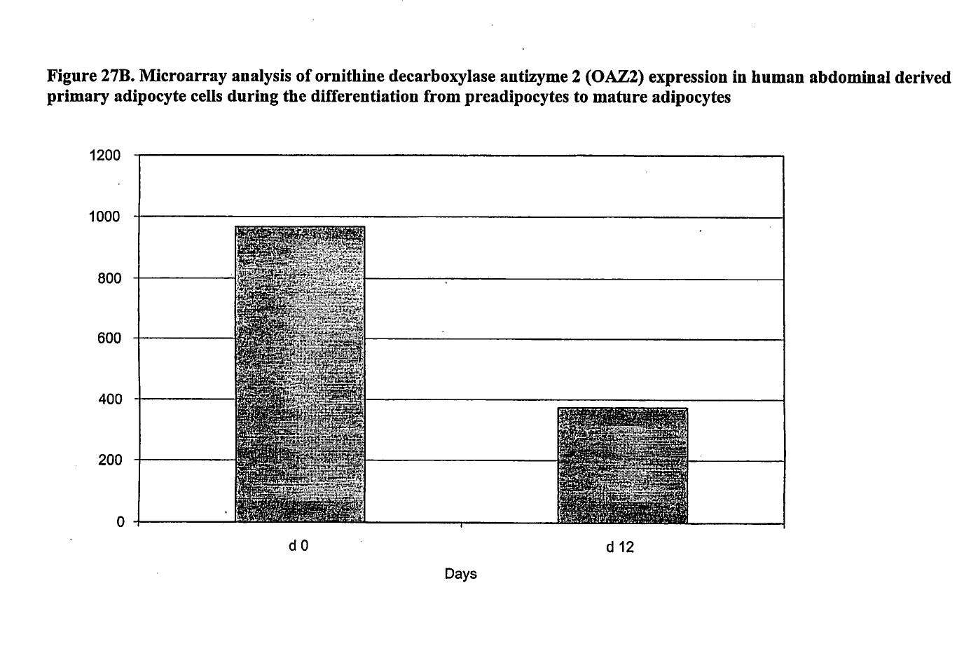

In FIG. 27, the X-axis represents the time axis, shown are day 0 and day 12 of adipocyte differentiation. The Y-axis represents the flourescent intensity. The expression analysis (using Affymetrix GeneChips) of the ornithine decarboxylase antizyme 1 (OAZ1) and ornithine decarboxylase antizyme 2 (OAZ2) genes using human abdominal derived primary adipocytes differentiation, clearly shows differential expression of human OAZ1 and OAZ2 genes in adipocytes. Several independent experiments were done. The experiments further show that the OAZ1 and OAZ2 transcripts are most abundant at day 0 compared to day 12 during differentiation (see FIG. 27).

Thus, the OAZ1 and OAZ2 proteins have to be significantly decreased in order for the preadipocyctes to differentiate into mature adipocycte. Therefore, OAZ1 and OAZ2 in preadipocyctes have the potential to inhibit adipose differentiation. Therefore, OAZ1 and OAZ2 proteins play an essential role in the regulaton of human metabolism, in particular in the regulation of adipogenesis and thus they play an essential role in obesity, diabetes, and/or metabolic syndrome.

For the purpose of the present invention, it will be understood by the person having average skill in the art that any combination of any feature mentioned throughout the specification is explicitly disclosed herewith.

1. A pharmaceutical composition comprising a guf, CG3811, CG30346 (CG8080), rdgB, Mekk1, Ady43A, CG14816, tws, or PP2A-B′ homologous protein or/and a functional fragment thereof, a nucleic acid molecule encoding a guf, CG3811, CG30346 (CG8080), rdgB, Mekk1, Ady43A, CG14816, tws, or PP2A-B′ homologous protein or/and a functional fragment or/and a variant of said nucleic acid molecule or said polypeptide or/and a modulator/effector of said nucleic acid molecule or said protein together with pharmaceutically acceptable carriers, diluents and/or additives.

2. The composition of claim 1, wherein the nucleic acid molecule is a vertebrate or insect guf, CG3811, CG30346 (CG8080), rdgB, Mekk1, Ady43A, CG14816, tws, or PP2A-B′ nucleic acid, particulary encoding a human protein as described in Table 1, or/and a nucleic acid molecule which is complementary thereto or a functional fragment thereof or a variant thereof.

3. The composition of claim 2, wherein said nucleic acid molecule is selected from the group consisting of
(a) a nucleic acid molecule encoding a polypeptide as shown in Table 1, or an isoform, fragment or variant of said polypeptide;
(b) a nucleic acid molecule which comprises or is a nucleic acid molecule as shown in Table 1;
(c) a nucleic acid molecule being degenerated as a result of the genetic code to the nucleic acid sequences as defined in (a) or (b);
(d) a nucleic acid molecule that hybridizes at 50° C. in a solution containing 1 ×SSC and 0.1% SDS to a nucleic acid molecule as defined in claim 2 or as defined in (a) to (c) or/and a nucleic acid molecule which is complementary thereto;
(e) a nucleic acid molecule that encodes a polypeptide which is at least 85%, preferably at least 90%, more preferably at least 95%, more preferably at least 98% and up to 99,6% identical to a human protein as described in Table 1 or as defined in claim 2 or to a polypeptide as described in (a);
(f) a nucleic acid molecule that differs from the nucleic acid molecule of (a) to (e) by mutation and wherein said mutation causes an alteration, deletion, duplication or premature stop in the encoded polypeptide.

4. The composition of claim 1, wherein the nucleic acid molecule is a DNA molecule, particularly a cDNA or a genomic DNA.

5. The composition of claim 1, wherein said nucleic acid encodes a polypeptide contributing to regulating the energy homeostasis or/and the metabolism of triglycerides.

6. The composition of claim 1, wherein said nucleic acid molecule is a recombinant nucleic acid molecule.

7. The composition of claim 1, wherein the nucleic acid molecule is a vector, particularly an expression vector.

8. The composition of claim 1, wherein the polypeptide is a recombinant polypeptide.

9. The composition of claim 8, wherein said recombinant polypeptide is a fusion polypeptide.

10. The composition of claim 1, wherein said nucleic acid molecule is selected from hybridization probes, primers and anti-sense oligonucleotides.

11. The composition of claim 1 which is a diagnostic composition.

12. The composition of claim 1 which is a therapeutic composition.

13. The composition of claim 1 for the manufacture of an agent for detecting or/and verifying, for the treatment, alleviation or/and prevention of metabolic diseases or dysfunctions, including metabolic syndrome, obesity or/and diabetes, as well as related disorders such as eating disorder, cachexia, hypertension, coronary heart disease, hypercholesterolemia, dyslipidemia, osteoarthritis, gallstones, or liver fibrosis, in cells, cell masses, organs and/or subjects.

14. The composition of claim 1 for application in vivo.

15. The composition of claim 1 for application in vitro.

16. Use of a nucleic acid molecule encoding a guf CG3811, CG30346 (CG8080), rdgB, Mekk1, Ady43A, CG14816, tws, or PP2A-B′ homologous protein or a isoform, a functional fragment or a variant thereof, in particular a nucleic acid molecule as described in Table 1, particularly of a nucleic acid molecule according to claim 3 (a), (b), or (c), or/and a polypeptide encoded thereby or/and a functional fragment or/and a variant of said nucleic acid molecule or said polypeptide or/and a modulator/effector of said nucleic acid molecule or polypeptide for the manufacture of a medicament for the treatment of obesity, diabetes, or/and metabolic syndrome for controlling the function of a gene or/and a gene product which is influenced or/and modified by a guf, CG3811, CG30346 (CG8080), rdgB, Mekk1, Ady43A, CG14816, tws, or PP2A-B′ homologous polypeptide, particularly by a polypeptide according to claim 3.

17. Use of a nucleic acid molecule encoding a guf, CG3811, CG30346 (CG8080), rdgB, Mekk1,Ady43A, CG14816, tws, or PP2A-B′ homologous protein or an isoform, a functional fragment or variant thereof, in particular a nucleic acid molecule as described in Table 1, particularly of a nucleic acid molecule according to claim 3 (a), (b), or (c), or/and a polypeptide encoded thereby or/and a functional fragment or/and a variant of said nucleic acid molecule or said polypeptide or/and a modulator/effector of said nucleic acid molecule or said polypeptide for identifying substances capable of interacting with a guf, CG3811, CG30346 (CG8080), rdgB, Mekk1, Ady43A, CG14816, tws, or PP2A-B′ homologous polypeptide, particularly with a polypeptide according to claim 3.

18. A non-human transgenic animal exhibiting a modified expression of a guf, CG3811, CG30346 (CG8080), rdgB, Mekk1, Ady43A, CG14816, tws, or PP2A-B′ homologous polypeptide, particularly of a polypeptide according to claim 3.

19. The animal of claim 18, wherein the expression of the guf, CG3811, CG30346 (CG8080), rdgB, Mekk1, Ady43A, CG14816, tws, or PP2A-B′ homologous polypeptide, is increased and/or reduced.

20. A recombinant host cell exhibiting a modified expression of a guf, CG3811, CG30346 (CG8080), rdgB, Mekk1, Ady43A, CG14816, tws, or PP2A-B′ homologous polypeptide, particularly of a polypeptide according to claim 3.

21. The cell of claim 20 which is a human cell.

22. A method of identifying a (poly)peptide involved in the regulation of energy homeostasis or/and metabolism of triglycerides in a mammal comprising the steps of
(a) contacting a collection of (poly)peptides with a guf, CG3811, CG30346 (CG8080), rdgB, Mekk1, Ady43A, CG14816, tws, or PP2A-B′ homologous polypeptide, particularly with a polypeptide according to claim 3, or a functional fragment thereof under conditions that allow binding of said (poly)peptides;
(b) removing (poly)peptides which do not bind and
(c) identifying (poly)peptides that bind to said guf, CG3811, CG30346 (CG8080), rdgB, Mekk1, Ady43A, CG14816, tws, or PP2A-B′ homologous polypeptide.

23. A method of screening for an agent which modulates/effects the interaction of a guf, CG3811, CG30346 (CG8080), rdgB, Mekk1, Ady43A, CG14816, tws, or PP2A-B′ homologous polypeptide, particularly of a polypeptide according to claim 3, with a binding target, comprising the steps of
(a) incubating a mixture comprising
(aa) a guf, CG3811, CG30346 (CG8080), rdgB, Mekk1, Ady43A, CG14816, tws, or PP2A-B′ homologous polypeptide, particularly a polypeptide according to claim 3, or a functional fragment thereof;
(ab) a binding target/agent of said polypeptide or functional fragment thereof; and
(ac) a candidate agent under conditions whereby said polypeptide or functional fragment thereof specifically binds to said binding target/agent at a reference affinity;
(b) detecting the binding affinity of said polypeptide or functional fragment thereof to said binding target to determine an affinity for the agent; and
(c) determining a difference between affinity for the agent and the reference affinity.

24. A method for screening for an agent, which modulates/effects the activity of a guf, CG3811, CG30346 (CG8080), rdgB, Mekk1, Ady43A, CG14816, tws, or PP2A-B′ homologous polypeptide, particularly of a polypeptide according to claim 3, comprising the steps of
(a) incubating a mixture comprising
(aa) said polypeptide or a functional fragment thereof;
(ab) a candidate agent under conditions whereby said polypeptide or functional fragment thereof has a reference activity;
(b) detecting the activity of said polypeptide or functional fragment thereof to determine an activity in presence of the agent; and
(c) determining a difference between the activity in the presence of the agent and the reference activity.

25. A method of producing a composition comprising mixing the (poly)peptide identified by the method of claim 22 with a pharmaceutically acceptable carrier, diluent and/or additive.

26. The method of claim 25 wherein said composition is a pharmaceutical composition for preventing, alleviating or treating of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.

27. Use of a (poly)peptide as identified by the method of claim 22 for the preparation of a pharmaceutical composition for the treatment, alleviation and/or prevention of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosiss.

28. Use of a nucleic acid molecule as defined in claim 1 for the preparation of a medicament for the treatment, alleviation or/and prevention of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.

29. Use of a polypeptide as defined in claim 1 for the preparation of a medicament for the treatment, alleviation or/and prevention of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.

30. Use of a vector as defined in claim 7 for the preparation of a medicament for the treatment, alleviation or/and prevention of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.

31. Use of a host cell as defined in claim 20 for the preparation of a medicament for the treatment, alleviation or/and prevention of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosis.

32. Use of a guf, CG3811, CG30346 (CG8080), rdgB, Mekk1, Ady43A, CG14816, tws, or PP2A-B′ homologous nucleic acid molecule or of a functional fragment thereof for the production of a non-human transgenic animal which over- or under-expresses the guf, CG3811, CG30346 (CG8080), rdgB, Mekk1, Ady43A, CG14816, tws, or PP2A-B′ homologous gene product.

33. Kit comprising at least one of
(a) a guf, CG3811, CG30346 (CG8080), rdgB, Mekk1, Ady43A, CG14816, tws, or PP2A-B′ homologous nucleic acid molecule or a functional fragment thereof;
(b) a guf, CG3811, CG30346 (CG8080), rdgB, Mekk1, Ady43A, CG14816, tws, or PP2A-B′ homologous amino acid molecule or a functional fragment or an isoform thereof;
(c) a vector comprising the nucleic acid of (a);
(d) a host cell comprising the nucleic acid of (a) or the vector of (c);
(e) a polypeptide encoded by the nucleic acid of (a);
(f) a fusion polypeptide encoded by the nucleic acid of (a);
(g) an antibody, an aptamer or/and another modulator/effector of the nucleic acid of (a) or/and the polypeptide of (b), (e), or/and (f) and
(h) an anti-sense oligonucleotide of the nucleic acid of (a).

34. A method of producing a composition comprising mixing the agent identified by the method of claim 23 with a pharmaceutically acceptable carrier, diluent and/or additive.

35. Use of an agent as identified by the method of claim 23 for the preparation of a pharmaceutical composition for the treatment, alleviation and/or prevention of metabolic diseases or dysfunctions, including obesity, diabetes, or/and metabolic syndrome, as well as related disorders such as eating disorder, cachexia, hypertension, coronary heart disease, hypercholesterolemia, dyslipidemia, osteoarthritis, gallstones, or liver fibrosiss.


SEQUENCE LISTING (37 sequences)

SEQ ID NO: 1 - DNA, 22 bp, Mus musculus
cagcagcgagagttctagggtt

SEQ ID NO: 2 - DNA, 17 bp, Mus musculus
aggagcaccaccgaggc

SEQ ID NO: 3 - DNA, 30 bp, Mus musculus
cccttaattgctgtagtaacctgggtccgg

SEQ ID NO: 4 - DNA, 24 bp, Mus musculus
tattttgccgttgagtaagtgtcc

SEQ ID NO: 5 - DNA, 18 bp, Mus musculus
caccacagaggccctgga

SEQ ID NO: 6 - DNA, 24 bp, Mus musculus
agctccagtgctgcaggcacattg

SEQ ID NO: 7 - DNA, 21 bp, Mus musculus
caaatcgatcggaaggacaga

SEQ ID NO: 8 - DNA, 18 bp, Mus musculus
ccacctcgaagcccatgt

SEQ ID NO: 9 - DNA, 22 bp, Mus musculus
tgccctgctgcgagcctttagc

SEQ ID NO: 10 - DNA, 15 bp, Mus musculus
gccttgcggtgggca

SEQ ID NO: 11 - DNA, 19 bp, Mus musculus
agcgcaactgtccgaacaa

SEQ ID NO: 12 - DNA, 24 bp, Mus musculus
acacccctggagccacaaagtggg

SEQ ID NO: 13 - DNA, 18 bp, Mus musculus
tgcctgtgcgatacacgc

SEQ ID NO: 14 - DNA, 21 bp, Mus musculus
aaccacctgaactcaccacga

SEQ ID NO: 15 - DNA, 26 bp, Mus musculus
ttccttccctgaagccttacggaggt

SEQ ID NO: 16 - DNA, 20 bp, Mus musculus
cacacaggtcaagatccgga

SEQ ID NO: 17 - DNA, 17 bp, Mus musculus
agcgcccattcagcacc

SEQ ID NO: 18 - DNA, 25 bp, Mus musculus
cacttccaaccaccgagcgagtgac

SEQ ID NO: 19 - DNA, 18 bp, Mus musculus
tgagccgcagcaacattg

SEQ ID NO: 20 - DNA, 20 bp, Mus musculus
ctggataggaaggcctggtg

SEQ ID NO: 21 - DNA, 24 bp, Mus musculus
ccgaagcaacggcactgaggactc

SEQ ID NO: 22 - DNA, 19 bp, Mus musculus
aagtggctgcgtaagcgaa

SEQ ID NO: 23 - DNA, 19 bp, Mus musculus
acatcattggcacggtggt

SEQ ID NO: 24 - DNA, 27 bp, Mus musculus
ccaggtcaagttgcggaacgttactgc

SEQ ID NO: 25 - DNA, 17 bp, Mus musculus
cagcccatcatcgccaa

SEQ ID NO: 26 - DNA, 20 bp, Mus musculus
gcatcgctgattctgttgca

SEQ ID NO: 27 - DNA, 26 bp, Mus musculus
ttgcagcagctcaagaacgatgcact

SEQ ID NO: 28 - DNA, 20 bp, Mus musculus
agcccaaaaagctcaaggtg

SEQ ID NO: 29 - DNA, 21 bp, Mus musculus
ttccccattccaaatagcaca

SEQ ID NO: 30 - DNA, 22 bp, Mus musculus
agcgccacaagcgctgagtgaa

SEQ ID NO: 31 - DNA, 19 bp, Mus musculus
tgtccctcaacaacggcag

SEQ ID NO: 32 - DNA, 19 bp, Mus musculus
tcctgagtgccacacgacc

SEQ ID NO: 33 - DNA, 23 bp, Mus musculus
cacccacctggtgattcgaccca

SEQ ID NO: 34 - protein, 1349 aa, Homo sapiens
MIIKEYRIPLPMTVEEYRIAQLYMIQKKSRNETYGEGSGVEILENRPYTDGPGGSGQYTHKVYHVGMHIPSWFRSILPKAALRVVEESWNAYPYTRTRFTCPFVEKFSIDIETFYKTDAGENPDVFNLSPVEKNQLTIDFIDIVKDPVPHNEYKTEEDPKLFQSTKTQRGPLSENWIEEYKKQVFPIMCAYKLCKVEFRYWGMQSKIERFIHDTGLRRVMVRAHRQAWCWQDEWYGLSMENIRELEKEAQLMLSRKMAQFNEDGEEATELVKHEAVSDQTSGEPPEPSSSNGEPLVGRGLKKQWSTSSKSSRSSKRGASPSRHSISEWRMQSIARDSDESSDDEFFDAHEDLSDTEEMFPKDITKWSSNDLMDKIESPEPEDTQDGLYRQGAPEFRVASSVEQLNIIEDEVSQPLAAPPSKIHVLLLVLHGGTILDTGAGDPSSKKGDANTIANVFDTVMRVHYPSALGRLAIRLVPCPPVCSDAFALVSNLSPYSHDEGCLSSSQDHIPLAALPLLATSSPQYQEAVATVIQRANLAYGDFIKSQEGMTFNGQVCLIGDCVGGILAFDALCYSNQPVSESQSSSRRGSVVSMQDNDLLSPGILMNAAHCCGGGGGGGGGGGSSGGGGSSGGSSLESSRHLSRSNVDIPRSNGTEDPKRQLPRKRSDSSTYELDTIQQHQAFLSSLHASVLRTEPCSRHSSSSTMLDGTGALGRFDFEITDLFLFGCPLGLVLALRKTVIPALDVFQLRPACQQVYNLFHPADPSASRLEPLLERRFHALPPFSVPRYQRYPLGDGCSTLLADVLQTHNAAFQEHGAPSSPGTAPASRGFRRASEISIASQVSGMAESYTASSIAQKAPDALSHTPSVRRLSLLALPAPSPTTPGPHPPARKASPGLERAPGLPELDIGEVAAKWWGQKRIDYALYCPDALTAFPTVALPHLFHASYWESTDVVSFLLRQVMRHDNSSILELDGKEVSVFTPSKPREKWQRKRTHVKLRNVTANHRINDALANEDGPQVLTGRFMYGPLDMVTLTGEKVDVHIMTQPPSGEWLYLDTLVTNNSGRVSYTIPESHRLGVGVYPIKMVVRGDHTFADSYITVLPKGTEFVVFSIDGSFAASVSIMGSDPKVRAGAVDVVRHWQDLGYLIIYVTGRPDMQKQRVVAWLAQHNFPHGVVSFCDGLVHDPLRHKANFLKLLISELHLRVHAAYGSTKDVAVYSAISLSPMQIYIVGRPTKKLQQQCQFITDGYAAHLAQLKYSHRARPARNTATRMALRKGSFGLPGQGDFLRSRNHLLRTISAQPSGPSHRHERTQSQADGEQRGQRSMSVAAGCWGRAMTGRLEPGAAAGPK

SEQ ID NO: 35 - protein, 974 aa, Homo sapiens
MAKAGRAGGPPPGGGAPWHLRNVLSDSVESSDDEFFDAREEMAEGKNAILIGMSQWNSNDLVEQIETMGKLDEHQGEGTAPCTSSILQEKQRELYRVSLRRQRFPAQGSIEIHEDSEEGCPQRSCKTHVLLLVLHGGNILDTGAGDPSCKAADIHTFSSVLEKVTRAHFPAALGHILIKFVPCPAICSEAFSLVSHLNPYSHDEGCLSSSQDHVPLAALPLLAISSPQYQDAVATVIERANQVYREFLKSSDGIGFSGQVCLIGDCVGGLLAFDAICYSAGPSGDSPASSSRKGSISSTQDTPVAVEEDCSLASSKRLSKSNIDISSGLEDEEPKRPLPRKQSDSSTYDCEAITQHHAFLSSIHSSVLKDESETPAAGGPQLPEVSLGRFDFDVSDFFLFGSPLGLVLAMRRTVLPGLDGFQVRPACSQVYSFFHCADPSASRLEPLLEPKFHLVPPVSVPRYQRFPLGDGQSLLLADALHTHSPLFLEGSSRDSPPLLDAPASPPQASRFQRPGRRMSEGSSHSESSESSDSMAPVGASRITAKWWGSKRIDYALYCPDVLTAFPTVALPHLFHASYWESTDVVAFILRQVMRYESVNIKESARLDPAALSPANPREKWLRKRTQVKLRNVTANHRANDVIAAEDGPQVLVGRFMYGPLDMVALTGEKVDILVMAEPSSGRWVHLDTEITNSSGRITYNVPRPRRLGVGVYPVKMVVRGDQTCAMSYLTVLPRGMECVVFSIDGSFAASVSIMGSDPKVRPGAVDVVRHWQDLGYMILYITGRPDMQKQRVVSWLSQHNFPQGMIFFSDGLVHDPLRQKAIFLRNLMQECFIKISAAYGSTKDISVYSVLGLPASQIFIVGRPTKKYQTQCQFLSEGYAAHLAVLEASHRSRPKKNNSRMILRKGSFGLHAQPEFLRKRNHLRRTMSVQQPDPPAANPKPERAQSQPESDKDHERPLPALSWARGPPKFESVP

SEQ ID NO: 36 - protein, 1244 aa, Homo sapiens
MLIKEYHILLPMSLDEYQVAQLYMIQKKSREESSGEGSGVEILANRPYTDGPGGSGQYTHKVYHVGSHIPGWFRALLPKAALQVEEESWNAYPYTRTRYTCPFVEKFSIEIETYYLPDGGQQPNVFNLSGAERRQRILDTIDIVRDAVAPGEYKAEEDPRLYHSVKTGRGPLSDDWARTAAQTGPLMCAYKLCKVEFRYWGMQAKIEQFIHDVGLRRVMLRAHRQAWCWQDEWTELSMADIRALEEETARMLAQRMAKCNTGSEGSEAQPPGKPSTEARSAASNTGTPDGPEAPPGPDASPDASFGKQWSSSSRSSYSSQHGGAVSPQSLSEWRMQNIARDSENSSEEEFFDAHEGFSDSEEVFPKEMTKWNSNDFIDAFASPVEAEGTPEPGAEAAKGIEDGAQAPRDSEGLDGAGELGAEACAVHALFLILHSGNILDSGPGDANSKQADVQTLSSAFEAVTRIHFPEALGHVALRLVPCPPICAAAYALVSNLSPYSHDGDSLSRSQDHIPLAALPLLATSSSRYQGAVATVIARTNQAYSAFLRSPEGAGFCGQVALIGDGVGGILGFDALCHSANAGTGSRGSSRRGSMNNELLSPEFGPVRDPLADGVEGLGRGSPEPSALPPQRIPSDMASPEPEGSQNSLQAAPATTSSWEPRRASTAFCPPAASSEAPDGPSSTARLDFKVSGFFLFGSPLGLVLALRKTVMPALEAAQMRPACEQIYNLFHAADPCASRLEPLLAPKFQAIAPLTVPRYQKFPLGDGSSLLLADTLQTHSSLFLEELEMLVPSTPTSTSGAFWKGSELATDPPAQPAAPSTTSEVVKILERWWGTKRIDYSLYCPEALTAFPTVTLPHLFHASYWESADVVAFILRQVIEKERPQLAECEEPSIYSPAFPREKWQRKRTQVKIRNVTSNHRASDTVVCEGRPQVLSGRFMYGPLDVVTLTGEKVDVYIMTQPLSGKWIHFGTEVTNSSGRLTFPVPPERALGIGVYPVRMVVRGDHTYAECCLTVVARGTEAVVFSIDGSFTASVSIMGSDPKVRAGAVDVVRHWQDSGYLIVYVTGRPDMQKHRVVAWLSQHNFPHGVVSFCDGLTHDPLRQKAMFLQSLVQEVGLNIVAGYGSPKDVAVYTALGLSPSQTYIVGRAVRKLQAQCQFLSDGYVAHLGQLEAGSHSHASSGPPRAALGKSSYGVAAPVDFLRKQSQLLRSRGPSQAEREGPGTPPTTLARGKARSISLKLDSEE

SEQ ID NO: 37 - protein, 1250 aa, Drosophila melanogaster
MLIKEYRIPLPLTVEEYRIAQLYMIAKKSREESHGEGSGVEIIINEPYKDGPGGNGQYTKKIYHVGNHLPGWIKSLLPKSALTVEEEAWNAYPYTRTRYTCPFVEKFSLDIETYYYPDNGYQDNVFQLSGSDLRNRIVDVIDIVKDQLWGGDYVKEEDPKHFVSDKTGRGPLAEDWLEEYWREVKGKKQPTPRNMSLMTAYKICRVEFRYWGMQTKLEKFIHDVALRKMMLRAHRQAWAWQDEWFGLTIEDIRELERQTQLALAKKMGGGEECSDDSVSEPYVSTAATAASTTGSERKKSAPAVPPIVTQQPPSAEASSDEEGEEEEDDDEDENDAIGTGVDLSANQGGSAQRSRSQSIQMAQKGKFGSKGALHSPVGSAHSFDLQVANWRMERLEVDSKSNSDEEFFDCLDTNETNSLAKWSSLELLGEGDDSPPPHGGPSSAASVGGRGNSRQEDSIFNQDFLMRVASERGNKRQLRSSASVDRSHDSSPPGSPSTPSCPTTILILVVHAGSVLDAASELTAKKSDVTTFRGSFEAVMRQHYPSLLTHVTIKMVPCPSICTDALGILSSLSPYSFDASPSAADIPNIADVPIGAIPLLSVASPEFHETVNKTVAAANIVYHEFLKSEEGHGFSGQIVMLGDSMGSLLAYEALCRSNGSQPGTASGASNSGGDAATNINTHNPLSPRNSRLDDDERFIEADLDAKRLLVAPSPRRRRSSSSSDSRATKLDFEVCDFFMFGSPLSVVLAARKLHDAKAALPRPNCHQVYNLFHPTDPIASRLEPLLSARFSILAPVNVPRYAKYPLGNGQPLHLLEVIQSHPQHFNDGNNLLAGRRLSDASMQSTISGLIENVSLSTIHALQNKWWGTKRLDYALYCPEGLSNFPAHALPHLFHASYWESPDVIAFILRQIGKFEGIPFVGSNDDKDNASFHPGQPREKWIKKRTSVKLKNVAANHRANDVIVQEGREQRLNARFMYGPLDMITLHGEKVDVHIMKDPPAGEWTFLSTEVTDKNGRISYSIPDQVSLGYGIYPVKMVVRGDHTSVDCYMAVVPPLTECVVFSIDGSFTASMSVTGRDPKVRAGAVDVCRHWQELGYLLIYITGRPDMQQQRVVSWLSQHNFPHGLISFADGLSTDPLGHKTAYLNNLVQNHGISITAAYGSSKDISVYTNVGMRTDQIFIVGKVGKKLQSNATVLSDGYAAHLAGLQAVGGSRPAKGNARMVIPRGCFNLPGQTANPRRRRLHEQATNEN